(19) United States
(12) Patent Application Publication
(10) Pub. No.: US 2005/0267148 A1
(43) Pub. Date: Dec. 1, 2005

(54) Benzimidazole derivative

(75) Inventors: Naoki Tsuchiya, Tokyo (JP); Tsuyoshi Mizuno, Tokyo (JP); Hiroshi Saitou, Tokyo (JP); Yoshiyuki Matsumoto, Tokyo (JP); Susumu Takeuchi, Tokyo (JP); Naoki Hase, Tokyo (JP)

Correspondence Address: SUGHRUE MION, PLLC, 2100 PENNSYLVANIA AVENUE, N.W., SUITE 800, WASHINGTON, DC, 20037, US

(73) Assignee: TEIJIN LIMITED

(21) Appl. No.: 11/129,508

(22) Filed: May 16, 2005

Related U.S. Application Data
(63) Continuation-in-part of application No. 10/777,067, filed on Feb. 13, 2004.
Application No. 10/777,067 is a continuation of application No. 10/169,866, filed on Jul. 10, 2002, now abandoned.
(63) Continuation-in-part of application No. 10/963,710, filed on Oct. 14, 2004.
Application No. 10/963,710 is a continuation of application No. 09/743,483, filed on Jan. 10, 2001, now abandoned.

(30) Foreign Application Priority Data
Jul. 15, 1998 (JP) 10-200250(PAT.)
Jan. 17, 2000 (JP) 2000-007533(PAT.)
Dec. 25, 2000 (JP) 2000-392303(PAT.)

(51) Int. Cl. C07D 471/02; A61K 31/4745; A61K 31/4184

(52) U.S. Cl. 514/303; 514/394; 546/118; 548/308.4

(57) ABSTRACT

The present invention is a thiobenzimidazole derivative represented by the following formula (1) 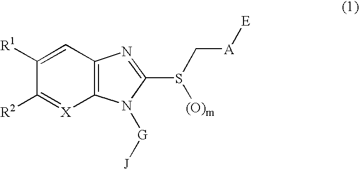 or a medically acceptable salt thereof wherein said thiobenzimidazole derivative and a medically acceptable salt thereof have a potent activity of inhibiting human chymase. Thus, they are potential preventive and/or therapeutic agents clinically applicable to various diseases in which human chymase is involved.

The present application is a continuation-in-part of U.S. application Ser. No. 10/777,067 filed Feb. 13, 2004, which is a Continuation Application of U.S. application Ser. No. 10/169,866, fild Jul. 10, 2002 (now abandoned) which is a National Stage application filed under §371 of PCT/JP01/00271 filed on Jan. 17, 2001; and of U.S. application Ser. No. 10/963,710 filed Oct. 14, 2004, which is a Continuation Application of U.S. application Ser. No. 09/743,483, filed Jan. 10, 2001 (now abandoned), which is a National Stage Application filed under §371 of PCT Application No. PCT/JP99/0379, filed Jul. 14, 1999; the entire disclosures of which are incorporated herein by reference.

### BACKGROUND OF THE INVENTION

1. Technical Field

The present invention relates to thiobenzimidazole derivatives represented by the formula (1) and, more specifically, thiobenzimidazole derivatives useful as inhibitors of human chymase activity.

2. Background Art

Chymase is one of the neutral proteases present in mast cell granules, and is deeply involved in a variety of biological processes in which mast cells participate. Various effects have been reported including, for example, the promotion of degranulation from mast cells, the activation of interleukin-1β (IL-1β), the activation of matrix protease, the decomposition of fibronectin and type IV collagen, the promotion of the release of transforming growth factor-β (TGF-β), the activation of substance P and vasoactive intestinal polypeptide (VIP), the conversion of angiotensin I (Ang I) to Ang II, the conversion of endothelin, and the like.

The above indicates that inhibitors of said chymase activity may be promising as preventive and/or therapeutic agents for diseases of respiratory organs such as bronchial asthma, inflammatory/allergic diseases, for example allergic rhinitis, atopic dermatitis, and urticaria; diseases of circulatory organs, for example sclerosing vascular lesions, intravascular stenosis, disturbances of peripheral circulation, renal failure, and cardiac failure; diseases of bone/cartilage metabolism such as rheumatoid arthritis and osteoarthritis, and the like.

As inhibitors of chymase activity, there are known triazine derivatives (Japanese Unexamined Patent Publication (Kokai) No. 8-208654); hydantoin derivatives (Japanese Unexamined Patent Publication (Kokai) No. 9-31061); imidazolidine derivatives (PCT Application WO 96/04248); quinazoline derivatives (PCT Application WO 97/11941); heterocyclic amide derivatives (PCT Application WO 96/33974); and the like. However, the structures of these compounds are entirely different from those of the compounds of the present invention.

On the other hand, an art related to the compounds of the present invention is disclosed in U.S. Pat. No. 5,124,336. Said specification describes thiobenzimidazole derivatives as having an activity of antagonizing thromboxane receptor. The specification, however, makes no mention of the activity of said compounds to inhibit human chymase.

Thus, it is an object of the present invention to provide novel compounds that are potential and clinically applicable inhibitors of human chymase.

### SUMMARY OF THE INVENTION

Thus, after intensive research to attain the above objective, the applicants of the present invention have found the following 1 to 21 and have thereby completed the present invention.

1. A thiobenzimidazole compound or medically acceptable salt thereof represented by the following formula (1): 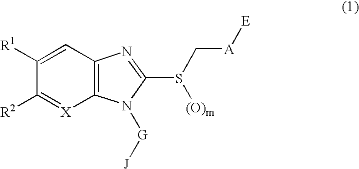
wherein,
R¹ and R², simultaneously or respectively independently represent a hydrogen atom, fluorine atom, chlorine atom, bromine atom, iodine atom, trifluoromethyl group, cyano group, hydroxyl group, methyl group, ethyl group, (n- or i-)propyl group, (n-, i-, s- or t-)butyl group, methoxy group, ethoxy group, (n- or i-)propyloxy group, (n-, i-, s- or t-)abutyloxy group, or R¹ and R² together represent —O—CH₂—O—, —O—CH₂—CH₂—O— or —CH₂—CH₂—CH₂— in this case, the carbon atoms may be substituted with one or a plurality of methyl groups, ethyl groups, (n- or i-)propyl groups or (n-, i-, s- or t-)butyl groups;
A represents a substituted or non-unsubstituted, methylene group, ethylene group, (n- or i-)propylene group or (n-, i- or t-)butylenes group, substituted or non-substituted phenylene group, indenylene group or naphthylene group, substituted or non-substituted pyridylene group, furanylene group, thiophenylene group, pyrimidylene group, benzophenylene group, benzimidazolene group, quinolylene group, indolene group or benzoathiazolene group and substitution groups here are represented by a halogen atom, OH, NO₂, CN, methyl group, ethyl group, (n- or i-)propyl group, (n-, i-, s- or t-)butyl group, methoxy group, ethoxy group, (n- or i-)propyloxy group, (n-, i-, s- or t-)butyloxy group, in this case, substitution groups may be acetal-bonded at mutually adjacent sites, methylthio group, ethylthio group, (n- or i-)propylthio group, (n-, i-, s- or t-)butylthio, methylsulfonyl group, ethylsulfonyl group, (n- or i-)propylsulfonyl group, (n-, i-, s- or t-)butylsulfonyl group, acetyl group, ethylcarbonyl group, (n- or i-)propylcarbonyl group, acetylamino group, ethylcarbonylamino group, (n- or i-)propylcarbonylamino group, (n-, i-, s- or t-)butylcarbonylamino group, trifluoromethyl group or trifluoromethoxy group, and one or a plurality of these may be respectively and independently substituted at an arbitrary location of a ring or alkyl group;
E represents COOR³, SO₃R³, CONHR³, SO₂NHR³, a tetrazole group, 5-oxo-1,2,4-oxadiazole group or 5-oxo-1,2,4-thiadiazole group, wherein R³ represents a hydrogen atom, methyl group, ethyl group, (n- or i-)propyl group or (n-, i-, s- or t-)butyl group;
G represents a substituted or non-substituted methylene group, ethylene group, (n- or i-)propylene group or (n-, i- or t-)butylene group, and one or a plurality of O, S, SO₂ or NR³ may be intermediately contained therein, wherein R³ is the same as previously defined, and substitution groups here are represented by a halogen atom, OH, NO₂, CN, methyl group, ethyl group, (n- or i-)propyl group, (n-, i-, s- or t-)butyl group, methoxy group, ethoxy group, (n- or i-)propyloxy group, (n-, i-, s- or t-)butyloxy group, trifluoromethyl group, trifluoromethoxy group or oxo group;
m represents an integer of 0-2;
when m is 0 and A is a substituted or non-substituted methylene group, ethylene group, (n- or i-)propylene group or (n-, i- or t-)butylene group, J represents a substituted or non-substituted (n- or i-)propyl group, (n-, i-, s- or t-)butyl group, (n-, i-, ne- or t-)pentyl group, cyclohexyl group, indenyl group, furanyl group, thiophenyl group, pyrimidyl group, benzofuranyl group, benzimidazolyl group, quinolyl group, isoquinolyl group, quinoxalyl group, benzooxadiazolyl group, benzothiadiazolyl group, indolyl group, N-methylindolyl group, benzothiazolyl group, benzothiophenyl group or benzoisooxazolyl group, substituted naphthyl group,
when m is 0 and A is a substituted or non-substituted phenylene group, indenylene group or naphthylene group, or a substituted or non-substituted pyridylene group, furanylene group, thiophenylene group, pyrimidylene group, benzophenylene group, benzimidazolene group, quinolylene group, indolene group or benzothiazolene group, J represents a substituted or non-substituted cyclohexyl group, phenyl group, indenyl group, naphthyl group, furanyl group, thiophenyl group, pyrimidyl group, benzofuranyl group, benzimidazolyl group, quinolyl group, isoquinolyl group, quinoxalyl group, benzooxadiazolyl group, benzothiadiazolyl group, indolyl group, N-methylindolyl group, benzothiazolyl group, benzothiophenyl group or benzoisooxazolyl group;
when m is 0 and A is a single bond or when m is 1 or 2, J represents a substituted or non-substituted cyclohexyl group, phenyl group, indenyl group, naphthyl group, furanyl group, thiophenyl group, pyrimidyl group, benzofuranyl group, benzimidazolyl group, quinolyl group, isoquinolyl group, quinoxalyl group, benzooxadiazolyl group, benzothiadiazolyl group, indolyl group, N-methylindolyl group, benzothiazolyl group, benzothiophenyl group or benzoisooxazolyl group; substitution groups here are represented by a halogen atom, OH, NO₂, CN, methyl group, ethyl group, (n- or i-)propyl group, (n-, i-, s- or t-)butyl group, methoxy group, ethoxy group, (n- or i-)propyloxy group, (n-, i-, s- or t-)butyloxy group, methylthio group, ethylthio group, (n- or i-)propylthio group, (n-, i-, s- or t-)butylthio group, methylsulfonyl group, ethylsulfonyl group, (n- or i-)propylsulfonyl group, (n-, i-, s- or t-)butylsulfonyl group, acetyl group, ethylcarbonyl group, (n- or i-)propylcarbonyl group, acetylamino group, ethylcarbonylamino group, (n- or i-)propylcarbonylamino group, (n-, i-, s- or t-)butylcarbonylainino group, trifluoromethyl group or trifluoromethoxy group, and one or a plurality of these may be respectively and independently substituted at an arbitrary location of a ring or alkyl group; and,
X respresents CH or a nitrogen atom.

2. The thiobenzimidazole compound or medically acceptable salt thereof represented by the following formula (I), wherein,
R¹ and R² simultaneously or respectively independently represent a hydrogen atom, fluorine atom, chlorine atom, bromine atom, iodine atom, trifluoromethyl group, cyano group, hydroxyl group, methyl group, ethyl group, (n- or i-)propyl group, (n-, i-, s- or t-)butyl group, methoxy group, ethoxy group, (n- or i-)propyloxy group, (n-, i-, s- or t-)butyloxy group, or R¹ and R² together represent —O—CH₂—O—, —O—CH₂—CH₂—O— or —CH₂—CH₂—CH₂— in tis case, the carbon atoms may be substituted with one or a plurality of methyl groups, ethyl groups, (n- or i-)propyl groups or (n-, i-, s- or t-)butyl groups;
A represents a substituted or non-substituted methylene group, ethylene group, (n- or i-)propylene group or (n-, i- or t-)butylenes group, and substitution groups here are represented by a fluorine atom, chlorine atom, bromine atom, iodine atom, OH, NO₂, CN, methyl group, ethyl group, (n- or i-)propyl group, (n-, i-, or t-)butyl group, methoxy group, ethoxy group, (n- or i-)propyloxy group, (n-, i-, s- or t-)butyloxy group, in this case, substitution groups may be acetal-bonded at mutually adjacent sites, methylthio group, ethylthio group, (n- or i-)propylthio group, (n-, i-, s- or t-)butylthio group, methylsulfonyl group, ethylsulfonyl group, (n- or i-)propylsulfonyl group, (n-, i-, s- or t-)butylsulfonyl group, acetyl group, ethylcarbonyl group, (n- or i-)propylcarbonyl group, acetylamino group, ethylcarbonylaminio group, (n- or i-) propylcarbonylamino group, (n-, i-, s-, or t-)butylcarbonylamino group, trifluoromethyl group or trifluoromethoxy group, and one or a plurality of these may be respectively and independently substituted at an arbitrary location of alkylene group;
E represents COOR³, SO₃R³, CONHR³, SO₂NHR³, tetrazole-5-yl group, 5-oxo-1,2,4-oxadiazole-3-yl group or 5-oxo-1,2,4-thiadiazole-3-yl group wherein R represents a hydrogen atom, methyl group, ethyl group, (n- or i-)propyl group or (n-, i-, s- or t-)butyl group;
G represents a substituted or non-substituted methylene group, ethylene group, (n- or i-)propylene group or (n-, i- or t-)butylenes group, and one or a plurality of O, S, SO² or NR³ may be intermediately contained therein, where R³ is the same as previously defined, and substitution groups here are represented by a fluorine atom, chlorine atom, bromine atom, iodine atom, OH, NO₂, CN, methyl group, ethyl group, (n- or i-)propyl group, (n-, i-, s- or t-) butyl group, methoxy group, ethoxy group, (n- or i-)propyloxy group, (n-, i-, s- or t-)butyloxy group, trifluoromethyl group, trifluoromethoxy group or oxo group;
m represents an integer of 0-2;
J represents a substituted or non-substituted furanyl group, thiophenyl group, pyrimidyl group, benzofuranyl group, benzimidazolyl group, quinolyl group, isoquinolyl group, quinoxalyl group, benzooxadiazolyl group, benzothiadiazolyl group, indolyl group, benzothiazolyl group, benzothiophenyl group or benzoisooxazolyl group, substitution groups here are represented by a fluorine group, chlorine group, bromine group, iodine group, OH, NO₂, CN, methyl group, ethyl group, (n- or i-)propyl group, (n-, i-, s- or t-)butyl group, methoxy group, ethoxy group, (n- or i-)propyloxy group, (n-, i-, s- or t-)butyloxy group, methylthio group, ethylthio group, (n- or i-)propylthio group, (n-, i-, s- or t-)buthylthio group, methylsulfonyl group, ethylsulfonyl group, (n- or i-)propylsulfonyl group, (n-, i-, s- or t-) butylsulfonyl group, acetyl group, ethylcarbonyl group, (n- or i-)propylcarbonyl group, acetylamino group, ethylcarbonylamino group, (n- or i-)propylcarbonylamino group, (n-, i-, s- or t-)butylcarbonylamino group, trifluoromethyl group or trifluoromethoxy group, and one or a plurality of these may be respectively and independently substituted at an arbitrary location of a ring; and
X represents CH or a nitrogen atom.

3. The thiobenzimidazole compound or medically acceptable salt thereof according to claim 1, wherein, in the above formula (1), A is a substituted or non-substituted methylene, ethylene group, (n- or i-)propylene group or (n-, i- or t-)butylene group, a substituted or non-substituted phenylene group, indenylene group, naphthylene group, or a substituted or non-substituted pyridylene group, furanylene group, thiophenylene group, pyrimidylene group, benzophenylene group, benzimidazolene group, quinolylene group, indolene group or benzothiazolene group.

4. The thiobenzimidazole compound or medically acceptable salt thereof according to claim 1, wherein in the above formula (1), A is a substituted or non-substituted pyridylene group, furanylene group, thiophenylene group, pyrimidylene group, benzophenylen group, benzimidazolene group, quinolylene group, indolene group or benzothiazolene group.

5. The thiobenzimidazole compound or medically acceptable salt thereof according to claim 1, wherein in the formula (1), A is a substituted or non-substituted ethylene group.

6. The thiobenzimidazole compound or medically acceptable salt thereof according to claim 1 wherein, in the above formula (1), m is 1.

7. The thiobenzimidazole compound or medically acceptable salt thereof according to claim 1, wherein, in the above formula (1), m is 2.

8. The thiobenzimidazole compound or medically acceptable salt thereof according to claim 1, wherein, in the above formula (1), m is 0, A is a substituted or non-substituted methylene group, ethylene group, (n- or i-)propylene group or (n-, i- or t-) butylene group, and J is a substituted or non-substituted indenyl group or substituted naphthyl group.

9. The thiobenzimidazole compound or medically acceptable salt thereof according to claim 1, wherein, in the above formula (1), m is 0, A is a substituted or non-substituted methylene group, ethylene group, (n- or i-)propylene group or (n-, i- or t-)butylene group, and J is a substituted or non-substituted furanyl group, thiophenyl group, pyrimidyl group, benzofuranyl group, benzimidazolyl group, quinolyl group, isoquinolyl group, quinoxalyl group, benzooxadiazolyl group, benzothiadiazolyl group, indolyl group, N-methylindolyl group, benzothiazolyl group, benzothiophenyl group or benzoisooxazolyl group.

10. The thiobenzimidazole compound or medically acceptable salt thereof according to claim 1, wherein, in the above formula (1), m is 0, A is a substituted or non-substituted phenylene group, indenylene group or naphthylene group, a substituted or non-substituted pyridylene group, furanylene group, thiophenylene group, pyrimidylene group, benzophenylene group, benzimidazolene group, quinolylene group, indolene group or benzothiazolene group, and J is a substituted or non-substituted phenyl group, indenyl group or naphthyl group, or a substituted or non-substituted furanyl group, thiophenyl group, pyrimidyl group, benzofuranyl group, benzimidazolyl group, quinolyl group, isoquinolyl group, quinoxalyl group, benzooxadiazolyl group, benzothiadiazolyl group, indolyl group, N-methylindolyl group, benzothiazolyl group, benzothiophenyl group or benzoisooxazolyl group.

11. The thiobenzimidazole compound or medically acceptable salt thereof according to claim 1, wherein in the above formula (1), J is a substituted or unsubstitute indolyl group or benzothiophenyl group.

12. A thiobenzimidazole compound or medically acceptable salt thereof according to claim 1, wherein, in the above formula (1), G is —CH₂, —CH₂CH₂—, —CH₂CO—, —CH₂CH₂O—, —CH₂CONH—, —CO—, —CH₂SO₂—, —CH₂S— or —CH₂CH₂S—.

13. The thiobenzimidazole compound or medically acceptable salt thereof according to claim 1, wherein, in the above formula (1), R¹ and R² are simultaneously a hydrogen atom, halogen atom, methyl group, ethyl group, (n- or i-) propyl group, (n-, i-, s- or t-)butyl group, methoxy group, ethoxy group, (n- or i-)propyloxy group or (n-, i-, s-, or t-)butyloxy group, or R¹ and R² are respectively and independently a hydrogen atom, halogen atom, methyl group, ethyl group, (n- or i-)propyl group, (n-, i-, s- or t-)butyl group, methoxy group, ethoxy group, (n- or i-)propyloxy group, (n-, i-, s-, or t-)butyloxy group, trifluoromethyl group, cyano group or hydroxyl group.

14. The thiobenzimidazole compound or medically acceptable salt thereof according to claim 1, wherein in the above formula (1), R¹ and R² simultaneously or respectively independently represent a hydrogen atom, fluorine atom, chlorine atom, methyl group, ethyl group, (n- or i-)propyl group, (n-, i- s- or t-)butyl group, methoxy group, ethoxy group, (n- or i-)propyloxy group, (n-, i-, s- or t-)butyloxy group, trifluoromethyl group, cyano group, or hydroxyl group.

15. The thiobenzimidazole compound or medically acceptable salt thereof according to claim 1, wherein, in the above formula (1), E is COOH or a tetrazole group.

16. The thiobenzimidazole compound or medically acceptable salt thereof according claim 1, wherein, m in the above formula (1), X is CH.

17. A pharmaceutical composition comprising at least one thiobenzimidazole compound or medically acceptable salt thereof according to claim 1, and a pharmaceutically acceptable carrier.

18. A method for inhibiting human chymase by administering to a human subject an effective amount of a pharmaceutical composition comprising a thiobenzimidazole compound according to claim 1 as the active ingredient and a pharmaceticually acceptable carrier.

19. A method for inhibiting human chymase by administering to a human subject an effective amount of a pharmaceutical composition comprising a thiobenzimidazole compound according to claim 9 as the active ingredient and a pharmaceutically acceptable carrier.

20. A method for treating an allergic disease, bronchial asthma, cardiovascular disease selected from the group consisting of sclerosing vascular lesions, peripheral circulation disorders, renal insufficiency and cardiac insufficiency, and bone/cartilage metabolic diseases selected from the group consisting of rheumatoid arthritis and osteoarthritis by administering to a human subject an effective amount of a pharmaceutical composition comprising a thiobenzimidazole compound according to claim 1 as the active ingredient.

21. A method for treating an allergic disease, bronchial asthma, cardiovascular disease selected from the group consisting of sclerosing vascular lesions, peripheral circulation disorders, renal insufficiency and cardiac insufficiency, and bone/cartilage metabolic diseases selected from the group consisting of rheumatoid arthritis and osteoarthritis by administering to a human subject an effective amount of a pharmaceutical composition comprising a thiobenzimidazole compound according to claim 9 as the active ingredient.

### DETAILED DESCRIPTION OF THE INVENTION

### BEST MODE FOR CARRYING OUT THE INVENTION

The present invention will now be explained in more detail below.

The above definitions concerning the substituents of the compounds of formula (1) of the present invention are as follows:

R¹ and R², simultaneously or independently of each other, represent a hydrogen atom, a halogen atom, a trihalomethyl group, a cyano group, a hydroxy group, an alkyl group having 1 to 4 carbons or an alkoxy group having 1 to 4 carbons, or R¹ and R² together form —O—CH₂—O—, —O—CH₂—CH₂—O— or —CH₂—CH₂—CH₂—, in which the carbons may be substituted with one or a plurality of alkyl groups having 1 to 4 carbons. As the alkyl group having 1 to 4 carbons, there can be mentioned a methyl group, an ethyl group, a (n, i-) propyl group and a (n, i, s, t-) butyl group, and preferably a methyl group may be mentioned. Preferably R¹ and R² simultaneously represent a hydrogen atom, a halogen atom, an alkyl group having 1 to 4 carbons or an alkoxy group having 1 to 4 carbons, or R¹ and R², independently of each other, represent a hydrogen atom, a halogen atom, a trihalomethyl group, a cyano group, a hydroxy group, an alkyl group having 1 to 4 carbons, or an alkoxy group having 1 to 4 carbons. As the halogen atom, as used herein, there can be mentioned a fluorine atom, a chlorine atom, a bromine atom and the like, and preferably a chlorine atom and a fluorine atom may be mentioned. As the alkyl group having 1 to 4 carbons, there can be mentioned a methyl group, an ethyl group, a (n, i-) propyl group and a (n, i, t-) butyl group, and preferably a methyl group may be mentioned. As the alkoxy group having 1 to 4 carbons, there can be mentioned a methoxy group, an ethoxy group, a (n, i-) propyloxy group and a (n, i, s, t-) butyloxy group, and preferably a methoxy group may be mentioned.

A represents a substituted or unsubstituted, linear or branched alkylene group having 1 to 6 carbons, a substituted or unsubstituted arylene group having 6 to 11 carbons, or a substituted or unsubstituted heteroarylene group having 4 to 10 carbons that may contain one or a plurality of oxygen, nitrogen and sulfur atoms on the ring. Preferably, there can be mentioned a substituted or unsubstituted, linear or branched alkylene group having 1 to 6 carbons, a substituted or unsubstituted arylene group having 6 to 11 carbons, or a substituted or unsubstituted heteroarylene group having 4 to 10 carbons that may contain one or a plurality of oxygen, nitrogen and sulfur atoms on the ring. As the substituted or unsubstituted, linear or branched alkylene group having 1 to 6 carbons, there can be mentioned a methylene group, an ethylene group, a (n, i-) propylene group and a (n, i, t-) butylene group, and preferably an ethylene group may be mentioned. As the substituted or unsubstituted arylene group having 6 to 11 carbons, there can be mentioned a phenylene group, an indenylene group and a naphthylene group etc., and preferably a phenylene group may be mentioned. As the substituted or unsubstituted heteroarylene group having 4 to 10 carbons that may contain one or a plurality of oxygen, nitrogen and sulfur atoms on the ring, there can be mentioned a pyridilene group, a furanylene group, a thiophenylene group, an imidazolene group, a thiazolene group, a pyrimidilene group, an oxazolene group, an isoxazolene group, a benzphenylene group, a benzimidazolene group, a quinolilene group, an indolene group, a benzothiazolene group and the like, and preferably a pyridilene group, a furanylene group, and a thiophenylene group may be mentioned.

Furthermore, as the substituent, as used herein, there can be mentioned a halogen atom, OH, NO₂, CN, a linear or branched alkyl group having 1 to 6 carbons, a linear or branched alkoxy group having 1 to 6 carbons in which the substituent may be joined to each other at adjacent sites via an acetal bond, a linear or branched alkylthio group having 1 to 6 carbons, a linear or branched alkylsulfonyl group having 1 to 6 carbons, a linear or branched acyl group having 1 to 6 carbons, a linear or branched acylamino group having 1 to 6 carbons, a trihalomethyl group, a trihalomethoxy group, a phenyl group, or a phenoxy group that may be substituted with one or more halogen atoms. They may be independently substituted at any one or more sites of the ring or the alkylene group. Specifically, there can be mentioned OH, a chloro group, a bromo group, a nitro group, a methoxy group, a cyano group, a methylenedioxy group, a trifluoromethyl group, a methyl group, an ethyl group, a (n, i-) propyl group, a (n, i, t-) butyl group, and the like.

As E, there can be mentioned COOR³, SO₃R³, CONHR³, SO₂NHR³, a tetrazole group, a 5-oxo-1,2,4-oxadiazole group or a 5-oxo-1,2,4-thiadiazole group, and preferably COOR³ or a tetrazole group may be mentioned. As R³ as used herein, there can be mentioned a hydrogen atom or a linear or branched alkyl group having 1 to 6 carbons, and preferably a hydrogen atom, a methyl group, an ethyl group, or a t-butyl group may be mentioned, and most preferably a hydrogen atom may be mentioned.

G represents a substituted or unsubstituted, linear or branched alkylene group having 1 to 6 carbons that may be interrupted with one or a plurality of O, S, SO₂, and NR³, in which R³ is as defined above and the substituent represents a halogen atom, OH, NO₂, CN, a linear or branched alkyl group having 1 to 6 carbons, a linear or branched alkoxy group having 1 to 6 carbons (the substituents may be joined to each other at adjacent sites via an acetal bond), a trihalomethyl group, a trihalomethoxy group, a phenyl group, or an oxo group. Specifically, there can be mentioned —CH₂—, —CH₂CH₂—, —CH₂CO—, —CH₂CH₂O—, CH₂CONH—, —CO—, —SO₂—, —CH₂SO₂—, —CH₂S—, —CH₂CH₂S— and the like, and preferably —CH₂—, —CH₂CH₂—, —CH₂CO— or —CH₂CH₂O— may be mentioned.
m represents an integer of 0 to 2, and preferably 0 or 2 may be mentioned.

When m is 0 and A is a substituted or unsubstituted, linear or branched alkylene group having 1 to 6 carbons, then J represents a substituted or unsubstituted, linear, cyclic or branched alkyl group having 1 to 6 carbons, a substituted or unsubstituted aryl group having 7 to 9 carbons, a substituted aryl group having 10 to 11 carbons, a substituted or unsubstituted heteroaryl group having 4 to 10 carbons that may contain one or a plurality of oxygen, nitrogen and sulfur atoms on the ring. Preferably, a substituted aryl group having 10 to 11 carbons and a substituted or unsubstituted heteroaryl group having 4 to 10 carbons that may contain one or a plurality of oxygen, nitrogen and sulfur atoms on the ring may be mentioned. As the substituted or unsubstituted, linear, cyclic or branched alkyl group having 1 to 6 carbons, there can be mentioned a (n, i-) propyl group, a (n, i, s, t-) butyl group, a (n, i, ne, t-) pentyl group and a cyclohexyl group. As the substituted or unsubstituted aryl group having 7 to 9 carbons, there can be mentioned an indenyl group, and as the substituted aryl group having 10 to 11 carbons, there can be mentioned a naphthyl group. As the substituted or unsubstituted heteroaryl group having 4 to 10 carbons that may contain one or a plurality of oxygen, nitrogen and sulfur atoms on the ring, there can be mentioned a pyridyl group, a furanyl group, a thiophenyl group, an imidazole group, a thiazole group, a pyrimidine group, an oxazole group, an isoxazole group, a benzofurane group, a benzimidazole group, a quinoline group, an isoquinoline group, a quinoxaline group, a benzoxadiazole group, a benzothiadiazole group, an indole group, a N-methylindole group, a benzothiazole group, a benzothiophenyl group, a benzisoxazole group and the like, and preferably a benzothiophenyl group or a N-methylindole group may be mentioned.

When m is 0 and A is a substituted or unsubstituted arylene group having 6 to 11 carbons or a substituted or unsubstituted heteroarylene group having 4 to 10 carbons that may contain one or a plurality of oxygen, nitrogen and sulfur atoms on the ring, then J represents a substituted or unsubstituted, linear, cyclic or branched alkyl group having 1 to 6 carbons, a substituted or unsubstituted aryl group having 6 to 11 carbons, or a substituted or unsubstituted heteroaryl group having 4 to 10 carbons that may contain one or a plurality of oxygen, nitrogen and sulfur atoms on the ring, and preferably a substituted or unsubstituted aryl group having 6 to 11 carbons and a substituted or unsubstituted heteroaryl group having 4 to 10 carbons that may contain one or a plurality of oxygen, nitrogen and sulfur atoms on the ring may be mentioned. As the substituted or unsubstituted aryl group having 6 to 11 carbons, there can be mentioned a phenyl group, an indenyl group, a naphthyl group and the like, and preferably a phenyl group or a naphthyl group may be mentioned. As the substituted or unsubstituted, linear, cyclic or branched alkyl group having 1 to 6 carbons and as the substituted or unsubstituted heteroaryl group having 4 to 10 carbons that may contain one or a plurality of oxygen, nitrogen and sulfur atoms on the ring, there can be mentioned those described above. As the substituent as used herein, there can be mentioned a halogen atom, OH, NO₂, CN, a linear or branched alkyl group having 1 to 6 carbons, a linear or branched alkoxy group having 1 to 6 carbons (the substituents may be joined to each other at adjacent sites via an acetal bond), a linear or branched alkylthio group having 1 to 6 carbons, a linear or branched alkylsulfonyl group having 1 to 6 carbons, a linear or branched acyl group having 1 to 6 carbons, a linear or branched acylamino group having 1 to 6 carbons, a substituted or unsubstituted anilide group, a trihalomethyl group, a trihalomethoxy group, a phenyl group, or a phenoxy group that may be substituted with one or more halogen atoms. They may be independently substituted at any one or more sites of the ring or the alkyl group. Specifically, there can be mentioned OH, a chloro group, a bromo group, a nitro group, a methoxy group, a cyano group, a methylenedioxy group, a trifluoromethyl group, a trifluoromethoxy group, a methyl group, an ethyl group, a (n, i-) propyl group, a (n, i, s, t-) butyl group, an anilide group and the like.

X represents CH or a nitrogen atom, and preferably CH may be mentioned.

As the compound of formula (1), specifically those described in Tables 1 to 68 are preferred. Most preferred among them are compounds Nos. 37, 50, 63, 64, 65, 84, 115, 117, 119, 121, 123, 130, 143, 147, 168, 174, 256, 264, 272, 311, 319, 320, 321, 324, 349, 352, 354, 355, 358, 364, 380, 392, 395, 398, 401, 402, 444, 455, 456, 459, 460, 463, 471, 475, 491, 506, 863, 866, 869, 1026, 1027, 1029, 1030, 1039, 1041, 1043, 1044, 1048, 1112, 1114, 1126, 1128, 1382, 1458, 1460, 1470, 1472, 1474, 1544, 1645 and 1647.

A1 to A22 and J1 to J114 described in Tables 1 to 68 are the groups shown below, in which E and G are as described above.

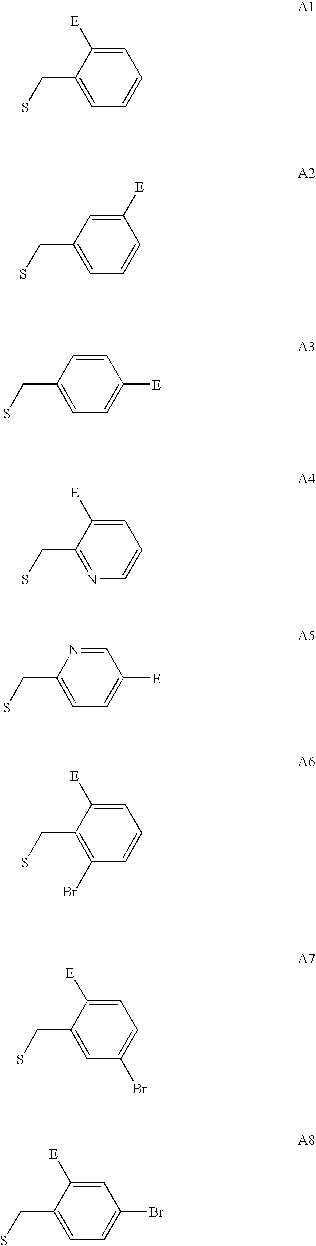

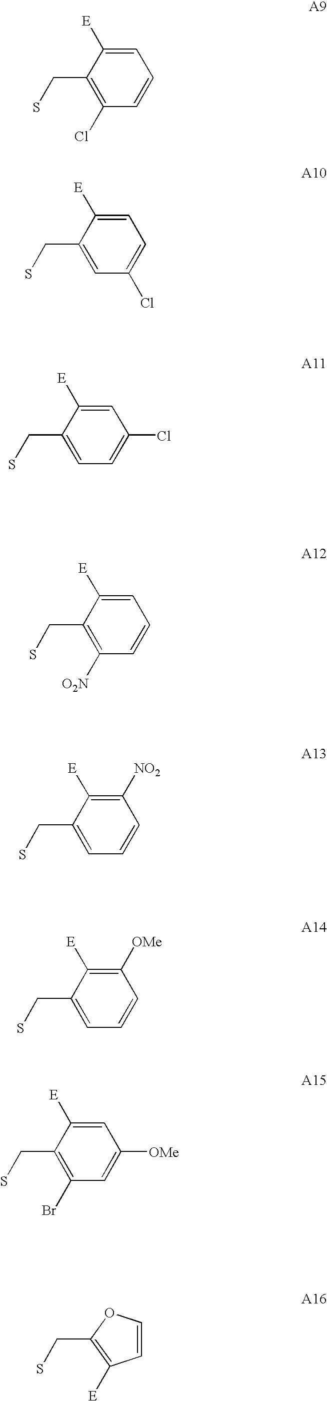

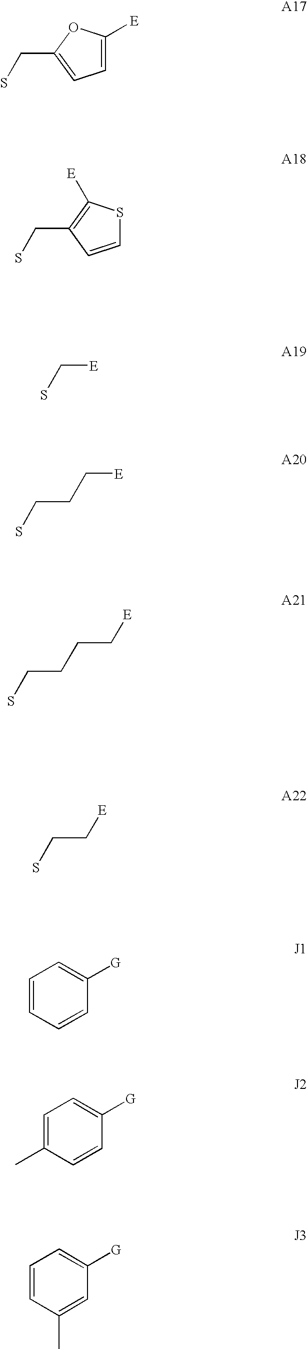

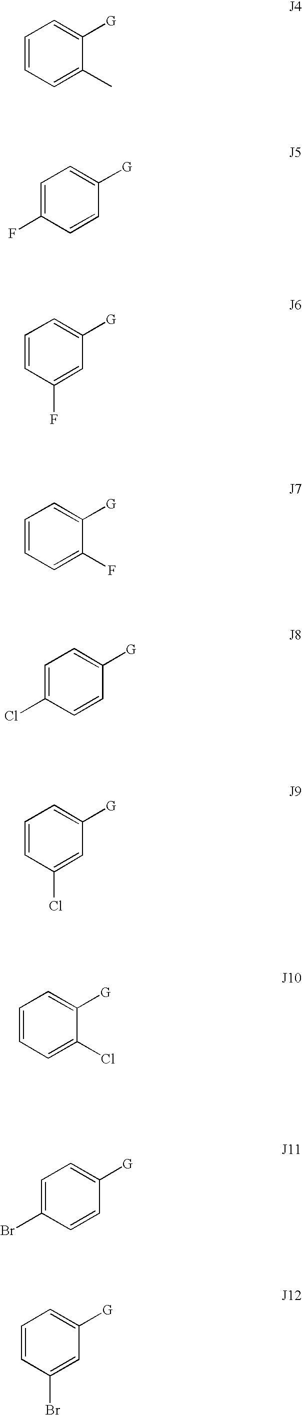

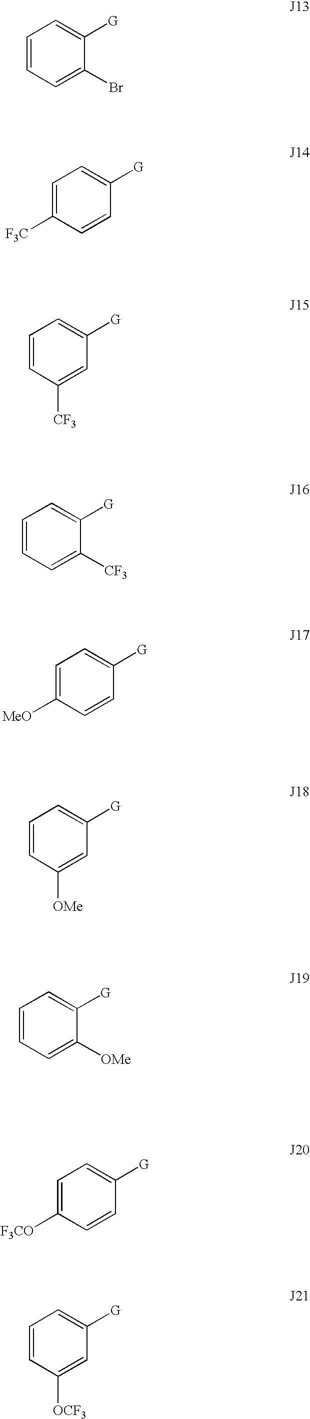

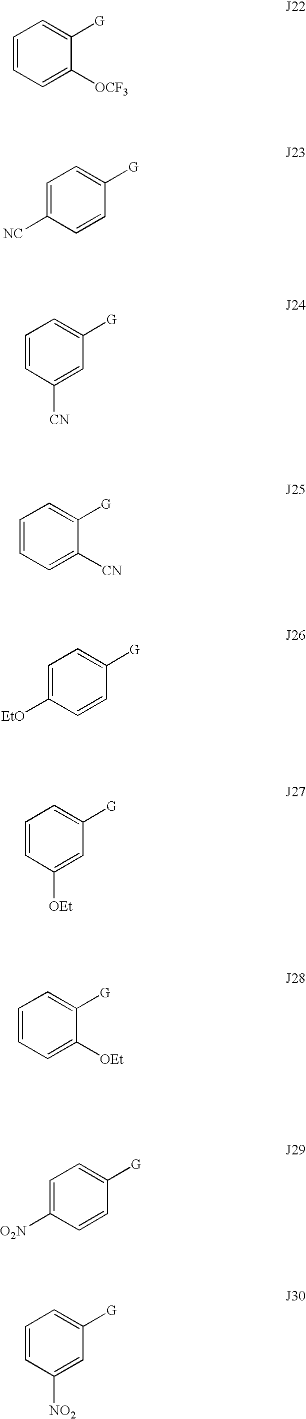

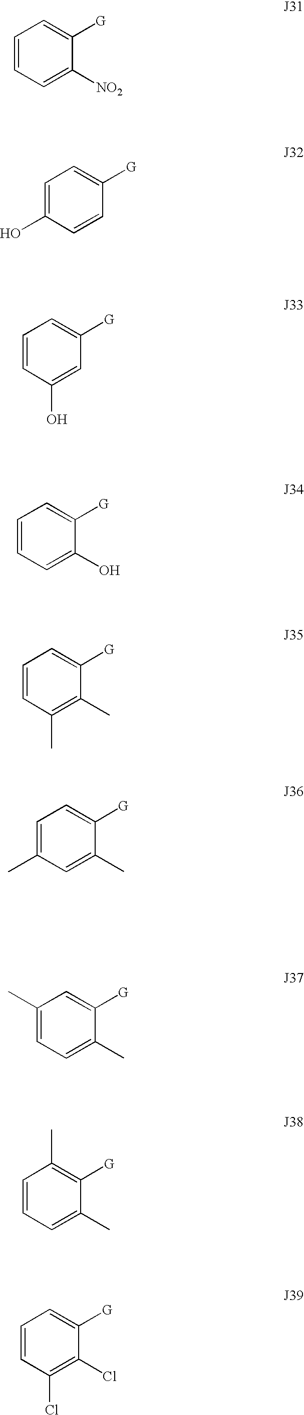

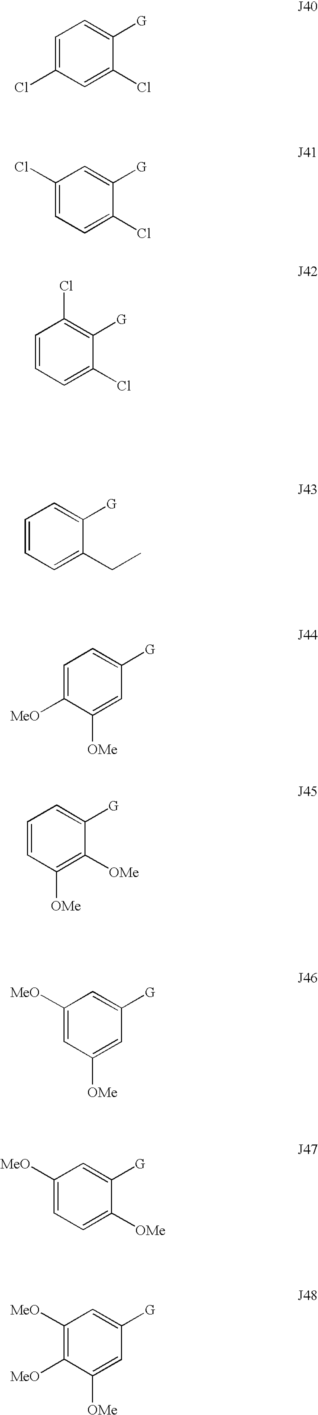

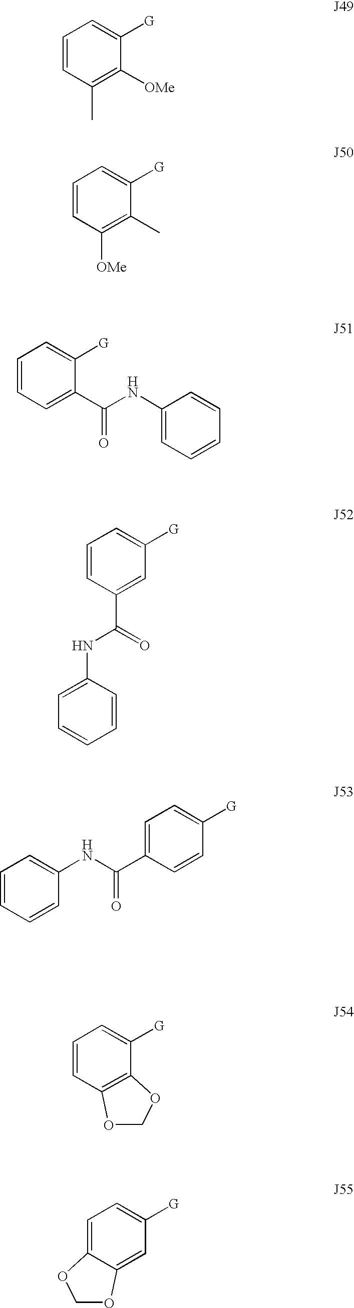

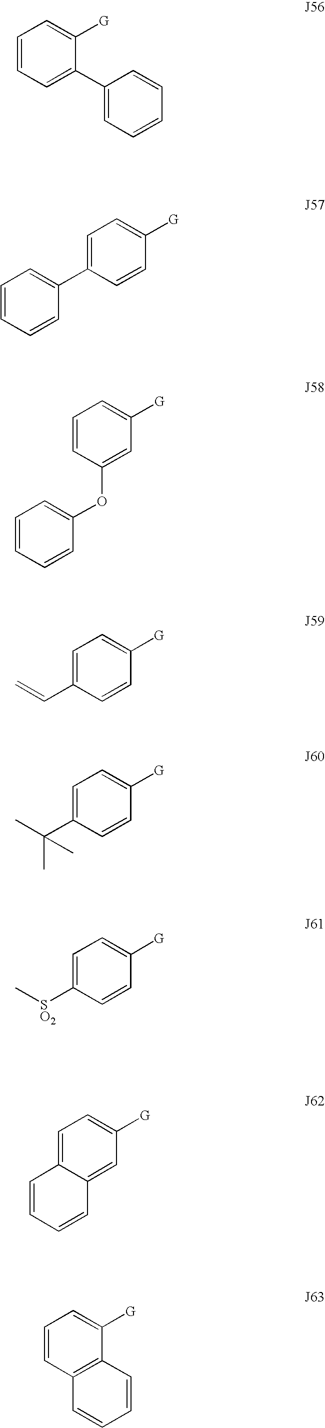

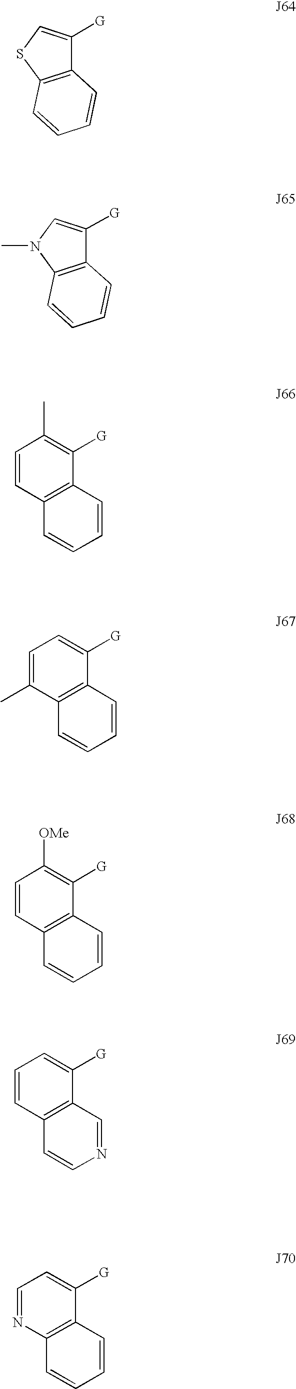

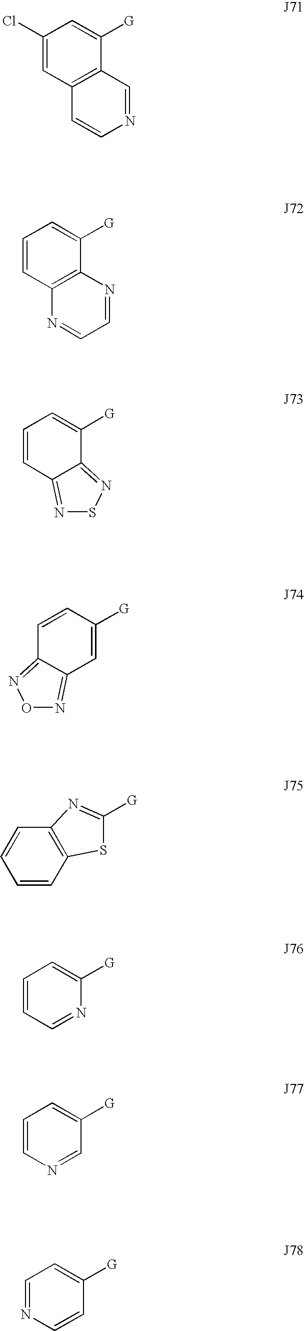

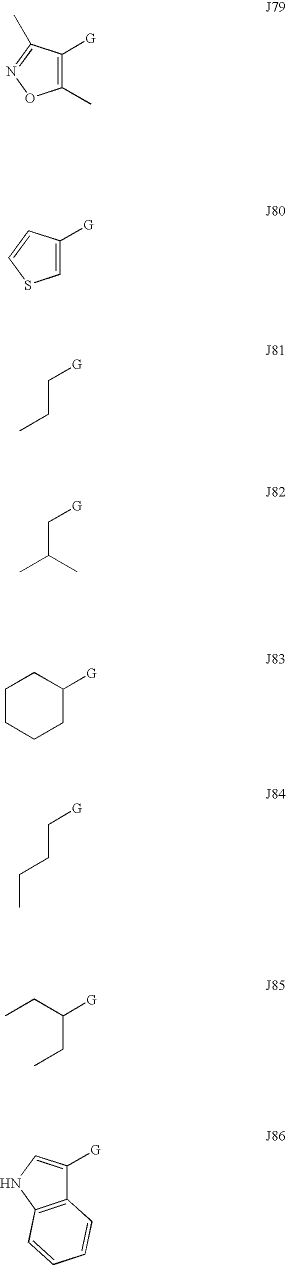

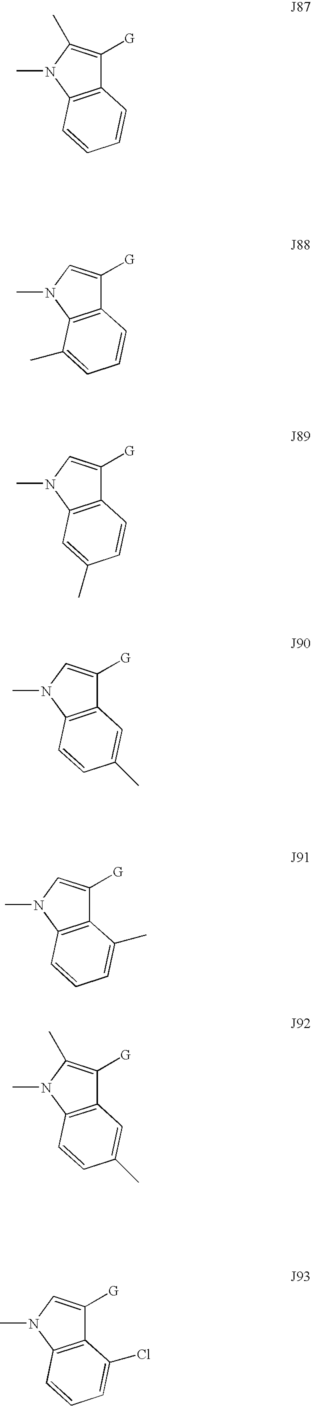

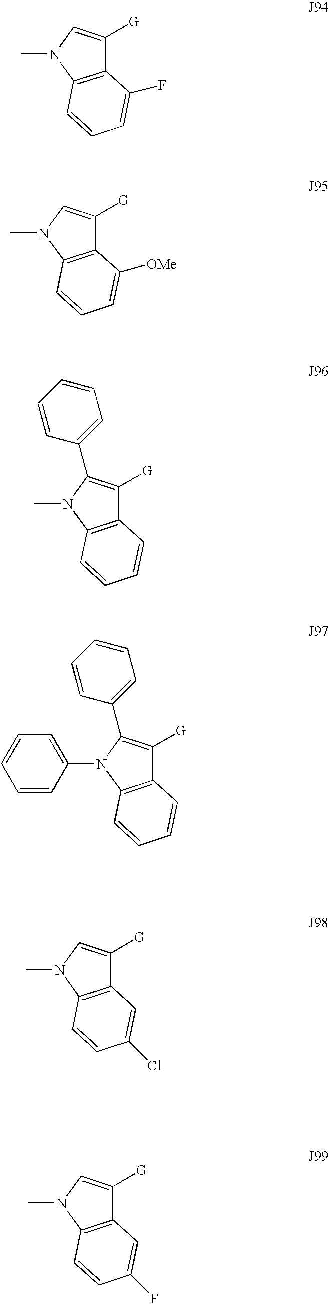

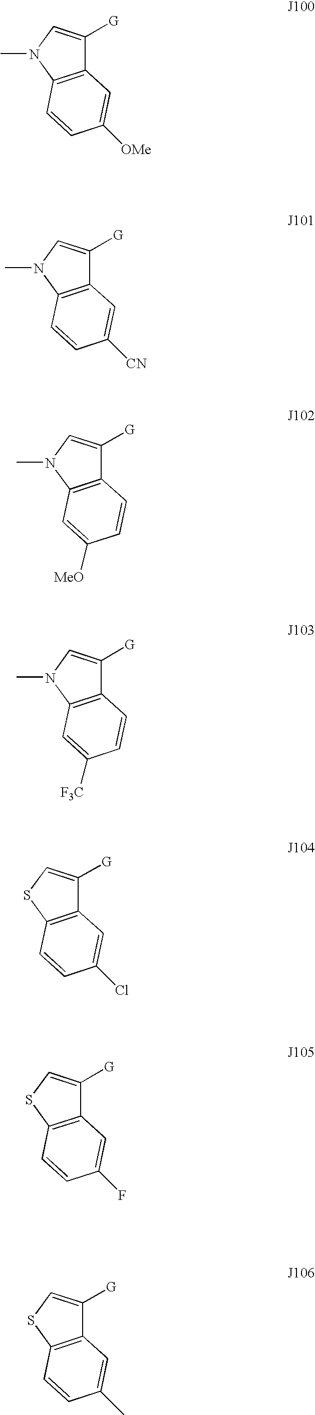

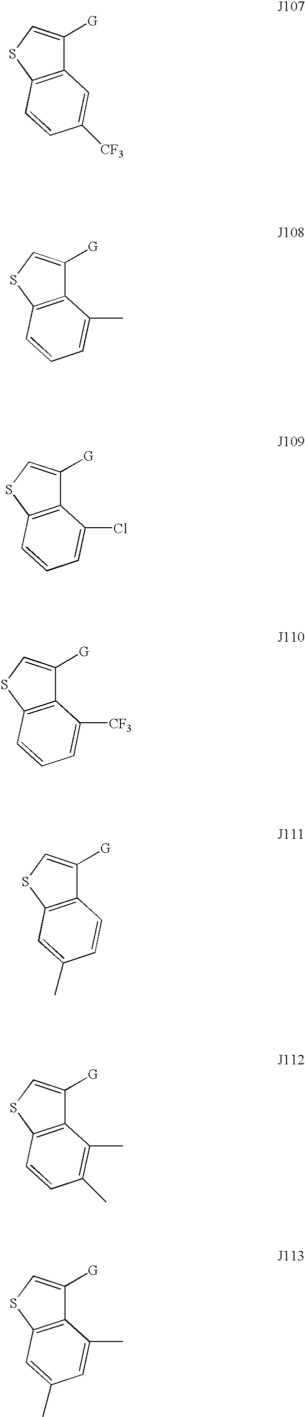

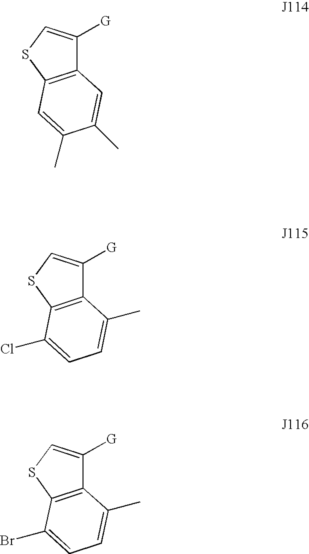

TABLE 1

| Compound No. | R¹ | R² | SCH₂-A | E | G | J | m | X |
|---|---|---|---|---|---|---|---|---|
| 1 | H | H | A1 | COOH | CH₂CH₂ | J1 | 0 | CH |
| 2 | H | H | A1 | COOH | CH₂ | J2 | 0 | CH |
| 3 | H | H | A1 | COOH | CH₂ | J3 | 0 | CH |
| 4 | H | H | A1 | COOH | CH₂ | J4 | 0 | CH |
| 5 | H | H | A1 | COOH | CH₂ | J5 | 0 | CH |
| 6 | H | H | A1 | COOH | CH₂ | J6 | 0 | CH |
| 7 | H | H | A1 | COOH | CH₂ | J7 | 0 | CH |
| 8 | H | H | A1 | COOH | CH₂ | J8 | 0 | CH |
| 9 | H | H | A1 | COOH | CH₂ | J9 | 0 | CH |
| 10 | H | H | A1 | COOH | CH₂ | J10 | 0 | CH |
| 11 | H | H | A1 | COOH | CH₂ | J11 | 0 | CH |
| 12 | H | H | A1 | COOH | CH₂ | J12 | 0 | CH |
| 13 | H | H | A1 | COOH | CH₂ | J13 | 0 | CH |
| 14 | H | H | A1 | COOH | CH₂ | J14 | 0 | CH |
| 15 | H | H | A1 | COOH | CH₂ | J15 | 0 | CH |
| 16 | H | H | A1 | COOH | CH₂ | J16 | 0 | CH |
| 17 | H | H | A1 | COOH | CH₂ | J17 | 0 | CH |
| 18 | H | H | A1 | COOH | CH₂ | J18 | 0 | CH |
| 19 | H | H | A1 | COOH | CH₂ | J19 | 0 | CH |
| 20 | H | H | A1 | COOH | CH₂ | J20 | 0 | CH |
| 21 | H | H | A1 | COOH | CH₂ | J21 | 0 | CH |
| 22 | H | H | A1 | COOH | CH₂ | J22 | 0 | CH |
| 23 | H | H | A1 | COOH | CH₂ | J23 | 0 | CH |
| 24 | H | H | A1 | COOH | CH₂ | J24 | 0 | CH |
| 25 | H | H | A1 | COOH | CH₂ | J25 | 0 | CH |

TABLE 2

| Compound No. | R¹ | R² | SCH₂-A | E | G | J | m | X |
|---|---|---|---|---|---|---|---|---|
| 26 | H | H | A1 | COOH | CH₂ | J26 | 0 | CH |
| 27 | H | H | A1 | COOH | CH₂ | J27 | 0 | CH |
| 28 | H | H | A1 | COOH | CH₂ | J28 | 0 | CH |
| 29 | H | H | A1 | COOH | CH₂ | J29 | 0 | CH |
| 30 | H | H | A1 | COOH | CH₂ | J30 | 0 | CH |
| 31 | H | H | A1 | COOH | CH₂ | J31 | 0 | CH |
| 32 | H | H | A1 | COOH | CH₂ | J32 | 0 | CH |
| 33 | H | H | A1 | COOH | CH₂ | J33 | 0 | CH |
| 34 | H | H | A1 | COOH | CH₂ | J34 | 0 | CH |
| 35 | H | H | A1 | COOH | CH₂ | J35 | 0 | CH |
| 36 | H | H | A1 | COOH | CH₂ | J36 | 0 | CH |
| 37 | H | H | A1 | COOH | CH₂ | J37 | 0 | CH |
| 38 | H | H | A1 | COOH | CH₂ | J38 | 0 | CH |
| 39 | H | H | A1 | COOH | CH₂ | J39 | 0 | CH |
| 40 | H | H | A1 | COOH | CH₂ | J40 | 0 | CH |
| 41 | H | H | A1 | COOH | CH₂ | J41 | 0 | CH |
| 42 | H | H | A1 | COOH | CH₂ | J42 | 0 | CH |
| 43 | H | H | A1 | COOH | CH₂ | J43 | 0 | CH |
| 44 | H | H | A1 | COOH | CH₂ | J44 | 0 | CH |
| 45 | H | H | A1 | COOH | CH₂ | J45 | 0 | CH |
| 46 | H | H | A1 | COOH | CH₂ | J46 | 0 | CH |
| 47 | H | H | A1 | COOH | CH₂ | J47 | 0 | CH |
| 48 | H | H | A1 | COOH | CH₂ | J48 | 0 | CH |
| 49 | H | H | A1 | COOH | CH₂ | J49 | 0 | CH |
| 50 | H | H | A1 | COOH | CH₂ | J50 | 0 | CH |

TABLE 3

| Compound No. | R¹ | R² | SCH₂-A | E | G | J | m | X |
|---|---|---|---|---|---|---|---|---|
| 51 | H | H | A1 | COOH | CH₂ | J51 | 0 | CH |
| 52 | H | H | A1 | COOH | CH₂ | J52 | 0 | CH |
| 53 | H | H | A1 | COOH | CH₂ | J53 | 0 | CH |
| 54 | H | H | A1 | COOH | CH₂ | J54 | 0 | CH |
| 55 | H | H | A1 | COOH | CH₂ | J55 | 0 | CH |
| 56 | H | H | A1 | COOH | CH₂ | J56 | 0 | CH |
| 57 | H | H | A1 | COOH | CH₂ | J57 | 0 | CH |
| 58 | H | H | A1 | COOH | CH₂ | J58 | 0 | CH |
| 59 | H | H | A1 | COOH | CH₂ | J59 | 0 | CH |
| 60 | H | H | A1 | COOH | CH₂ | J60 | 0 | CH |
| 61 | H | H | A1 | COOH | CH₂ | J61 | 0 | CH |
| 62 | H | H | A1 | COOH | CH₂ | J62 | 0 | CH |
| 63 | H | H | A1 | COOH | CH₂ | J63 | 0 | CH |
| 64 | H | H | A1 | COOH | CH₂ | J64 | 0 | CH |
| 65 | H | H | A1 | COOH | CH₂ | J65 | 0 | CH |
| 66 | H | H | A1 | COOH | CH₂ | J66 | 0 | CH |
| 67 | H | H | A1 | COOH | CH₂ | J67 | 0 | CH |
| 68 | H | H | A1 | COOH | CH₂ | J68 | 0 | CH |
| 69 | H | H | A1 | COOH | CH₂ | J69 | 0 | CH |
| 70 | H | H | A1 | COOH | CH₂ | J70 | 0 | CH |
| 71 | H | H | A1 | COOH | CH₂ | J71 | 0 | CH |
| 72 | H | H | A1 | COOH | CH₂ | J72 | 0 | CH |
| 73 | H | H | A1 | COOH | CH₂ | J73 | 0 | CH |
| 74 | H | H | A1 | COOH | CH₂ | J74 | 0 | CH |
| 75 | H | H | A1 | COOH | CH₂ | J75 | 0 | CH |

TABLE 4

| Compound No. | R¹ | R² | SCH₂-A | E | G | J | m | X |
|---|---|---|---|---|---|---|---|---|
| 76 | H | H | A1 | COOH | CH₂ | J76 | 0 | CH |
| 77 | H | H | A1 | COOH | CH₂ | J77 | 0 | CH |
| 78 | H | H | A1 | COOH | CH₂ | J78 | 0 | CH |
| 79 | H | H | A1 | COOH | CH₂ | J79 | 0 | CH |
| 80 | H | H | A1 | COOH | CH₂ | J80 | 0 | CH |
| 81 | Me | Me | A1 | COOH | CH₂ | J1 | 0 | CH |
| 82 | Me | Me | A1 | COOH | CH₂ | J2 | 0 | CH |
| 83 | Me | Me | A1 | COOH | CH₂ | J3 | 0 | CH |
| 84 | Me | Me | A1 | COOH | CH₂ | J4 | 0 | CH |
| 85 | Me | Me | A1 | COOH | CH₂ | J5 | 0 | CH |
| 86 | Me | Me | A1 | COOH | CH₂ | J6 | 0 | CH |
| 87 | Me | Me | A1 | COOH | CH₂ | J7 | 0 | CH |
| 88 | Me | Me | A1 | COOH | CH₂ | J8 | 0 | CH |
| 89 | Me | Me | A1 | COOH | CH₂ | J9 | 0 | CH |
| 90 | Me | Me | A1 | COOH | CH₂ | J10 | 0 | CH |
| 91 | Me | Me | A1 | COOH | CH₂ | J11 | 0 | CH |
| 92 | Me | Me | A1 | COOH | CH₂ | J12 | 0 | CH |
| 93 | Me | Me | A1 | COOH | CH₂ | J13 | 0 | CH |
| 94 | Me | Me | A1 | COOH | CH₂ | J14 | 0 | CH |
| 95 | Me | Me | A1 | COOH | CH₂ | J15 | 0 | CH |
| 96 | Me | Me | A1 | COOH | CH₂ | J16 | 0 | CH |
| 97 | Me | Me | A1 | COOH | CH₂ | J17 | 0 | CH |
| 98 | Me | Me | A1 | COOH | CH₂ | J18 | 0 | CH |
| 99 | Me | Me | A1 | COOH | CH₂ | J19 | 0 | CH |
| 100 | Me | Me | A1 | COOH | CH₂ | J20 | 0 | CH |

TABLE 5

| Compound No. | R¹ | R² | SCH₂-A | E | G | J | m | X |
|---|---|---|---|---|---|---|---|---|
| 101 | Me | Me | A1 | COOH | CH₂ | J21 | 0 | CH |
| 102 | Me | Me | A1 | COOH | CH₂ | J22 | 0 | CH |
| 103 | Me | Me | A1 | COOH | CH₂ | J23 | 0 | CH |
| 104 | Me | Me | A1 | COOH | CH₂ | J24 | 0 | CH |
| 105 | Me | Me | A1 | COOH | CH₂ | J25 | 0 | CH |
| 106 | Me | Me | A1 | COOH | CH₂ | J26 | 0 | CH |
| 107 | Me | Me | A1 | COOH | CH₂ | J27 | 0 | CH |
| 108 | Me | Me | A1 | COOH | CH₂ | J28 | 0 | CH |
| 109 | Me | Me | A1 | COOH | CH₂ | J29 | 0 | CH |
| 110 | Me | Me | A1 | COOH | CH₂ | J30 | 0 | CH |
| 111 | Me | Me | A1 | COOH | CH₂ | J31 | 0 | CH |
| 112 | Me | Me | A1 | COOH | CH₂ | J32 | 0 | CH |
| 113 | Me | Me | A1 | COOH | CH₂ | J33 | 0 | CH |
| 114 | Me | Me | A1 | COOH | CH₂ | J34 | 0 | CH |
| 115 | Me | Me | A1 | COOH | CH₂ | J35 | 0 | CH |
| 116 | Me | Me | A1 | COOH | CH₂ | J36 | 0 | CH |
| 117 | Me | Me | A1 | COOH | CH₂ | J37 | 0 | CH |
| 118 | Me | Me | A1 | COOH | CH₂ | J38 | 0 | CH |
| 119 | Me | Me | A1 | COOH | CH₂ | J39 | 0 | CH |
| 120 | Me | Me | A1 | COOH | CH₂ | J40 | 0 | CH |
| 121 | Me | Me | A1 | COOH | CH₂ | J41 | 0 | CH |
| 122 | Me | Me | A1 | COOH | CH₂ | J42 | 0 | CH |
| 123 | Me | Me | A1 | COOH | CH₂ | J43 | 0 | CH |
| 124 | Me | Me | A1 | COOH | CH₂ | J44 | 0 | CH |
| 125 | Me | Me | A1 | COOH | CH₂ | J45 | 0 | CH |

TABLE 6

| Compound No. | R¹ | R² | SCH₂-A | E | G | J | m | X |
|---|---|---|---|---|---|---|---|---|
| 126 | Me | Me | A1 | COOH | CH₂ | J46 | 0 | CH |
| 127 | Me | Me | A1 | COOH | CH₂ | J47 | 0 | CH |
| 128 | Me | Me | A1 | COOH | CH₂ | J48 | 0 | CH |
| 129 | Me | Me | A1 | COOH | CH₂ | J49 | 0 | CH |
| 130 | Me | Me | A1 | COOH | CH₂ | J50 | 0 | CH |
| 131 | Me | Me | A1 | COOH | CH₂ | J51 | 0 | CH |
| 132 | Me | Me | A1 | COOH | CH₂ | J52 | 0 | CH |
| 133 | Me | Me | A1 | COOH | CH₂ | J53 | 0 | CH |
| 134 | Me | Me | A1 | COOH | CH₂ | J54 | 0 | CH |
| 135 | Me | Me | A1 | COOH | CH₂ | J55 | 0 | CH |
| 136 | Me | Me | A1 | COOH | CH₂ | J56 | 0 | CH |
| 137 | Me | Me | A1 | COOH | CH₂ | J57 | 0 | CH |
| 138 | Me | Me | A1 | COOH | CH₂ | J58 | 0 | CH |
| 139 | Me | Me | A1 | COOH | CH₂ | J59 | 0 | CH |
| 140 | Me | Me | A1 | COOH | CH₂ | J60 | 0 | CH |
| 141 | Me | Me | A1 | COOH | CH₂ | J61 | 0 | CH |
| 142 | Me | Me | A1 | COOH | CH₂ | J62 | 0 | CH |
| 143 | Me | Me | A1 | COOH | CH₂ | J63 | 0 | CH |
| 144 | Me | Me | A1 | COOH | CH₂ | J64 | 0 | CH |
| 145 | Me | Me | A1 | COOH | CH₂ | J65 | 0 | CH |
| 146 | Me | Me | A1 | COOH | CH₂ | J66 | 0 | CH |
| 147 | Me | Me | A1 | COOH | CH₂ | J67 | 0 | CH |
| 148 | Me | Me | A1 | COOH | CH₂ | J68 | 0 | CH |
| 149 | Me | Me | A1 | COOH | CH₂ | J69 | 0 | CH |
| 150 | Me | Me | A1 | COOH | CH₂ | J70 | 0 | CH |

TABLE 7

| Compound No. | R¹ | R² | SCH₂-A | E | G | J | m | X |
|---|---|---|---|---|---|---|---|---|
| 151 | Me | Me | A1 | COOH | CH₂ | J71 | 0 | CH |
| 152 | Me | Me | A1 | COOH | CH₂ | J72 | 0 | CH |
| 153 | Me | Me | A1 | COOH | CH₂ | J73 | 0 | CH |
| 154 | Me | Me | A1 | COOH | CH₂ | J74 | 0 | CH |
| 155 | Me | Me | A1 | COOH | CH₂ | J75 | 0 | CH |
| 156 | Me | Me | A1 | COOH | CH₂ | J76 | 0 | CH |
| 157 | Me | Me | A1 | COOH | CH₂ | J77 | 0 | CH |
| 158 | Me | Me | A1 | COOH | CH₂ | J78 | 0 | CH |
| 159 | Me | Me | A1 | COOH | CH₂ | J79 | 0 | CH |
| 160 | Me | Me | A1 | COOH | CH₂ | J80 | 0 | CH |
| 161 | Cl | Cl | A1 | COOH | CH₂CH₂ | J1 | 0 | CH |
| 162 | Cl | Cl | A1 | COOH | CH₂ | J4 | 0 | CH |
| 163 | Cl | Cl | A1 | COOH | CH₂ | J10 | 0 | CH |
| 164 | Cl | Cl | A1 | COOH | CH₂ | J18 | 0 | CH |
| 165 | Cl | Cl | A1 | COOH | CH₂ | J21 | 0 | CH |
| 166 | Cl | Cl | A1 | COOH | CH₂ | J28 | 0 | CH |
| 167 | Cl | Cl | A1 | COOH | CH₂ | J35 | 0 | CH |
| 168 | Cl | Cl | A1 | COOH | CH₂ | J37 | 0 | CH |
| 169 | Cl | Cl | A1 | COOH | CH₂ | J39 | 0 | CH |
| 170 | Cl | Cl | A1 | COOH | CH₂ | J43 | 0 | CH |
| 171 | Cl | Cl | A1 | COOH | CH₂ | J46 | 0 | CH |
| 172 | Cl | Cl | A1 | COOH | CH₂ | J50 | 0 | CH |
| 173 | Cl | Cl | A1 | COOH | CH₂ | J54 | 0 | CH |
| 174 | Cl | Cl | A1 | COOH | CH₂ | J63 | 0 | CH |
| 175 | Cl | Cl | A1 | COOH | CH₂ | J64 | 0 | CH |

TABLE 8

| Com- | | | | | | | | |
|---|---|---|---|---|---|---|---|---|
| pound | | | | | | | | |
| No. | R¹ | R² | SCH₂-A | E | G | J | m | X |
| 176 | Cl | Cl | A1 | COOH | CH₂ | J65 | 0 | CH |
| 177 | Cl | Cl | A1 | COOH | CH₂ | J66 | 0 | CH |
| 178 | Cl | Cl | A1 | COOH | CH₂ | J67 | 0 | CH |
| 179 | Cl | Cl | A1 | COOH | CH₂ | J71 | 0 | CH |

| | | | | | | | |
|---|---|---|---|---|---|---|---|
| 180 | —CH₂CH₂CH₂— | A1 | COOH | CH₂CH₂ | J1 | 0 | CH |
| 181 | —CH₂CH₂CH₂— | A1 | COOH | CH₂ | J4 | 0 | CH |
| 182 | —CH₂CH₂CH₂— | A1 | COOH | CH₂ | J10 | 0 | CH |
| 183 | —CH₂CH₂CH₂— | A1 | COOH | CH₂ | J18 | 0 | CH |
| 184 | —CH₂CH₂CH₂— | A1 | COOH | CH₂ | J21 | 0 | CH |
| 185 | —CH₂CH₂CH₂— | A1 | COOH | CH₂ | J28 | 0 | CH |
| 186 | —CH₂CH₂CH₂— | A1 | COOH | CH₂ | J35 | 0 | CH |
| 187 | —CH₂CH₂CH₂— | A1 | COOH | CH₂ | J37 | 0 | CH |
| 188 | —CH₂CH₂CH₂— | A1 | COOH | CH₂ | J39 | 0 | CH |
| 189 | —CH₂CH₂CH₂— | A1 | COOH | CH₂ | J43 | 0 | CH |
| 190 | —CH₂CH₂CH₂— | A1 | COOH | CH₂ | J46 | 0 | CH |
| 191 | —CH₂CH₂CH₂— | A1 | COOH | CH₂ | J50 | 0 | CH |
| 192 | —CH₂CH₂CH₂— | A1 | COOH | CH₂ | J54 | 0 | CH |
| 193 | —CH₂CH₂CH₂— | A1 | COOH | CH₂ | J63 | 0 | CH |
| 194 | —CH₂CH₂CH₂— | A1 | COOH | CH₂ | J64 | 0 | CH |
| 195 | —CH₂CH₂CH₂— | A1 | COOH | CH₂ | J65 | 0 | CH |
| 196 | —CH₂CH₂CH₂— | A1 | COOH | CH₂ | J66 | 0 | CH |
| 197 | —CH₂CH₂CH₂— | A1 | COOH | CH₂ | J67 | 0 | CH |
| 198 | —CH₂CH₂CH₂— | A1 | COOH | CH₂ | J71 | 0 | CH |
| 199 | —OCH₂O— | A1 | COOH | CH₂CH₂ | J1 | 0 | CH |
| 200 | —OCH₂O— | A1 | COOH | CH₂ | J4 | 0 | CH |

TABLE 9

| | | | | | | | | |
|---|---|---|---|---|---|---|---|---|
| Com | | | | | | | | |
| pound | | | | | | | | |
| No. | R¹ | R² | SCH₂-A | E | G | J | m | X |

| | | | | | | | |
|---|---|---|---|---|---|---|---|
| 201 | —OCH₂O— | A1 | COOH | CH₂ | J10 | 0 | CH |
| 202 | —OCH₂O— | A1 | COOH | CH₂ | J18 | 0 | CH |
| 203 | —OCH₂O— | A1 | COOH | CH₂ | J21 | 0 | CH |
| 204 | —OCH₂O— | A1 | COOH | CH₂ | J28 | 0 | CH |
| 205 | —OCH₂O— | A1 | COOH | CH₂ | J35 | 0 | CH |
| 206 | —OCH₂O— | A1 | COOH | CH₂ | J37 | 0 | CH |
| 207 | —OCH₂O— | A1 | COOH | CH₂ | J39 | 0 | CH |
| 208 | —OCH₂O— | A1 | COOH | CH₂ | J43 | 0 | CH |
| 209 | —OCH₂O— | A1 | COOH | CH₂ | J46 | 0 | CH |
| 210 | —OCH₂O— | A1 | COOH | CH₂ | J50 | 0 | CH |
| 211 | —OCH₂O— | A1 | COOH | CH₂ | J54 | 0 | CH |
| 212 | —OCH₂O— | A1 | COOH | CH₂ | J63 | 0 | CH |
| 213 | —OCH₂O— | A1 | COOH | CH₂ | J64 | 0 | CH |
| 214 | —OCH₂O— | A1 | COOH | CH₂ | J65 | 0 | CH |
| 215 | —OCH₂O— | A1 | COOH | CH₂ | J66 | 0 | CH |
| 216 | —OCH₂O— | A1 | COOH | CH₂ | J67 | 0 | CH |
| 217 | —OCH₂O— | A1 | COOH | CH₂ | J71 | 0 | CH |
| 218 | —OCH₂CH₂0— | A1 | COOH | CH₂CH₂ | J1 | 0 | CH |
| 219 | —OCH₂CH₂0— | A1 | COOH | CH₂ | J4 | 0 | CH |
| 220 | —OCH₂CH₂0— | A1 | COOH | CH₂ | J10 | 0 | CH |
| 221 | —OCH₂CH₂0— | A1 | COOH | CH₂ | J18 | 0 | CH |
| 222 | —OCH₂CH₂0— | A1 | COOH | CH₂ | J35 | 0 | CH |
| 223 | —OCH₂CH₂0— | A1 | COOH | CH₂ | J37 | 0 | CH |
| 224 | —OCH₂CH₂0— | A1 | COOH | CH₂ | J39 | 0 | CH |
| 225 | —OCH₂CH₂0— | A1 | COOH | CH₂ | J50 | 0 | CH |

TABLE 10

| Com- | | | | | | | | |
|---|---|---|---|---|---|---|---|---|
| pound | | | | | | | | |
| No. | R¹ | R² | SCH₂-A | E | G | J | m | X |
| 226 | —OCH₂CH₂0— | | A1 | COOH | CH₂ | J63 | 0 | CH |
| 227 | —OCH₂CH₂0— | | A1 | COOH | CH₂ | J64 | 0 | CH |
| 228 | —OCH₂CH₂0— | | A1 | COOH | CH₂ | J65 | 0 | CH |
| 229 | —OCH₂CH₂0— | | A1 | COOH | CH₂ | J67 | 0 | CH |
| 230 | —OCH₂CH₂0— | | A1 | COOH | CH₂ | J71 | 0 | CH |
| 231 | OMe | OMe | A1 | COOH | CH₂CH₂ | J1 | 0 | CH |
| 232 | OMe | OMe | A1 | COOH | CH₂ | J4 | 0 | CH |
| 233 | OMe | OMe | A1 | COOH | CH₂ | J10 | 0 | CH |
| 234 | OMe | OMe | A1 | COOH | CH₂ | J18 | 0 | CH |
| 235 | OMe | OMe | A1 | COOH | CH₂ | J35 | 0 | CH |
| 236 | OMe | OMe | A1 | COOH | CH₂ | J37 | 0 | CH |
| 237 | OMe | OMe | A1 | COOH | CH₂ | J39 | 0 | CH |
| 238 | OMe | OMe | A1 | COOH | CH₂ | J50 | 0 | CH |
| 239 | OMe | OMe | A1 | COOH | CH₂ | J63 | 0 | CH |
| 240 | OMe | OMe | A1 | COOH | CH₂ | J64 | 0 | CH |
| 241 | OMe | OMe | A1 | COOH | CH₂ | J65 | 0 | CH |
| 242 | OMe | OMe | A1 | COOH | CH₂ | J67 | 0 | CH |
| 243 | OMe | OMe | A1 | COOH | CH₂ | J71 | 0 | CH |
| 244 | F | F | A1 | COOH | CH₂ | J35 | 0 | CH |
| 245 | F | F | A1 | COOH | CH₂ | J37 | 0 | CH |
| 246 | F | F | A1 | COOH | CH₂ | J39 | 0 | CH |
| 247 | F | F | A1 | COOH | CH₂ | J50 | 0 | CH |
| 248 | F | F | A1 | COOH | CH₂ | J63 | 0 | CH |
| 249 | F | F | A1 | COOH | CH₂ | J64 | 0 | CH |
| 250 | F | F | A1 | COOH | CH₂ | J65 | 0 | CH |

TABLE 11

| Compound No. | R¹ | R² | SCH₂-A | E | G | J | m | X |
|---|---|---|---|---|---|---|---|---|
| 251 | F | F | A1 | COOH | CH₂ | J67 | 0 | CH |
| 252 | H | H | A1 | COOH | CH₂ | J35 | 0 | N |
| 253 | H | H | A1 | COOH | CH₂ | J37 | 0 | N |
| 254 | H | H | A1 | COOH | CH₂ | J39 | 0 | N |
| 255 | H | H | A1 | COOH | CH₂ | J50 | 0 | N |
| 256 | H | H | A1 | COOH | CH₂ | J63 | 0 | N |
| 257 | H | H | A1 | COOH | CH₂ | J64 | 0 | N |
| 258 | H | H | A1 | COOH | CH₂ | J65 | 0 | N |
| 259 | H | H | A1 | COOH | CH₂ | J67 | 0 | N |
| 260 | Me | H | A1 | COOH | CH₂ | J35 | 0 | CH |
| 261 | Me | H | A1 | COOH | CH₂ | J37 | 0 | CH |
| 262 | Me | H | A1 | COOH | CH₂ | J39 | 0 | CH |
| 263 | Me | H | A1 | COOH | CH₂ | J50 | 0 | CH |
| 264 | Me | H | A1 | COOH | CH₂ | J63 | 0 | CH |
| 265 | Me | H | A1 | COOH | CH₂ | J64 | 0 | CH |
| 266 | Me | H | A1 | COOH | CH₂ | J65 | 0 | CH |
| 267 | Me | H | A1 | COOH | CH₂ | J67 | 0 | CH |
| 268 | OMe | H | A1 | COOH | CH₂ | J35 | 0 | CH |
| 269 | OMe | H | A1 | COOH | CH₂ | J37 | 0 | CH |
| 270 | OMe | H | A1 | COOH | CH₂ | J39 | 0 | CH |
| 271 | OMe | H | A1 | COOH | CH₂ | J50 | 0 | CH |
| 272 | OMe | H | A1 | COOH | CH₂ | J63 | 0 | CH |
| 273 | OMe | H | A1 | COOH | CH₂ | J64 | 0 | CH |
| 274 | OMe | H | A1 | COOH | CH₂ | J65 | 0 | CH |
| 275 | OMe | H | A1 | COOH | CH₂ | J67 | 0 | CH |

TABLE 12

| Compound No. | R¹ | R² | SCH₂-A | E | G | J | m | X |
|---|---|---|---|---|---|---|---|---|
| 276 | OEt | H | A1 | COOH | CH₂ | J63 | 0 | CH |
| 277 | OEt | H | A1 | COOH | CH₂ | J64 | 0 | CH |
| 278 | OEt | H | A1 | COOH | CH₂ | J65 | 0 | CH |
| 279 | CF3 | H | A1 | COOH | CH₂ | J63 | 0 | CH |
| 280 | CF3 | H | A1 | COOH | CH₂ | J64 | 0 | CH |
| 281 | CF3 | H | A1 | COOH | CH₂ | J65 | 0 | CH |
| 282 | CN | H | A1 | COOH | CH₂ | J63 | 0 | CH |
| 283 | CN | H | A1 | COOH | CH₂ | J64 | 0 | CH |
| 284 | CN | H | A1 | COOH | CH₂ | J65 | 0 | CH |
| 285 | Cl | H | A1 | COOH | CH₂ | J63 | 0 | N |
| 286 | Cl | H | A1 | COOH | CH₂ | J64 | 0 | N |
| 287 | Cl | H | A1 | COOH | CH₂ | J65 | 0 | N |
| 288 | Me | Me | A2 | COOH | CH₂ | J35 | 0 | CH |
| 289 | Me | Me | A2 | COOH | CH₂ | J37 | 0 | CH |
| 290 | Me | Me | A2 | COOH | CH₂ | J39 | 0 | CH |
| 291 | Me | Me | A2 | COOH | CH₂ | J63 | 0 | CH |
| 292 | Me | Me | A2 | COOH | CH₂ | J64 | 0 | CH |
| 293 | Me | Me | A2 | COOH | CH₂ | J65 | 0 | CH |
| 294 | Me | Me | A2 | COOH | CH₂CH₂ | J1 | 0 | CH |
| 295 | Me | Me | A3 | COOH | CH₂ | J1 | 0 | CH |
| 296 | Me | Me | A3 | COOH | CH₂ | J35 | 0 | CH |
| 297 | Me | Me | A3 | COOH | CH₂ | J37 | 0 | CH |
| 298 | Me | Me | A3 | COOH | CH₂ | J39 | 0 | CH |
| 299 | Me | Me | A3 | COOH | CH₂ | J50 | 0 | CH |
| 300 | Me | Me | A3 | COOH | CH₂ | J63 | 0 | CH |

TABLE 13

| Compound No. | R¹ | R² | SCH₂-A | E | G | J | m | X |
|---|---|---|---|---|---|---|---|---|
| 301 | Me | Me | A3 | COOH | CH₂ | J64 | 0 | CH |
| 302 | Me | Me | A3 | COOH | CH₂ | J65 | 0 | CH |
| 303 | Me | Me | A3 | COOH | CH₂ | J67 | 0 | CH |
| 304 | Me | Me | A3 | COOH | CH₂CH₂ | J1 | 0 | CH |
| 305 | Me | Me | A3 | COOH | CH₂CH₂ | J63 | 0 | CH |
| 306 | Me | Me | A4 | COOH | CH₂ | J1 | 0 | CH |
| 307 | Me | Me | A4 | COOH | CH₂ | J35 | 0 | CH |
| 308 | Me | Me | A4 | COOH | CH₂ | J37 | 0 | CH |
| 309 | Me | Me | A4 | COOH | CH₂ | J39 | 0 | CH |
| 310 | Me | Me | A4 | COOH | CH₂ | J50 | 0 | CH |
| 311 | Me | Me | A4 | COOH | CH₂ | J63 | 0 | CH |
| 312 | Me | Me | A4 | COOH | CH₂ | J64 | 0 | CH |
| 313 | Me | Me | A4 | COOH | CH₂ | J65 | 0 | CH |
| 314 | Me | Me | A4 | COOH | CH₂ | J67 | 0 | CH |
| 315 | Me | Me | A4 | COOH | CH₂CH₂ | J1 | 0 | CH |
| 316 | Me | Me | A4 | COOH | CH₂CH₂ | J63 | 0 | CH |
| 317 | H | H | A4 | COOH | CH₂ | J37 | 0 | CH |
| 318 | H | H | A4 | COOH | CH₂ | J39 | 0 | CH |
| 319 | H | H | A4 | COOH | CH₂ | J63 | 0 | CH |
| 320 | H | H | A4 | COOH | CH₂ | J64 | 0 | CH |
| 321 | H | H | A4 | COOH | CH₂ | J65 | 0 | CH |
| 322 | Cl | Cl | A4 | COOH | CH₂ | J37 | 0 | CH |
| 323 | Cl | Cl | A4 | COOH | CH₂ | J39 | 0 | CH |
| 324 | Cl | Cl | A4 | COOH | CH₂ | J63 | 0 | CH |
| 325 | Cl | Cl | A4 | COOH | CH₂ | J64 | 0 | CH |

TABLE 14

| Compound No. | R¹ | R² | SCH₂-A | E | G | J | m | X |
|---|---|---|---|---|---|---|---|---|
| 326 | Cl | Cl | A4 | COOH | CH₂ | J65 | 0 | CH |
| 327 | H | H | A4 | COOH | CH₂ | J37 | 0 | N |
| 328 | H | H | A4 | COOH | CH₂ | J39 | 0 | N |
| 329 | H | H | A4 | COOH | CH₂ | J63 | 0 | N |
| 330 | H | H | A4 | COOH | CH₂ | J64 | 0 | N |
| 331 | H | H | A4 | COOH | CH₂ | J65 | 0 | N |
| 332 | Me | Me | A5 | COOH | CH₂ | J1 | 0 | CH |
| 333 | Me | Me | A5 | COOH | CH₂CH₂ | J1 | 0 | CH |
| 334 | Me | Me | A6 | COOH | CH₂ | J1 | 0 | CH |
| 335 | Me | Me | A6 | COOH | CH₂CH₂ | J1 | 0 | CH |
| 336 | Me | Me | A7 | COOH | CH₂ | J1 | 0 | CH |
| 337 | Me | Me | A7 | COOH | CH₂CH₂ | J1 | 0 | CH |
| 338 | Me | Me | A8 | COOH | CH₂ | J1 | 0 | CH |
| 339 | Me | Me | A8 | COOH | CH₂CH₂ | J1 | 0 | CH |
| 340 | Me | Me | A9 | COOH | CH₂ | J1 | 0 | CH |
| 341 | Me | Me | A9 | COOH | CH₂CH₂ | J1 | 0 | CH |
| 342 | Me | Me | A10 | COOH | CH₂ | J1 | 0 | CH |
| 343 | Me | Me | A10 | COOH | CH₂CH₂ | J1 | 0 | CH |
| 344 | Me | Me | A11 | COOH | CH₂ | J37 | 0 | CH |
| 345 | Me | Me | A11 | COOH | CH₂ | J39 | 0 | CH |
| 346 | Me | Me | A11 | COOH | CH₂ | J50 | 0 | CH |
| 347 | Me | Me | A11 | COOH | CH₂ | J63 | 0 | CH |
| 348 | Me | Me | A11 | COOH | CH₂ | J64 | 0 | CH |
| 349 | H | H | A11 | COOH | CH₂ | J37 | 0 | CH |
| 350 | H | H | A11 | COOH | CH₂ | J39 | 0 | CH |

TABLE 15

| Compound No. | R¹ | R² | SCH₂-A | E | G | J | m | X |
|---|---|---|---|---|---|---|---|---|
| 351 | H | H | A11 | COOH | CH₂ | J50 | 0 | CH |
| 352 | H | H | A11 | COOH | CH₂ | J63 | 0 | CH |
| 353 | H | H | A11 | COOH | CH₂ | J64 | 0 | CH |
| 354 | H | H | A11 | COOH | CH₂ | J65 | 0 | CH |
| 355 | Cl | Cl | A11 | COOH | CH₂ | J37 | 0 | CH |
| 356 | Cl | Cl | A11 | COOH | CH₂ | J39 | 0 | CH |
| 357 | Cl | Cl | A11 | COOH | CH₂ | J50 | 0 | CH |
| 358 | Cl | Cl | A11 | COOH | CH₂ | J63 | 0 | CH |
| 359 | Cl | Cl | A11 | COOH | CH₂ | J64 | 0 | CH |
| 360 | Cl | Cl | A11 | COOH | CH₂ | J65 | 0 | CH |
| 361 | H | H | A11 | COOH | CH₂ | J37 | 0 | N |
| 362 | H | H | A11 | COOH | CH₂ | J39 | 0 | N |
| 363 | H | H | A11 | COOH | CH₂ | J50 | 0 | N |
| 364 | H | H | A11 | COOH | CH₂ | J63 | 0 | N |
| 365 | H | H | A11 | COOH | CH₂ | J64 | 0 | N |
| 366 | H | H | A11 | COOH | CH₂ | J65 | 0 | N |
| 367 | Me | Me | A12 | COOH | CH₂ | J1 | 0 | CH |
| 368 | Me | Me | A12 | COOH | CH₂CH₂ | J1 | 0 | CH |
| 369 | Me | Me | A13 | COOH | CH₂ | J1 | 0 | CH |
| 370 | Me | Me | A13 | COOH | CH₂CH₂ | J1 | 0 | CH |
| 371 | Me | Me | A14 | COOH | CH₂ | J1 | 0 | CH |
| 372 | Me | Me | A14 | COOH | CH₂CH₂ | J1 | 0 | CH |
| 373 | Me | Me | A15 | COOH | CH₂ | J1 | 0 | CH |
| 374 | Me | Me | A15 | COOH | CH₂CH₂ | J1 | 0 | CH |
| 375 | Me | Me | A16 | COOH | CH₂ | J1 | 0 | CH |

TABLE 16

| Compound No. | R¹ | R² | SCH₂-A | E | G | J | m | X |
|---|---|---|---|---|---|---|---|---|
| 376 | Me | Me | A16 | COOH | CH₂CH₂ | J1 | 0 | CH |
| 377 | Me | Me | A16 | COOH | CH₂ | J37 | 0 | CH |
| 378 | Me | Me | A16 | COOH | CH₂ | J39 | 0 | CH |
| 379 | Me | Me | A16 | COOH | CH₂ | J50 | 0 | CH |
| 380 | Me | Me | A16 | COOH | CH₂ | J63 | 0 | CH |
| 381 | Me | Me | A16 | COOH | CH₂ | J64 | 0 | CH |
| 382 | Me | Me | A16 | COOH | CH₂ | J65 | 0 | CH |
| 383 | H | H | A16 | COOH | CH₂ | J37 | 0 | CH |
| 384 | H | H | A16 | COOH | CH₂ | J39 | 0 | CH |
| 385 | H | H | A16 | COOH | CH₂ | J50 | 0 | CH |
| 386 | H | H | A16 | COOH | CH₂ | J63 | 0 | CH |
| 387 | H | H | A16 | COOH | CH₂ | J64 | 0 | CH |
| 388 | H | H | A16 | COOH | CH₂ | J65 | 0 | CH |
| 389 | Me | Me | A17 | COOH | CH₂ | J1 | 0 | CH |
| 390 | Me | Me | A17 | COOH | CH₂CH₂ | J1 | 0 | CH |
| 391 | Me | Me | A18 | COOH | CH₂CH₂ | J1 | 0 | CH |
| 392 | Me | Me | A18 | COOH | CH₂ | J37 | 0 | CH |
| 393 | Me | Me | A18 | COOH | CH₂ | J39 | 0 | CH |
| 394 | Me | Me | A18 | COOH | CH₂ | J50 | 0 | CH |
| 395 | Me | Me | A18 | COOH | CH₂ | J63 | 0 | CH |
| 396 | Me | Me | A18 | COOH | CH₂ | J64 | 0 | CH |
| 397 | Me | Me | A18 | COOH | CH₂ | J65 | 0 | CH |
| 398 | H | H | A18 | COOH | CH₂ | J37 | 0 | CH |
| 399 | H | H | A18 | COOH | CH₂ | J39 | 0 | CH |
| 400 | H | H | A18 | COOH | CH₂ | J50 | 0 | CH |

TABLE 17

| Compound No. | R¹ | R² | SCH₂-A | E | G | J | m | X |
|---|---|---|---|---|---|---|---|---|
| 401 | H | H | A18 | COOH | CH₂ | J63 | 0 | CH |
| 402 | H | H | A18 | COOH | CH₂ | J64 | 0 | CH |
| 403 | H | H | A18 | COOH | CH₂ | J65 | 0 | CH |
| 404 | Cl | Cl | A18 | COOH | CH₂ | J37 | 0 | CH |
| 405 | Cl | Cl | A18 | COOH | CH₂ | J63 | 0 | CH |
| 406 | Cl | Cl | A18 | COOH | CH₂ | J64 | 0 | CH |
| 407 | Cl | Cl | A18 | COOH | CH₂ | J65 | 0 | CH |
| 408 | H | H | A18 | COOH | CH₂ | J37 | 0 | N |
| 409 | H | H | A18 | COOH | CH₂ | J39 | 0 | N |
| 410 | H | H | A18 | COOH | CH₂ | J63 | 0 | N |
| 411 | H | H | A18 | COOH | CH₂ | J64 | 0 | N |
| 412 | H | H | A18 | COOH | CH₂ | J65 | 0 | N |
| 413 | Me | H | A18 | COOH | CH₂ | J37 | 0 | CH |
| 414 | Me | H | A18 | COOH | CH₂ | J39 | 0 | CH |
| 415 | Me | H | A18 | COOH | CH₂ | J63 | 0 | CH |
| 416 | Me | H | A18 | COOH | CH₂ | J64 | 0 | CH |
| 417 | Me | H | A18 | COOH | CH₂ | J65 | 0 | CH |
| 418 | OMe | H | A18 | COOH | CH₂ | J37 | 0 | CH |
| 419 | OMe | H | A18 | COOH | CH₂ | J39 | 0 | CH |
| 420 | OMe | H | A18 | COOH | CH₂ | J63 | 0 | CH |
| 421 | OMe | H | A18 | COOH | CH₂ | J64 | 0 | CH |
| 422 | OMe | H | A18 | COOH | CH₂ | J65 | 0 | CH |
| 423 | OEt | H | A18 | COOH | CH₂ | J63 | 0 | CH |
| 424 | OEt | H | A18 | COOH | CH₂ | J64 | 0 | CH |
| 425 | OEt | H | A18 | COOH | CH₂ | J65 | 0 | CH |

TABLE 18

| Compound No. | R¹ | R² | SCH₂-A | E | G | J | m | X |
|---|---|---|---|---|---|---|---|---|
| 426 | CF3 | H | A18 | COOH | CH₂ | J63 | 0 | CH |
| 427 | CF3 | H | A18 | COOH | CH₂ | J64 | 0 | CH |
| 428 | CF3 | H | A18 | COOH | CH₂ | J65 | 0 | CH |
| 429 | CN | H | A18 | COOH | CH₂ | J63 | 0 | CH |
| 430 | CN | H | A18 | COOH | CH₂ | J64 | 0 | CH |
| 431 | CN | H | A18 | COOH | CH₂ | J65 | 0 | CH |
| 432 | F | H | A18 | COOH | CH₂ | J63 | 0 | CH |
| 433 | F | H | A18 | COOH | CH₂ | J64 | 0 | CH |
| 434 | F | H | A18 | COOH | CH₂ | J65 | 0 | CH |
| 435 | Cl | H | A18 | COOH | CH₂ | J63 | 0 | N |
| 436 | Cl | H | A18 | COOH | CH₂ | J64 | 0 | N |
| 437 | Cl | H | A18 | COOH | CH₂ | J65 | 0 | N |
| 438 | H | H | A18 | COOH | CH₂ | J37 | 0 | N |
| 439 | Me | Me | A19 | COOH | CH₂ | J1 | 0 | CH |
| 440 | Me | Me | A19 | COOH | CH₂CH₂ | J1 | 0 | CH |
| 441 | Me | Me | A19 | COOH | CH₂ | J37 | 0 | CH |
| 442 | Me | Me | A19 | COOH | CH₂ | J39 | 0 | CH |
| 443 | Me | Me | A19 | COOH | CH₂ | J50 | 0 | CH |
| 444 | Me | Me | A19 | COOH | CH₂ | J63 | 0 | CH |
| 445 | Me | Me | A19 | COOH | CH₂ | J64 | 0 | CH |
| 446 | Me | Me | A19 | COOH | CH₂ | J65 | 0 | CH |
| 447 | H | H | A19 | COOH | CH₂ | J1 | 0 | CH |
| 448 | H | H | A19 | COOH | CH₂CH₂ | J1 | 0 | CH |
| 449 | H | H | A19 | COOH | CH₂ | J37 | 0 | CH |
| 450 | H | H | A19 | COOH | CH₂ | J39 | 0 | CH |

TABLE 19

| Compound No. | R¹ | R² | SCH₂-A | E | G | J | m | X |
|---|---|---|---|---|---|---|---|---|
| 451 | H | H | A19 | COOH | CH₂ | J50 | 0 | CH |
| 452 | H | H | A19 | COOH | CH₂ | J63 | 0 | CH |
| 453 | H | H | A19 | COOH | CH₂ | J64 | 0 | CH |
| 454 | H | H | A19 | COOH | CH₂ | J65 | 0 | CH |
| 455 | Me | Me | A20 | COOH | CH₂ | J64 | 0 | CH |
| 456 | Me | Me | A20 | COOH | CH₂ | J65 | 0 | CH |
| 457 | Me | Me | A20 | COOH | CH₂ | J67 | 0 | CH |
| 458 | Me | Me | A20 | COOH | CH₂ | J71 | 0 | CH |
| 459 | H | H | A20 | COOH | CH₂ | J64 | 0 | CH |
| 460 | H | H | A20 | COOH | CH₂ | J65 | 0 | CH |
| 461 | H | H | A20 | COOH | CH₂ | J67 | 0 | CH |
| 462 | H | H | A20 | COOH | CH₂ | J71 | 0 | CH |
| 463 | Cl | Cl | A20 | COOH | CH₂ | J64 | 0 | CH |
| 464 | Cl | Cl | A20 | COOH | CH₂ | J65 | 0 | CH |
| 465 | Cl | Cl | A20 | COOH | CH₂ | J67 | 0 | CH |
| 466 | Cl | Cl | A20 | COOH | CH₂ | J71 | 0 | CH |
| 467 | H | H | A20 | COOH | CH₂ | J64 | 0 | N |
| 468 | H | H | A20 | COOH | CH₂ | J65 | 0 | N |
| 469 | H | H | A20 | COOH | CH₂ | J67 | 0 | N |
| 470 | H | H | A20 | COOH | CH₂ | J71 | 0 | N |
| 471 | Me | H | A20 | COOH | CH₂ | J64 | 0 | CH |
| 472 | Me | H | A20 | COOH | CH₂ | J65 | 0 | CH |
| 473 | Me | H | A20 | COOH | CH₂ | J67 | 0 | CH |
| 474 | Me | H | A20 | COOH | CH₂ | J71 | 0 | CH |
| 475 | OMe | H | A20 | COOH | CH₂ | J64 | 0 | CH |

TABLE 20

| Compound No. | R¹ | R² | SCH₂-A | E | G | J | m | X |
|---|---|---|---|---|---|---|---|---|
| 476 | OMe | H | A20 | COOH | CH₂ | J65 | 0 | CH |
| 477 | OMe | H | A20 | COOH | CH₂ | J67 | 0 | CH |
| 478 | OMe | H | A20 | COOH | CH₂ | J71 | 0 | CH |
| 479 | OEt | H | A20 | COOH | CH₂ | J64 | 0 | CH |
| 480 | OEt | H | A20 | COOH | CH₂ | J65 | 0 | CH |
| 481 | OEt | H | A20 | COOH | CH₂ | J67 | 0 | CH |
| 482 | OEt | H | A20 | COOH | CH₂ | J71 | 0 | CH |
| 483 | F | H | A20 | COOH | CH₂ | J64 | 0 | CH |
| 484 | F | H | A20 | COOH | CH₂ | J65 | 0 | CH |
| 485 | F | H | A20 | COOH | CH₂ | J67 | 0 | CH |
| 486 | F | H | A20 | COOH | CH₂ | J71 | 0 | CH |
| 487 | CF3 | H | A20 | COOH | CH₂ | J64 | 0 | CH |
| 488 | CF3 | H | A20 | COOH | CH₂ | J65 | 0 | CH |
| 489 | CF3 | H | A20 | COOH | CH₂ | J67 | 0 | CH |
| 490 | CF3 | H | A20 | COOH | CH₂ | J71 | 0 | CH |
| 491 | CN | H | A20 | COOH | CH₂ | J64 | 0 | CH |
| 492 | CN | H | A20 | COOH | CH₂ | J65 | 0 | CH |
| 493 | CN | H | A20 | COOH | CH₂ | J67 | 0 | CH |
| 494 | CN | H | A20 | COOH | CH₂ | J71 | 0 | CH |
| 495 | Cl | H | A20 | COOH | CH₂ | J64 | 0 | N |
| 496 | Cl | H | A20 | COOH | CH₂ | J65 | 0 | N |
| 497 | Cl | H | A20 | COOH | CH₂ | J67 | 0 | N |
| 498 | Cl | H | A20 | COOH | CH₂ | J71 | 0 | N |
| 499 | H | H | A21 | COOH | CH₂ | J63 | 0 | CH |
| 500 | H | H | A21 | COOH | CH₂ | J65 | 0 | CH |

TABLE 21

| Compound | | | | | | | | |
|---|---|---|---|---|---|---|---|---|
| No. | R¹ | R² | SCH₂-A | E | G | J | m | X |
| 501 | Me | Me | A1 | COOH | CH₂CH₂ | J1 | 0 | CH |
| 502 | Me | Me | A1 | COOH | CH₂CH₂ | J37 | 0 | CH |
| 503 | Me | Me | A1 | COOH | CH₂CH₂ | J39 | 0 | CH |
| 504 | Me | Me | A1 | COOH | CH₂CH₂ | J50 | 0 | CH |
| 505 | Me | Me | A1 | COOH | CH₂CH₂ | J62 | 0 | CH |
| 506 | Me | Me | A1 | COOH | CH₂CH₂ | J63 | 0 | CH |
| 507 | Me | Me | A1 | COOH | CH₂CH₂ | J64 | 0 | CH |
| 508 | Me | Me | A1 | COOH | CH₂CH₂ | J65 | 0 | CH |
| 509 | H | H | A1 | COOH | CH₂CH₂ | J1 | 0 | CH |
| 510 | H | H | A1 | COOH | CH₂CH₂ | J37 | 0 | CH |
| 511 | H | H | A1 | COOH | CH₂CH₂ | J39 | 0 | CH |
| 512 | H | H | A1 | COOH | CH₂CH₂ | J50 | 0 | CH |
| 513 | H | H | A1 | COOH | CH₂CH₂ | J62 | 0 | CH |
| 514 | H | H | A1 | COOH | CH₂CH₂ | J63 | 0 | CH |
| 515 | H | H | A1 | COOH | CH₂CH₂ | J64 | 0 | CH |
| 516 | H | H | A1 | COOH | CH₂CH₂ | J65 | 0 | CH |
| 517 | Me | Me | A4 | COOH | CH₂CH₂ | J37 | 0 | CH |
| 518 | Me | Me | A4 | COOH | CH₂CH₂ | J39 | 0 | CH |
| 519 | Me | Me | A4 | COOH | CH₂CH₂ | J67 | 0 | CH |
| 520 | Me | Me | A4 | COOH | CH₂CH₂ | J64 | 0 | CH |
| 521 | Me | Me | A4 | COOH | CH₂CH₂ | J65 | 0 | CH |
| 522 | H | H | A4 | COOH | CH₂CH₂ | J37 | 0 | CH |
| 523 | H | H | A4 | COOH | CH₂CH₂ | J39 | 0 | CH |
| 524 | H | H | A4 | COOH | CH₂CH₂ | J63 | 0 | CH |
| 525 | H | H | A4 | COOH | CH₂CH₂ | J64 | 0 | CH |

TABLE 22

| Compound | | | | | | | | |
|---|---|---|---|---|---|---|---|---|
| No. | R¹ | R² | SCH₂-A | E | G | J | m | X |
| 526 | H | H | A4 | COOH | CH₂CH₂ | J65 | 0 | CH |
| 527 | H | H | A11 | COOH | CH₂CH₂ | J37 | 0 | CH |
| 528 | H | H | A11 | COOH | CH₂CH₂ | J39 | 0 | CH |
| 529 | H | H | A11 | COOH | CH₂CH₂ | J63 | 0 | CH |
| 530 | H | H | A11 | COOH | CH₂CH₂ | J64 | 0 | CH |
| 531 | H | H | A11 | COOH | CH₂CH₂ | J65 | 0 | CH |
| 532 | H | H | A18 | COOH | CH₂CH₂ | J37 | 0 | CH |
| 533 | H | H | A18 | COOH | CH₂CH₂ | J39 | 0 | CH |
| 534 | H | H | A18 | COOH | CH₂CH₂ | J63 | 0 | CH |
| 535 | H | H | A18 | COOH | CH₂CH₂ | J64 | 0 | CH |
| 536 | H | H | A18 | COOH | CH₂CH₂ | J65 | 0 | CH |
| 537 | Me | Me | A20 | COOH | CH₂CH₂ | J37 | 0 | CH |
| 538 | Me | Me | A20 | COOH | CH₂CH₂ | J39 | 0 | CH |
| 539 | Me | Me | A20 | COOH | CH₂CH₂ | J63 | 0 | CH |
| 540 | Me | Me | A20 | COOH | CH₂CH₂ | J64 | 0 | CH |
| 541 | Me | Me | A20 | COOH | CH₂CH₂ | J65 | 0 | CH |
| 542 | H | H | A20 | COOH | CH₂CH₂ | J37 | 0 | CH |
| 543 | H | H | A20 | COOH | CH₂CH₂ | J39 | 0 | CH |
| 544 | H | H | A20 | COOH | CH₂CH₂ | J63 | 0 | CH |
| 545 | H | H | A20 | COOH | CH₂CH₂ | J64 | 0 | CH |
| 546 | H | H | A20 | COOH | CH₂CH₂ | J65 | 0 | CH |
| 547 | Me | Me | A1 | COOH | CO | J1 | 0 | CH |
| 548 | Me | Me | A1 | COOH | CO | J63 | 0 | CH |
| 549 | H | H | A1 | COOH | CO | J1 | 0 | CH |
| 550 | H | H | A1 | COOH | CO | J63 | 0 | CH |

TABLE 23

| Compound | | | | | | | | |
|---|---|---|---|---|---|---|---|---|
| No. | R¹ | R² | SCH₂-A | E | G | J | m | X |
| 551 | Me | Me | A4 | COOH | CO | J1 | 0 | CH |
| 552 | Me | Me | A4 | COOH | CO | J63 | 0 | CH |
| 553 | H | H | A4 | COOH | CO | J1 | 0 | CH |
| 554 | H | H | A4 | COOH | CO | J63 | 0 | CH |
| 555 | H | H | A11 | COOH | CO | J1 | 0 | CH |
| 556 | H | H | A11 | COOH | CO | J63 | 0 | CH |
| 557 | H | H | A18 | COOH | CO | J1 | 0 | CH |
| 558 | H | H | A18 | COOH | CO | J63 | 0 | CH |
| 559 | H | H | A20 | COOH | CO | J1 | 0 | CH |
| 560 | H | H | A20 | COOH | CO | J63 | 0 | CH |
| 561 | Me | Me | A1 | COOH | SO₂ | J1 | 0 | CH |
| 562 | Me | Me | A1 | COOH | SO₂ | J63 | 0 | CH |
| 563 | H | H | A1 | COOH | SO₂ | J1 | 0 | CH |
| 564 | H | H | A1 | COOH | SO₂ | J63 | 0 | CH |
| 565 | H | H | A4 | COOH | SO₂ | J1 | 0 | CH |
| 566 | H | H | A4 | COOH | SO₂ | J63 | 0 | CH |
| 567 | H | H | A11 | COOH | SO₂ | J1 | 0 | CH |
| 568 | H | H | A11 | COOH | SO₂ | J63 | 0 | CH |
| 569 | H | H | A18 | COOH | SO₂ | J1 | 0 | CH |
| 570 | H | H | A18 | COOH | SO₂ | J63 | 0 | CH |
| 571 | H | H | A20 | COOH | SO₂ | J1 | 0 | CH |
| 572 | H | H | A20 | COOH | SO₂ | J63 | 0 | CH |
| 573 | H | H | A1 | COOH | CH₂CO | J1 | 0 | CH |
| 574 | H | H | A1 | COOH | CH₂CO | J2 | 0 | CH |
| 575 | H | H | A1 | COOH | CH₂CO | J3 | 0 | CH |

TABLE 24

| Compound | | | | | | | | |
|---|---|---|---|---|---|---|---|---|
| No. | R¹ | R² | SCH₂-A | E | G | J | m | X |
| 576 | H | H | A1 | COOH | CH₂CO | J4 | 0 | CH |
| 577 | H | H | A1 | COOH | CH₂CO | J5 | 0 | CH |
| 578 | H | H | A1 | COOH | CH₂CO | J6 | 0 | CH |
| 579 | H | H | A1 | COOH | CH₂CO | J7 | 0 | CH |
| 580 | H | H | A1 | COOH | CH₂CO | J8 | 0 | CH |
| 581 | H | H | A1 | COOH | CH₂CO | J9 | 0 | CH |
| 582 | H | H | A1 | COOH | CH₂CO | J10 | 0 | CH |
| 583 | H | H | A1 | COOH | CH₂CO | J11 | 0 | CH |
| 584 | H | H | A1 | COOH | CH₂CO | J12 | 0 | CH |
| 585 | H | H | A1 | COOH | CH₂CO | J13 | 0 | CH |
| 586 | H | H | A1 | COOH | CH₂CO | J17 | 0 | CH |
| 587 | H | H | A1 | COOH | CH₂CO | J18 | 0 | CH |
| 588 | H | H | A1 | COOH | CH₂CO | J19 | 0 | CH |
| 589 | H | H | A1 | COOH | CH₂CO | J23 | 0 | CH |
| 590 | H | H | A1 | COOH | CH₂CO | J24 | 0 | CH |
| 591 | H | H | A1 | COOH | CH₂CO | J25 | 0 | CH |
| 592 | H | H | A1 | COOH | CH₂CO | J36 | 0 | CH |
| 593 | H | H | A1 | COOH | CH₂CO | J47 | 0 | CH |
| 594 | H | H | A1 | COOH | CH₂CO | J57 | 0 | CH |
| 595 | H | H | A1 | COOH | CH₂CO | J62 | 0 | CH |
| 596 | Me | Me | A1 | COOH | CH₂CO | J1 | 0 | CH |
| 597 | Me | Me | A1 | COOH | CH₂CO | J2 | 0 | CH |
| 598 | Me | Me | A1 | COOH | CH₂CO | J3 | 0 | CH |
| 599 | Me | Me | A1 | COOH | CH₂CO | J4 | 0 | CH |
| 600 | Me | Me | A1 | COOH | CH₂CO | J5 | 0 | CH |

TABLE 25

| Compound | | | | | | | | |
|---|---|---|---|---|---|---|---|---|
| No. | R¹ | R² | SCH₂-A | E | G | J | m | X |
| 601 | Me | Me | A1 | COOH | CH₂CO | J6 | 0 | CH |
| 602 | Me | Me | A1 | COOH | CH₂CO | J7 | 0 | CH |
| 603 | Me | Me | A1 | COOH | CH₂CO | J8 | 0 | CH |
| 604 | Me | Me | A1 | COOH | CH₂CO | J9 | 0 | CH |
| 605 | Me | Me | A1 | COOH | CH₂CO | J10 | 0 | CH |
| 606 | Me | Me | A1 | COOH | CH₂CO | J11 | 0 | CH |
| 607 | Me | Me | A1 | COOH | CH₂CO | J12 | 0 | CH |
| 608 | Me | Me | A1 | COOH | CH₂CO | J13 | 0 | CH |
| 609 | Me | Me | A1 | COOH | CH₂CO | J17 | 0 | CH |
| 610 | Me | Me | A1 | COOH | CH₂CO | J18 | 0 | CH |
| 611 | Me | Me | A1 | COOH | CH₂CO | J19 | 0 | CH |
| 612 | Me | Me | A1 | COOH | CH₂CO | J23 | 0 | CH |
| 613 | Me | Me | A1 | COOH | CH₂CO | J24 | 0 | CH |
| 614 | Me | Me | A1 | COOH | CH₂CO | J25 | 0 | CH |
| 615 | Me | Me | A1 | COOH | CH₂CO | J36 | 0 | CH |
| 616 | Me | Me | A1 | COOH | CH₂CO | J47 | 0 | CH |
| 617 | Me | Me | A1 | COOH | CH₂CO | J57 | 0 | CH |
| 618 | Me | Me | A1 | COOH | CH₂CO | J62 | 0 | CH |
| 619 | H | H | A1 | COOH | CH₂CONH | J1 | 0 | CH |
| 620 | H | H | A1 | COOH | CH₂CONH | J2 | 0 | CH |
| 621 | H | H | A1 | COOH | CH₂CONH | J3 | 0 | CH |
| 622 | H | H | A1 | COOH | CH₂CONH | J4 | 0 | CH |
| 623 | H | H | A1 | COOH | CH₂CONH | J5 | 0 | CH |
| 624 | H | H | A1 | COOH | CH₂CONH | J6 | 0 | CH |
| 625 | H | H | A1 | COOH | CH₂CONH | J7 | 0 | CH |

TABLE 26

| Compound | | | | | | | | |
|---|---|---|---|---|---|---|---|---|
| No. | R¹ | R² | SCH₂-A | E | G | J | m | X |
| 626 | H | H | A1 | COOH | CH₂CONH | J8 | 0 | CH |
| 627 | H | H | A1 | COOH | CH₂CONH | J9 | 0 | CH |
| 628 | H | H | A1 | COOH | CH₂CONH | J10 | 0 | CH |
| 629 | H | H | A1 | COOH | CH₂CONH | J11 | 0 | CH |
| 630 | H | H | A1 | COOH | CH₂CONH | J12 | 0 | CH |
| 631 | H | H | A1 | COOH | CH₂CONH | J13 | 0 | CH |
| 632 | H | H | A1 | COOH | CH₂CONH | J14 | 0 | CH |
| 633 | H | H | A1 | COOH | CH₂CONH | J15 | 0 | CH |
| 634 | H | H | A1 | COOH | CH₂CONH | J16 | 0 | CH |
| 635 | H | H | A1 | COOH | CH₂CONH | J17 | 0 | CH |
| 636 | H | H | A1 | COOH | CH₂CONH | J18 | 0 | CH |
| 637 | H | H | A1 | COOH | CH₂CONH | J19 | 0 | CH |
| 638 | H | H | A1 | COOH | CH₂CONH | J20 | 0 | CH |
| 639 | H | H | A1 | COOH | CH₂CONH | J21 | 0 | CH |
| 640 | H | H | A1 | COOH | CH₂CONH | J22 | 0 | CH |
| 641 | H | H | A1 | COOH | CH₂CONH | J23 | 0 | CH |
| 642 | H | H | A1 | COOH | CH₂CONH | J24 | 0 | CH |
| 643 | H | H | A1 | COOH | CH₂CONH | J25 | 0 | CH |
| 644 | H | H | A1 | COOH | CH₂CONH | J26 | 0 | CH |
| 645 | H | H | A1 | COOH | CH₂CONH | J27 | 0 | CH |
| 646 | H | H | A1 | COOH | CH₂CONH | J28 | 0 | CH |
| 647 | H | H | A1 | COOH | CH₂CONH | J29 | 0 | CH |
| 648 | H | H | A1 | COOH | CH₂CONH | J30 | 0 | CH |
| 649 | H | H | A1 | COOH | CH₂CONH | J31 | 0 | CH |
| 650 | H | H | A1 | COOH | CH₂CONH | J32 | 0 | CH |

TABLE 27

| Compound | | | | | | | | |
|---|---|---|---|---|---|---|---|---|
| No. | R¹ | R² | SCH₂-A | E | G | J | m | X |
| 651 | H | H | A1 | COOH | CH₂CONH | J33 | 0 | CH |
| 652 | H | H | A1 | COOH | CH₂CONH | J34 | 0 | CH |
| 653 | H | H | A1 | COOH | CH₂CONH | J35 | 0 | CH |
| 654 | H | H | A1 | COOH | CH₂CONH | J37 | 0 | CH |
| 655 | H | H | A1 | COOH | CH₂CONH | J39 | 0 | CH |
| 656 | H | H | A1 | COOH | CH₂CONH | J62 | 0 | CH |
| 657 | H | H | A1 | COOH | CH₂CONH | J63 | 0 | CH |
| 658 | Me | Me | A1 | COOH | CH₂CONH | J1 | 0 | CH |
| 659 | Me | Me | A1 | COOH | CH₂CONH | J2 | 0 | CH |
| 660 | Me | Me | A1 | COOH | CH₂CONH | J3 | 0 | CH |
| 661 | Me | Me | A1 | COOH | CH₂CONH | J4 | 0 | CH |
| 662 | Me | Me | A1 | COOH | CH₂CONH | J5 | 0 | CH |
| 663 | Me | Me | A1 | COOH | CH₂CONH | J6 | 0 | CH |
| 664 | Me | Me | A1 | COOH | CH₂CONH | J7 | 0 | CH |
| 665 | Me | Me | A1 | COOH | CH₂CONH | J8 | 0 | CH |
| 666 | Me | Me | A1 | COOH | CH₂CONH | J9 | 0 | CH |
| 667 | Me | Me | A1 | COOH | CH₂CONH | J10 | 0 | CH |
| 668 | Me | Me | A1 | COOH | CH₂CONH | J11 | 0 | CH |
| 669 | Me | Me | A1 | COOH | CH₂CONH | J12 | 0 | CH |
| 670 | Me | Me | A1 | COOH | CH₂CONH | J13 | 0 | CH |
| 671 | Me | Me | A1 | COOH | CH₂CONH | J14 | 0 | CH |
| 672 | Me | Me | A1 | COOH | CH₂CONH | J15 | 0 | CH |
| 673 | Me | Me | A1 | COOH | CH₂CONH | J16 | 0 | CH |
| 674 | Me | Me | A1 | COOH | CH₂CONH | J17 | 0 | CH |
| 675 | Me | Me | A1 | COOH | CH₂CONH | J18 | 0 | CH |

TABLE 28

| Compound | | | | | | | | |
|---|---|---|---|---|---|---|---|---|
| No. | R¹ | R² | SCH₂-A | E | G | J | m | X |
| 676 | Me | Me | A1 | COOH | CH₂CONH | J19 | 0 | CH |
| 677 | Me | Me | A1 | COOH | CH₂CONH | J20 | 0 | CH |
| 678 | Me | Me | A1 | COOH | CH₂CONH | J21 | 0 | CH |
| 679 | Me | Me | A1 | COOH | CH₂CONH | J22 | 0 | CH |
| 680 | Me | Me | A1 | COOH | CH₂CONH | J23 | 0 | CH |
| 681 | Me | Me | A1 | COOH | CH₂CONH | J24 | 0 | CH |
| 682 | Me | Me | A1 | COOH | CH₂CONH | J25 | 0 | CH |
| 683 | Me | Me | A1 | COOH | CH₂CONH | J26 | 0 | CH |
| 684 | Me | Me | A1 | COOH | CH₂CONH | J27 | 0 | CH |
| 685 | Me | Me | A1 | COOH | CH₂CONH | J28 | 0 | CH |
| 686 | Me | Me | A1 | COOH | CH₂CONH | J29 | 0 | CH |
| 687 | Me | Me | A1 | COOH | CH₂CONH | J30 | 0 | CH |
| 688 | Me | Me | A1 | COOH | CH₂CONH | J31 | 0 | CH |
| 689 | Me | Me | A1 | COOH | CH₂CONH | J32 | 0 | CH |
| 690 | Me | Me | A1 | COOH | CH₂CONH | J33 | 0 | CH |
| 691 | Me | Me | A1 | COOH | CH₂CONH | J34 | 0 | CH |
| 692 | Me | Me | A1 | COOH | CH₂CONH | J35 | 0 | CH |
| 693 | Me | Me | A1 | COOH | CH₂CONH | J37 | 0 | CH |
| 694 | Me | Me | A1 | COOH | CH₂CONH | J39 | 0 | CH |
| 695 | Me | Me | A1 | COOH | CH₂CONH | J62 | 0 | CH |
| 696 | Me | Me | A1 | COOH | CH₂CONH | J63 | 0 | CH |
| 697 | H | H | A1 | COOH | CH₂CH₂O | J1 | 0 | CH |
| 698 | H | H | A1 | COOH | CH₂CH₂O | J2 | 0 | CH |
| 699 | H | H | A1 | COOH | CH₂CH₂O | J3 | 0 | CH |
| 700 | H | H | A1 | COOH | CH₂CH₂O | J4 | 0 | CH |

TABLE 29

| Compound | | | | | | | | |
|---|---|---|---|---|---|---|---|---|
| No. | R¹ | R² | SCH₂-A | E | G | J | m | X |
| 701 | H | H | A1 | COOH | CH₂CH₂O | J5 | 0 | CH |
| 702 | H | H | A1 | COOH | CH₂CH₂O | J6 | 0 | CH |
| 703 | H | H | A1 | COOH | CH₂CH₂O | J7 | 0 | CH |
| 704 | H | H | A1 | COOH | CH₂CH₂O | J8 | 0 | CH |
| 705 | H | H | A1 | COOH | CH₂CH₂O | J9 | 0 | CH |
| 706 | H | H | A1 | COOH | CH₂CH₂O | J10 | 0 | CH |
| 707 | H | H | A1 | COOH | CH₂CH₂O | J11 | 0 | CH |
| 708 | H | H | A1 | COOH | CH₂CH₂O | J12 | 0 | CH |
| 709 | H | H | A1 | COOH | CH₂CH₂O | J13 | 0 | CH |
| 710 | H | H | A1 | COOH | CH₂CH₂O | J14 | 0 | CH |
| 711 | H | H | A1 | COOH | CH₂CH₂O | J15 | 0 | CH |
| 712 | H | H | A1 | COOH | CH₂CH₂O | J16 | 0 | CH |
| 713 | H | H | A1 | COOH | CH₂CH₂O | J17 | 0 | CH |
| 714 | H | H | A1 | COOH | CH₂CH₂O | J18 | 0 | CH |
| 715 | H | H | A1 | COOH | CH₂CH₂O | J19 | 0 | CH |
| 716 | H | H | A1 | COOH | CH₂CH₂O | J20 | 0 | CH |
| 717 | H | H | A1 | COOH | CH₂CH₂O | J21 | 0 | CH |
| 718 | H | H | A1 | COOH | CH₂CH₂O | J22 | 0 | CH |
| 719 | H | H | A1 | COOH | CH₂CH₂O | J23 | 0 | CH |
| 720 | H | H | A1 | COOH | CH₂CH₂O | J24 | 0 | CH |
| 721 | H | H | A1 | COOH | CH₂CH₂O | J25 | 0 | CH |
| 722 | H | H | A1 | COOH | CH₂CH₂O | J26 | 0 | CH |
| 723 | H | H | A1 | COOH | CH₂CH₂O | J27 | 0 | CH |
| 724 | H | H | A1 | COOH | CH₂CH₂O | J28 | 0 | CH |
| 725 | H | H | A1 | COOH | CH₂CH₂O | J29 | 0 | CH |

TABLE 30

| Compound | | | | | | | | |
|---|---|---|---|---|---|---|---|---|
| No. | R¹ | R² | SCH₂-A | E | G | J | m | X |
| 726 | H | H | A1 | COOH | CH₂CH₂O | J30 | 0 | CH |
| 727 | H | H | A1 | COOH | CH₂CH₂O | J31 | 0 | CH |
| 728 | H | H | A1 | COOH | CH₂CH₂O | J32 | 0 | CH |
| 729 | H | H | A1 | COOH | CH₂CH₂O | J33 | 0 | CH |
| 730 | H | H | A1 | COOH | CH₂CH₂O | J34 | 0 | CH |
| 731 | H | H | A1 | COOH | CH₂CH₂O | J35 | 0 | CH |
| 732 | H | H | A1 | COOH | CH₂CH₂O | J37 | 0 | CH |
| 733 | H | H | A1 | COOH | CH₂CH₂O | J39 | 0 | CH |
| 734 | H | H | A1 | COOH | CH₂CH₂O | J62 | 0 | CH |
| 735 | H | H | A1 | COOH | CH₂CH₂O | J63 | 0 | CH |
| 736 | Me | Me | A1 | COOH | CH₂CH₂O | J1 | 0 | CH |
| 737 | Me | Me | A1 | COOH | CH₂CH₂O | J2 | 0 | CH |
| 738 | Me | Me | A1 | COOH | CH₂CH₂O | J3 | 0 | CH |
| 739 | Me | Me | A1 | COOH | CH₂CH₂O | J4 | 0 | CH |
| 740 | Me | Me | A1 | COOH | CH₂CH₂O | J5 | 0 | CH |
| 741 | Me | Me | A1 | COOH | CH₂CH₂O | J6 | 0 | CH |
| 742 | Me | Me | A1 | COOH | CH₂CH₂O | J7 | 0 | CH |
| 743 | Me | Me | A1 | COOH | CH₂CH₂O | J8 | 0 | CH |
| 744 | Me | Me | A1 | COOH | CH₂CH₂O | J9 | 0 | CH |
| 745 | Me | Me | A1 | COOH | CH₂CH₂O | J10 | 0 | CH |
| 746 | Me | Me | A1 | COOH | CH₂CH₂O | J11 | 0 | CH |
| 747 | Me | Me | A1 | COOH | CH₂CH₂O | J12 | 0 | CH |
| 748 | Me | Me | A1 | COOH | CH₂CH₂O | J13 | 0 | CH |
| 749 | Me | Me | A1 | COOH | CH₂CH₂O | J14 | 0 | CH |
| 750 | Me | Me | A1 | COOH | CH₂CH₂O | J15 | 0 | CH |

TABLE 31

| Compound | | | | | | | | |
|---|---|---|---|---|---|---|---|---|
| No. | R¹ | R² | SCH₂-A | E | G | J | m | X |
| 751 | Me | Me | A1 | COOH | CH₂CH₂O | J15 | 0 | CH |
| 752 | Me | Me | A1 | COOH | CH₂CH₂O | J16 | 0 | CH |
| 753 | Me | Me | A1 | COOH | CH₂CH₂O | J17 | 0 | CH |
| 754 | Me | Me | A1 | COOH | CH₂CH₂O | J18 | 0 | CH |
| 755 | Me | Me | A1 | COOH | CH₂CH₂O | J19 | 0 | CH |
| 756 | Me | Me | A1 | COOH | CH₂CH₂O | J20 | 0 | CH |
| 757 | Me | Me | A1 | COOH | CH₂CH₂O | J21 | 0 | CH |
| 758 | Me | Me | A1 | COOH | CH₂CH₂O | J22 | 0 | CH |
| 759 | Me | Me | A1 | COOH | CH₂CH₂O | J23 | 0 | CH |
| 760 | Me | Me | A1 | COOH | CH₂CH₂O | J24 | 0 | CH |
| 761 | Me | Me | A1 | COOH | CH₂CH₂O | J25 | 0 | CH |
| 762 | Me | Me | A1 | COOH | CH₂CH₂O | J26 | 0 | CH |
| 763 | Me | Me | A1 | COOH | CH₂CH₂O | J27 | 0 | CH |
| 764 | Me | Me | A1 | COOH | CH₂CH₂O | J28 | 0 | CH |
| 765 | Me | Me | A1 | COOH | CH₂CH₂O | J29 | 0 | CH |
| 766 | Me | Me | A1 | COOH | CH₂CH₂O | J30 | 0 | CH |
| 767 | Me | Me | A1 | COOH | CH₂CH₂O | J31 | 0 | CH |
| 768 | Me | Me | A1 | COOH | CH₂CH₂O | J32 | 0 | CH |
| 769 | Me | Me | A1 | COOH | CH₂CH₂O | J33 | 0 | CH |
| 770 | Me | Me | A1 | COOH | CH₂CH₂O | J34 | 0 | CH |
| 771 | Me | Me | A1 | COOH | CH₂CH₂O | J35 | 0 | CH |
| 772 | Me | Me | A1 | COOH | CH₂CH₂O | J37 | 0 | CH |
| 773 | Me | Me | A1 | COOH | CH₂CH₂O | J39 | 0 | CH |
| 774 | Me | Me | A1 | COOH | CH₂CH₂O | J62 | 0 | CH |
| 775 | Me | Me | A1 | COOH | CH₂CH₂O | J63 | 0 | CH |

TABLE 32

| Compound | | | | | | | | |
|---|---|---|---|---|---|---|---|---|
| No. | R¹ | R² | SCH₂-A | E | G | J | m | X |
| 776 | H | H | A1 | COOH | CH₂S | J1 | 0 | CH |
| 777 | H | H | A1 | COOH | CH₂S | J2 | 0 | CH |
| 778 | H | H | A1 | COOH | CH₂S | J3 | 0 | CH |
| 779 | H | H | A1 | COOH | CH₂S | J4 | 0 | CH |
| 780 | H | H | A1 | COOH | CH₂S | J8 | 0 | CH |
| 781 | H | H | A1 | COOH | CH₂S | J9 | 0 | CH |
| 782 | H | H | A1 | COOH | CH₂S | J10 | 0 | CH |
| 783 | Me | Me | A1 | COOH | CH₂S | J1 | 0 | CH |
| 784 | Me | Me | A1 | COOH | CH₂S | J2 | 0 | CH |
| 785 | Me | Me | A1 | COOH | CH₂S | J3 | 0 | CH |
| 786 | Me | Me | A1 | COOH | CH₂S | J4 | 0 | CH |
| 787 | Me | Me | A1 | COOH | CH₂S | J8 | 0 | CH |
| 788 | Me | Me | A1 | COOH | CH₂S | J9 | 0 | CH |
| 789 | Me | Me | A1 | COOH | CH₂S | J10 | 0 | CH |
| 790 | H | H | A1 | COOH | CH₂SO₂ | J1 | 0 | CH |
| 791 | H | H | A1 | COOH | CH₂SO₂ | J2 | 0 | CH |
| 792 | H | H | A1 | COOH | CH₂SO₂ | J3 | 0 | CH |
| 793 | H | H | A1 | COOH | CH₂SO₂ | J4 | 0 | CH |
| 794 | H | H | A1 | COOH | CH₂SO₂ | J8 | 0 | CH |
| 795 | H | H | A1 | COOH | CH₂SO₂ | J9 | 0 | CH |
| 796 | H | H | A1 | COOH | CH₂SO₂ | J10 | 0 | CH |
| 797 | Me | Me | A1 | COOH | CH₂SO₂ | J1 | 0 | CH |
| 798 | Me | Me | A1 | COOH | CH₂SO₂ | J2 | 0 | CH |
| 799 | Me | Me | A1 | COOH | CH₂SO₂ | J3 | 0 | CH |
| 800 | Me | Me | A1 | COOH | CH₂SO₂ | J4 | 0 | CH |

TABLE 33

| Compound | | | | | | | | |
|---|---|---|---|---|---|---|---|---|
| No. | R¹ | R² | SCH₂-A | E | G | J | m | X |
| 801 | Me | Me | A1 | COOH | CH₂SO₂ | J8 | 0 | CH |
| 802 | Me | Me | A1 | COOH | CH₂SO₂ | J9 | 0 | CH |
| 803 | Me | Me | A1 | COOH | CH₂SO₂ | J10 | 0 | CH |
| 804 | Me | Me | A1 | COOH | CH₂ | J81 | 0 | CH |
| 805 | Me | Me | A1 | COOH | CH₂ | J82 | 0 | CH |
| 806 | Me | Me | A1 | COOH | CH₂ | J83 | 0 | CH |
| 807 | Me | Me | A1 | COOH | CH₂ | J84 | 0 | CH |
| 808 | Me | Me | A1 | COOH | CH₂ | J85 | 0 | CH |
| 809 | H | H | A1 | COOH | CH₂ | J81 | 0 | CH |
| 810 | H | H | A1 | COOH | CH₂ | J82 | 0 | CH |
| 811 | H | H | A1 | COOH | CH₂ | J83 | 0 | CH |
| 812 | H | H | A1 | COOH | CH₂ | J84 | 0 | CH |
| 813 | H | H | A1 | COOH | CH₂ | J85 | 0 | CH |
| 814 | Me | Me | A1 | COOH | CH₂CH₂ | J1 | 1 | CH |
| 815 | Me | Me | A1 | COOH | CH₂ | J1 | 1 | CH |
| 816 | Me | Me | A1 | COOH | CH₂ | J37 | 1 | CH |
| 817 | Me | Me | A1 | COOH | CH₂ | J39 | 1 | CH |
| 818 | Me | Me | A1 | COOH | CH₂ | J50 | 1 | CH |
| 819 | Me | Me | A1 | COOH | CH₂ | J63 | 1 | CH |
| 820 | Me | Me | A1 | COOH | CH₂ | J64 | 1 | CH |
| 821 | Me | Me | A1 | COOH | CH₂ | J65 | 1 | CH |
| 822 | H | H | A1 | COOH | CH₂ | J37 | 1 | CH |
| 823 | H | H | A1 | COOH | CH₂ | J39 | 1 | CH |
| 824 | H | H | A1 | COOH | CH₂ | J50 | 1 | CH |
| 825 | H | H | A1 | COOH | CH₂ | J63 | 1 | CH |

TABLE 34

| Compound | | | | | | | | |
|---|---|---|---|---|---|---|---|---|
| No. | R¹ | R² | SCH₂-A | E | G | J | m | X |
| 826 | H | H | A1 | COOH | CH₂ | J64 | 1 | CH |
| 827 | H | H | A1 | COOH | CH₂ | J65 | 1 | CH |
| 828 | Cl | Cl | A1 | COOH | CH₂ | J37 | 1 | CH |
| 829 | Cl | Cl | A1 | COOH | CH₂ | J39 | 1 | CH |
| 830 | Cl | Cl | A1 | COOH | CH₂ | J50 | 1 | CH |
| 831 | Cl | Cl | A1 | COOH | CH₂ | J63 | 1 | CH |
| 832 | Cl | Cl | A1 | COOH | CH₂ | J64 | 1 | CH |
| 833 | Cl | Cl | A1 | COOH | CH₂ | J65 | 1 | CH |
| 834 | H | H | A4 | COOH | CH₂ | J37 | 1 | CH |
| 835 | H | H | A4 | COOH | CH₂ | J39 | 1 | CH |
| 836 | H | H | A4 | COOH | CH₂ | J50 | 1 | CH |
| 837 | H | H | A4 | COOH | CH₂ | J63 | 1 | CH |
| 838 | H | H | A4 | COOH | CH₂ | J64 | 1 | CH |
| 839 | H | H | A4 | COOH | CH₂ | J65 | 1 | CH |
| 840 | H | H | A11 | COOH | CH₂ | J37 | 1 | CH |
| 841 | H | H | A11 | COOH | CH₂ | J39 | 1 | CH |
| 842 | H | H | A11 | COOH | CH₂ | J50 | 1 | CH |
| 843 | H | H | A11 | COOH | CH₂ | J63 | 1 | CH |
| 844 | H | H | A11 | COOH | CH₂ | J64 | 1 | CH |
| 845 | H | H | A11 | COOH | CH₂ | J65 | 1 | CH |
| 846 | H | H | A18 | COOH | CH₂ | J37 | 1 | CH |
| 847 | H | H | A18 | COOH | CH₂ | J39 | 1 | CH |
| 848 | H | H | A18 | COOH | CH₂ | J50 | 1 | CH |
| 849 | H | H | A18 | COOH | CH₂ | J63 | 1 | CH |
| 850 | H | H | A18 | COOH | CH₂ | J64 | 1 | CH |

TABLE 35

| Compound | | | | | | | | |
|---|---|---|---|---|---|---|---|---|
| No. | R¹ | R² | SCH₂-A | E | G | J | m | X |
| 851 | H | H | A18 | COOH | CH₂ | J65 | 1 | CH |
| 852 | H | H | A20 | COOH | CH₂ | J37 | 1 | CH |
| 853 | H | H | A20 | COOH | CH₂ | J39 | 1 | CH |
| 854 | H | H | A20 | COOH | CH₂ | J50 | 1 | CH |
| 855 | H | H | A20 | COOH | CH₂ | J63 | 1 | CH |
| 856 | H | H | A20 | COOH | CH₂ | J64 | 1 | CH |
| 857 | H | H | A20 | COOH | CH₂ | J65 | 1 | CH |
| 858 | Me | Me | A1 | COOH | CH₂CH₂ | J1 | 2 | CH |
| 859 | Me | Me | A1 | COOH | CH₂ | J1 | 2 | CH |
| 860 | Me | Me | A1 | COOH | CH₂ | J37 | 2 | CH |
| 861 | Me | Me | A1 | COOH | CH₂ | J39 | 2 | CH |
| 862 | Me | Me | A1 | COOH | CH₂ | J50 | 2 | CH |
| 863 | Me | Me | A1 | COOH | CH₂ | J63 | 2 | CH |
| 864 | Me | Me | A1 | COOH | CH₂ | J64 | 2 | CH |
| 865 | Me | Me | A1 | COOH | CH₂ | J65 | 2 | CH |
| 866 | H | H | A1 | COOH | CH₂ | J37 | 2 | CH |
| 867 | H | H | A1 | COOH | CH₂ | J39 | 2 | CH |
| 868 | H | H | A1 | COOH | CH₂ | J50 | 2 | CH |
| 869 | H | H | A1 | COOH | CH₂ | J63 | 2 | CH |
| 870 | H | H | A1 | COOH | CH₂ | J64 | 2 | CH |
| 871 | H | H | A1 | COOH | CH₂ | J65 | 2 | CH |
| 872 | Cl | Cl | A1 | COOH | CH₂ | J37 | 2 | CH |
| 873 | Cl | Cl | A1 | COOH | CH₂ | J39 | 2 | CH |
| 874 | Cl | Cl | A1 | COOH | CH₂ | J50 | 2 | CH |
| 875 | Cl | Cl | A1 | COOH | CH₂ | J63 | 2 | CH |

TABLE 36

| Compound | | | | | | | | |
|---|---|---|---|---|---|---|---|---|
| No. | R¹ | R² | SCH₂-A | E | G | J | m | X |
| 876 | Cl | Cl | A1 | COOH | CH₂ | J64 | 2 | CH |
| 877 | Cl | Cl | A1 | COOH | CH₂ | J65 | 2 | CH |
| 878 | H | H | A1 | COOH | CH₂ | J37 | 2 | N |
| 879 | H | H | A1 | COOH | CH₂ | J39 | 2 | N |
| 880 | H | H | A1 | COOH | CH₂ | J50 | 2 | N |
| 881 | H | H | A1 | COOH | CH₂ | J63 | 2 | N |
| 882 | H | H | A1 | COOH | CH₂ | J64 | 2 | N |
| 883 | H | H | A1 | COOH | CH₂ | J65 | 2 | N |
| 884 | Me | H | A1 | COOH | CH₂ | J37 | 2 | CH |
| 885 | Me | H | A1 | COOH | CH₂ | J63 | 2 | CH |
| 886 | Me | H | A1 | COOH | CH₂ | J64 | 2 | CH |
| 887 | Me | H | A1 | COOH | CH₂ | J65 | 2 | CH |
| 888 | H | H | A4 | COOH | CH₂ | J37 | 2 | CH |
| 889 | H | H | A4 | COOH | CH₂ | J63 | 2 | CH |
| 890 | H | H | A4 | COOH | CH₂ | J64 | 2 | CH |
| 891 | H | H | A4 | COOH | CH₂ | J65 | 2 | CH |
| 892 | Me | Me | A4 | COOH | CH₂ | J37 | 2 | CH |
| 893 | Me | Me | A4 | COOH | CH₂ | J63 | 2 | CH |
| 894 | Me | Me | A4 | COOH | CH₂ | J64 | 2 | CH |
| 895 | Me | Me | A4 | COOH | CH₂ | J65 | 2 | CH |
| 896 | Cl | Cl | A4 | COOH | CH₂ | J37 | 2 | CH |
| 897 | Cl | Cl | A4 | COOH | CH₂ | J63 | 2 | CH |
| 898 | Cl | Cl | A4 | COOH | CH₂ | J64 | 2 | CH |
| 899 | Cl | Cl | A4 | COOH | CH₂ | J65 | 2 | CH |
| 900 | H | H | A4 | COOH | CH₂ | J37 | 2 | N |

TABLE 37

| Compound | | | | | | | | |
|---|---|---|---|---|---|---|---|---|
| No. | R¹ | R² | SCH₂-A | E | G | J | m | X |
| 901 | H | H | A4 | COOH | CH₂ | J63 | 2 | N |
| 902 | H | H | A4 | COOH | CH₂ | J64 | 2 | N |
| 903 | H | H | A4 | COOH | CH₂ | J65 | 2 | N |
| 904 | H | H | A11 | COOH | CH₂ | J37 | 2 | CH |
| 905 | H | H | A11 | COOH | CH₂ | J63 | 2 | CH |
| 906 | H | H | A11 | COOH | CH₂ | J64 | 2 | CH |
| 907 | H | H | A11 | COOH | CH₂ | J65 | 2 | CH |
| 908 | Me | Me | A11 | COOH | CH₂ | J37 | 2 | CH |
| 909 | Me | Me | A11 | COOH | CH₂ | J63 | 2 | CH |
| 910 | Me | Me | A11 | COOH | CH₂ | J64 | 2 | C |
| 911 | Me | Me | A11 | COOH | CH₂ | J65 | 2 | CH |
| 912 | Cl | Cl | A11 | COOH | CH₂ | J37 | 2 | CH |
| 913 | Cl | Cl | A11 | COOH | CH₂ | J63 | 2 | CH |
| 914 | Cl | Cl | A11 | COOH | CH₂ | J64 | 2 | CH |
| 915 | Cl | Cl | A11 | COOH | CH₂ | J65 | 2 | CH |
| 916 | H | H | A11 | COOH | CH₂ | J37 | 2 | N |
| 917 | H | H | A11 | COOH | CH₂ | J63 | 2 | N |
| 918 | H | H | A11 | COOH | CH₂ | J64 | 2 | N |
| 919 | H | H | A11 | COOH | CH₂ | J65 | 2 | N |
| 920 | Me | Me | A18 | COOH | CH₂ | J37 | 2 | CH |
| 921 | Me | Me | A18 | COOH | CH₂ | J63 | 2 | CH |
| 922 | Me | Me | A18 | COOH | CH₂ | J64 | 2 | CH |
| 923 | Me | Me | A18 | COOH | CH₂ | J65 | 2 | CH |
| 924 | H | H | A18 | COOH | CH₂ | J37 | 2 | CH |
| 925 | H | H | A18 | COOH | CH₂ | J63 | 2 | CH |

TABLE 38

| Compound | | | | | | | | |
|---|---|---|---|---|---|---|---|---|
| No. | R¹ | R² | SCH₂-A | E | G | J | m | X |
| 926 | H | H | A18 | COOH | CH₂ | J64 | 2 | CH |
| 927 | H | H | A18 | COOH | CH₂ | J65 | 2 | CH |
| 928 | Cl | Cl | A18 | COOH | CH₂ | J37 | 2 | CH |
| 929 | Cl | Cl | A18 | COOH | CH₂ | J63 | 2 | CH |
| 930 | Cl | Cl | A18 | COOH | CH₂ | J64 | 2 | CH |
| 931 | Cl | Cl | A18 | COOH | CH₂ | J65 | 2 | CH |
| 932 | H | H | A18 | COOH | CH₂ | J37 | 2 | N |
| 933 | H | H | A18 | COOH | CH₂ | J63 | 2 | N |
| 934 | H | H | A18 | COOH | CH₂ | J64 | 2 | N |
| 935 | H | H | A18 | COOH | CH₂ | J65 | 2 | N |
| 936 | Me | Me | A20 | COOH | CH₂ | J37 | 2 | CH |
| 937 | Me | Me | A20 | COOH | CH₂ | J63 | 2 | CH |
| 938 | Me | Me | A20 | COOH | CH₂ | J64 | 2 | CH |
| 939 | Me | Me | A20 | COOH | CH₂ | J65 | 2 | CH |
| 940 | H | H | A20 | COOH | CH₂ | J37 | 2 | CH |
| 941 | H | H | A20 | COOH | CH₂ | J63 | 2 | CH |
| 942 | H | H | A20 | COOH | CH₂ | J64 | 2 | CH |
| 943 | H | H | A20 | COOH | CH₂ | J65 | 2 | CH |
| 944 | Cl | Cl | A20 | COOH | CH₂ | J37 | 2 | CH |
| 945 | Cl | Cl | A20 | COOH | CH₂ | J63 | 2 | CH |
| 946 | Cl | Cl | A20 | COOH | CH₂ | J64 | 2 | CH |
| 947 | Cl | Cl | A20 | COOH | CH₂ | J65 | 2 | CH |
| 948 | H | H | A20 | COOH | CH₂ | J37 | 2 | N |
| 949 | H | H | A20 | COOH | CH₂ | J63 | 2 | N |
| 950 | H | H | A20 | COOH | CH₂ | J64 | 2 | N |

TABLE 39

| Compound | | | | | | | | |
|---|---|---|---|---|---|---|---|---|
| No. | R¹ | R² | SCH₂-A | E | G | J | m | X |
| 951 | H | H | A20 | COOH | CH₂ | J65 | 2 | N |
| 952 | Me | Me | A1 | tetrazol | CH₂ | J37 | 0 | CH |
| 953 | Me | Me | A1 | tetrazol | CH₂ | J63 | 0 | CH |
| 954 | Me | Me | A1 | tetrazol | CH₂ | J64 | 0 | CH |
| 955 | Me | Me | A1 | tetrazol | CH₂ | J65 | 0 | CH |
| 956 | H | H | A1 | tetrazol | CH₂ | J37 | 0 | CH |
| 957 | H | H | A1 | tetrazol | CH₂ | J63 | 0 | CH |
| 958 | H | H | A1 | tetrazol | CH₂ | J64 | 0 | CH |
| 959 | H | H | A1 | tetrazol | CH₂ | J65 | 0 | CH |
| 960 | Cl | Cl | A1 | tetrazol | CH₂ | J37 | 0 | CH |
| 961 | Cl | Cl | A1 | tetrazol | CH₂ | J63 | 0 | CH |
| 962 | Cl | Cl | A1 | tetrazol | CH₂ | J64 | 0 | CH |
| 963 | Cl | Cl | A1 | tetrazol | CH₂ | J65 | 0 | CH |
| 964 | H | H | A1 | tetrazol | CH₂ | J37 | 0 | N |
| 965 | H | H | A1 | tetrazol | CH₂ | J63 | 0 | N |
| 966 | H | H | A1 | tetrazol | CH₂ | J64 | 0 | N |
| 967 | H | H | A1 | tetrazol | CH₂ | J65 | 0 | N |
| 968 | H | H | A4 | tetrazol | CH₂ | J37 | 0 | CH |
| 969 | H | H | A4 | tetrazol | CH₂ | J63 | 0 | CH |
| 970 | H | H | A4 | tetrazol | CH₂ | J64 | 0 | CH |
| 971 | H | H | A4 | tetrazol | CH₂ | J65 | 0 | CH |
| 972 | H | H | A18 | tetrazol | CH₂ | J37 | 0 | CH |
| 973 | H | H | A18 | tetrazol | CH₂ | J63 | 0 | CH |
| 974 | H | H | A18 | tetrazol | CH₂ | J64 | 0 | CH |
| 975 | H | H | A18 | tetrazol | CH₂ | J65 | 0 | CH |

TABLE 40

| Compound | | | | | | | | |
|---|---|---|---|---|---|---|---|---|
| No. | R¹ | R² | SCH₂-A | E | G | J | m | X |
| 976 | Me | Me | A19 | tetrazol | CH₂ | J37 | 0 | CH |
| 977 | Me | Me | A19 | tetrazol | CH₂ | J63 | 0 | CH |
| 978 | Me | Me | A19 | tetrazol | CH₂ | J64 | 0 | CH |
| 979 | Me | Me | A19 | tetrazol | CH₂ | J65 | 0 | CH |
| 980 | H | H | A19 | tetrazol | CH₂ | J37 | 0 | CH |
| 981 | H | H | A19 | tetrazol | CH₂ | J63 | 0 | CH |
| 982 | H | H | A19 | tetrazol | CH₂ | J64 | 0 | CH |
| 983 | H | H | A19 | tetrazol | CH₂ | J65 | 0 | CH |
| 984 | Me | Me | A20 | tetrazol | CH₂ | J37 | 0 | CH |
| 985 | Me | Me | A20 | tetrazol | CH₂ | J63 | 0 | CH |
| 986 | Me | Me | A20 | tetrazol | CH₂ | J64 | 0 | CH |
| 987 | Me | Me | A20 | tetrazol | CH₂ | J65 | 0 | CH |
| 988 | H | H | A20 | tetrazol | CH₂ | J37 | 0 | CH |
| 989 | H | H | A20 | tetrazol | CH₂ | J63 | 0 | CH |
| 990 | H | H | A20 | tetrazol | CH₂ | J64 | 0 | CH |
| 991 | H | H | A20 | tetrazol | CH₂ | J65 | 0 | CH |

TABLE 41

| Compound No. | R₁ | R₂ | S—CH₂-A | E | G | J | m | X |
|---|---|---|---|---|---|---|---|---|
| 992 | H | H | A22 | COOH | CH₂ | J86 | 0 | CH |
| 993 | H | H | A22 | COOH | CH₂ | J65 | 0 | CH |
| 994 | H | H | A22 | COOH | CH₂ | J87 | 0 | CH |
| 995 | H | H | A22 | COOH | CH₂ | J88 | 0 | CH |
| 996 | H | H | A22 | COOH | CH₂ | J89 | 0 | CH |
| 997 | H | H | A22 | COOH | CH₂ | J90 | 0 | CH |
| 998 | H | H | A22 | COOH | CH₂ | J91 | 0 | CH |
| 999 | H | H | A22 | COOH | CH₂ | J92 | 0 | CH |
| 1000 | H | H | A22 | COOH | CH₂ | J93 | 0 | CH |
| 1001 | H | H | A22 | COOH | CH₂ | J94 | 0 | CH |
| 1002 | H | H | A22 | COOH | CH₂ | J95 | 0 | CH |
| 1003 | H | H | A22 | COOH | CH₂ | J98 | 0 | CH |
| 1004 | H | H | A22 | COOH | CH₂ | J99 | 0 | CH |
| 1005 | H | H | A22 | COOH | CH₂ | J100 | 0 | CH |
| 1006 | H | H | A22 | COOH | CH₂ | J101 | 0 | CH |
| 1007 | H | H | A22 | COOH | CH₂ | J102 | 0 | CH |
| 1008 | H | H | A22 | COOH | CH₂ | J103 | 0 | CH |
| 1009 | H | H | A22 | COOH | CH₂ | J64 | 0 | CH |
| 1010 | H | H | A22 | COOH | CH₂ | J104 | 0 | CH |
| 1011 | H | H | A22 | COOH | CH₂ | J105 | 0 | CH |
| 1012 | H | H | A22 | COOH | CH₂ | J106 | 0 | CH |
| 1013 | H | H | A22 | COOH | CH₂ | J107 | 0 | CH |
| 1014 | H | H | A22 | COOH | CH₂ | J108 | 0 | CH |
| 1015 | H | H | A22 | COOH | CH₂ | J109 | 0 | CH |
| 1016 | H | H | A22 | COOH | CH₂ | J110 | 0 | CH |
| 1017 | H | H | A22 | COOH | CH₂ | J111 | 0 | CH |

TABLE 42

| Compound No. | R₁ | R₂ | S—CH₂-A | E | G | J | m | X |
|---|---|---|---|---|---|---|---|---|
| 1018 | H | H | A22 | COOH | CH₂ | J112 | 0 | CH |
| 1019 | H | H | A22 | COOH | CH₂ | J113 | 0 | CH |
| 1020 | H | H | A22 | COOH | CH₂ | J114 | 0 | CH |
| 1021 | H | H | A20 | COOH | CH₂ | J86 | 0 | CH |
| 1023 | H | H | A20 | COOH | CH₂ | J87 | 0 | CH |
| 1024 | H | H | A20 | COOH | CH₂ | J88 | 0 | CH |
| 1025 | H | H | A20 | COOH | CH₂ | J89 | 0 | CH |
| 1026 | H | H | A20 | COOH | CH₂ | J90 | 0 | CH |
| 1027 | H | H | A20 | COOH | CH₂ | J91 | 0 | CH |
| 1028 | H | H | A20 | COOH | CH₂ | J92 | 0 | CH |
| 1029 | H | H | A20 | COOH | CH₂ | J93 | 0 | CH |
| 1030 | H | H | A20 | COOH | CH₂ | J94 | 0 | CH |
| 1031 | H | H | A20 | COOH | CH₂ | J95 | 0 | CH |
| 1032 | H | H | A20 | COOH | CH₂ | J98 | 0 | CH |
| 1033 | H | H | A20 | COOH | CH₂ | J99 | 0 | CH |
| 1034 | H | H | A20 | COOH | CH₂ | J100 | 0 | CH |
| 1035 | H | H | A20 | COOH | CH₂ | J101 | 0 | CH |
| 1036 | H | H | A20 | COOH | CH₂ | J102 | 0 | CH |
| 1037 | H | H | A20 | COOH | CH₂ | J103 | 0 | CH |
| 1039 | H | H | A20 | COOH | CH₂ | J104 | 0 | CH |
| 1040 | H | H | A20 | COOH | CH₂ | J105 | 0 | CH |
| 1041 | H | H | A20 | COOH | CH₂ | J106 | 0 | CH |
| 1042 | H | H | A20 | COOH | CH₂ | J107 | 0 | CH |

TABLE 43

| Compound No. | R₁ | R₂ | S—CH₂-A | E | G | J | m | X |
|---|---|---|---|---|---|---|---|---|
| 1043 | H | H | A20 | COOH | CH₂ | J108 | 0 | CH |
| 1044 | H | H | A20 | COOH | CH₂ | J109 | 0 | CH |
| 1045 | H | H | A20 | COOH | CH₂ | J110 | 0 | CH |
| 1046 | H | H | A20 | COOH | CH₂ | J111 | 0 | CH |
| 1047 | H | H | A20 | COOH | CH₂ | J112 | 0 | CH |
| 1048 | H | H | A20 | COOH | CH₂ | J113 | 0 | CH |
| 1049 | H | H | A20 | COOH | CH₂ | J114 | 0 | CH |
| 1050 | H | H | A21 | COOH | CH₂ | J86 | 0 | CH |
| 1051 | H | H | A21 | COOH | CH₂ | J87 | 0 | CH |
| 1052 | H | H | A21 | COOH | CH₂ | J88 | 0 | CH |
| 1053 | H | H | A21 | COOH | CH₂ | J89 | 0 | CH |
| 1054 | H | H | A21 | COOH | CH₂ | J90 | 0 | CH |
| 1055 | H | H | A21 | COOH | CH₂ | J91 | 0 | CH |
| 1056 | H | H | A21 | COOH | CH₂ | J92 | 0 | CH |
| 1057 | H | H | A21 | COOH | CH₂ | J93 | 0 | CH |
| 1058 | H | H | A21 | COOH | CH₂ | J94 | 0 | CH |
| 1059 | H | H | A21 | COOH | CH₂ | J95 | 0 | CH |
| 1060 | H | H | A21 | COOH | CH₂ | J98 | 0 | CH |
| 1061 | H | H | A21 | COOH | CH₂ | J99 | 0 | CH |
| 1062 | H | H | A21 | COOH | CH₂ | J100 | 0 | CH |
| 1063 | H | H | A21 | COOH | CH₂ | J101 | 0 | CH |
| 1064 | H | H | A21 | COOH | CH₂ | J102 | 0 | CH |
| 1065 | H | H | A21 | COOH | CH₂ | J103 | 0 | CH |
| 1066 | H | H | A21 | COOH | CH₂ | J64 | 0 | CH |
| 1067 | H | H | A21 | COOH | CH₂ | J104 | 0 | CH |

TABLE 44

| Compound No. | R₁ | R₂ | S—CH₂-A | E | G | J | m | X |
|---|---|---|---|---|---|---|---|---|
| 1068 | H | H | A21 | COOH | CH₂ | J105 | 0 | CH |
| 1069 | H | H | A21 | COOH | CH₂ | J106 | 0 | CH |
| 1070 | H | H | A21 | COOH | CH₂ | J107 | 0 | CH |
| 1071 | H | H | A21 | COOH | CH₂ | J108 | 0 | CH |
| 1072 | H | H | A21 | COOH | CH₂ | J109 | 0 | CH |
| 1073 | H | H | A21 | COOH | CH₂ | J110 | 0 | CH |
| 1074 | H | H | A21 | COOH | CH₂ | J111 | 0 | CH |
| 1075 | H | H | A21 | COOH | CH₂ | J112 | 0 | CH |
| 1076 | H | H | A21 | COOH | CH₂ | J113 | 0 | CH |
| 1077 | H | H | A21 | COOH | CH₂ | J114 | 0 | CH |
| 1078 | MeO | H | A22 | COOH | CH₂ | J86 | 0 | CH |
| 1079 | MeO | H | A22 | COOH | CH₂ | J65 | 0 | CH |
| 1080 | MeO | H | A22 | COOH | CH₂ | J87 | 0 | CH |
| 1081 | MeO | H | A22 | COOH | CH₂ | J88 | 0 | CH |
| 1082 | MeO | H | A22 | COOH | CH₂ | J89 | 0 | CH |
| 1083 | MeO | H | A22 | COOH | CH₂ | J90 | 0 | CH |
| 1084 | MeO | H | A22 | COOH | CH₂ | J91 | 0 | CH |
| 1085 | MeO | H | A22 | COOH | CH₂ | J92 | 0 | CH |
| 1086 | MeO | H | A22 | COOH | CH₂ | J93 | 0 | CH |
| 1087 | MeO | H | A22 | COOH | CH₂ | J94 | 0 | CH |
| 1088 | MeO | H | A22 | COOH | CH₂ | J95 | 0 | CH |
| 1089 | MeO | H | A22 | COOH | CH₂ | J98 | 0 | CH |
| 1090 | MeO | H | A22 | COOH | CH₂ | J99 | 0 | CH |
| 1091 | MeO | H | A22 | COOH | CH₂ | J100 | 0 | CH |
| 1092 | MeO | H | A22 | COOH | CH₂ | J101 | 0 | CH |
| 1093 | MeO | H | A22 | COOH | CH₂ | J102 | 0 | CH |

TABLE 45

| Compound No. | R₁ | R₂ | S—CH₂-A | E | G | J | m | X |
|---|---|---|---|---|---|---|---|---|
| 1094 | MeO | H | A22 | COOH | CH₂ | J103 | 0 | CH |
| 1095 | MeO | H | A22 | COOH | CH₂ | J64 | 0 | CH |
| 1096 | MeO | H | A22 | COOH | CH₂ | J104 | 0 | CH |
| 1097 | MeO | H | A22 | COOH | CH₂ | J105 | 0 | CH |
| 1098 | MeO | H | A22 | COOH | CH₂ | J106 | 0 | CH |
| 1099 | MeO | H | A22 | COOH | CH₂ | J107 | 0 | CH |
| 1100 | MeO | H | A22 | COOH | CH₂ | J108 | 0 | CH |
| 1101 | MeO | H | A22 | COOH | CH₂ | J109 | 0 | CH |
| 1102 | MeO | H | A22 | COOH | CH₂ | J110 | 0 | CH |
| 1103 | MeO | H | A22 | COOH | CH₂ | J111 | 0 | CH |
| 1104 | MeO | H | A22 | COOH | CH₂ | J112 | 0 | CH |
| 1105 | MeO | H | A22 | COOH | CH₂ | J113 | 0 | CH |
| 1106 | MeO | H | A22 | COOH | CH₂ | J114 | 0 | CH |
| 1107 | MeO | H | A20 | COOH | CH₂ | J86 | 0 | CH |
| 1108 | MeO | H | A20 | COOH | CH₂ | J87 | 0 | CH |
| 1109 | MeO | H | A20 | COOH | CH₂ | J88 | 0 | CH |
| 1110 | MeO | H | A20 | COOH | CH₂ | J89 | 0 | CH |
| 1111 | MeO | H | A20 | COOH | CH₂ | J90 | 0 | CH |
| 1112 | MeO | H | A20 | COOH | CH₂ | J91 | 0 | CH |
| 1113 | MeO | H | A20 | COOH | CH₂ | J92 | 0 | CH |
| 1114 | MeO | H | A20 | COOH | CH₂ | J93 | 0 | CH |
| 1115 | MeO | H | A20 | COOH | CH₂ | J94 | 0 | CH |
| 1116 | MeO | H | A20 | COOH | CH₂ | J95 | 0 | CH |
| 1117 | MeO | H | A20 | COOH | CH₂ | J98 | 0 | CH |
| 1118 | MeO | H | A20 | COOH | CH₂ | J99 | 0 | CH |
| 1119 | MeO | H | A20 | COOH | CH₂ | J100 | 0 | CH |

TABLE 46

| Compound No. | R₁ | R₂ | S—CH₂-A | E | G | J | m | X |
|---|---|---|---|---|---|---|---|---|
| 1120 | MeO | H | A20 | COOH | CH₂ | J101 | 0 | CH |
| 1121 | MeO | H | A20 | COOH | CH₂ | J102 | 0 | CH |
| 1122 | MeO | H | A20 | COOH | CH₂ | J103 | 0 | CH |
| 1124 | MeO | H | A20 | COOH | CH₂ | J104 | 0 | CH |
| 1125 | MeO | H | A20 | COOH | CH₂ | J105 | 0 | CH |
| 1126 | MeO | H | A20 | COOH | CH₂ | J106 | 0 | CH |
| 1127 | MeO | H | A20 | COOH | CH₂ | J107 | 0 | CH |
| 1128 | MeO | H | A20 | COOH | CH₂ | J108 | 0 | CH |
| 1129 | MeO | H | A20 | COOH | CH₂ | J109 | 0 | CH |
| 1130 | MeO | H | A20 | COOH | CH₂ | J110 | 0 | CH |
| 1131 | MeO | H | A20 | COOH | CH₂ | J111 | 0 | CH |
| 1132 | MeO | H | A20 | COOH | CH₂ | J112 | 0 | CH |
| 1133 | MeO | H | A20 | COOH | CH₂ | J113 | 0 | CH |
| 1134 | MeO | H | A20 | COOH | CH₂ | J114 | 0 | CH |
| 1135 | MeO | H | A21 | COOH | CH₂ | J86 | 0 | CH |
| 1136 | MeO | H | A21 | COOH | CH₂ | J65 | 0 | CH |
| 1137 | MeO | H | A21 | COOH | CH₂ | J87 | 0 | CH |
| 1138 | MeO | H | A21 | COOH | CH₂ | J88 | 0 | CH |
| 1139 | MeO | H | A21 | COOH | CH₂ | J89 | 0 | CH |
| 1140 | MeO | H | A21 | COOH | CH₂ | J90 | 0 | CH |
| 1141 | MeO | H | A21 | COOH | CH₂ | J91 | 0 | CH |
| 1142 | MeO | H | A21 | COOH | CH₂ | J92 | 0 | CH |
| 1143 | MeO | H | A21 | COOH | CH₂ | J93 | 0 | CH |
| 1144 | MeO | H | A21 | COOH | CH₂ | J94 | 0 | CH |

TABLE 47

| Compound No. | R₁ | R₂ | S—CH₂-A | E | G | J | m | X |
|---|---|---|---|---|---|---|---|---|
| 1145 | MeO | H | A21 | COOH | CH₂ | J95 | 0 | CH |
| 1146 | MeO | H | A21 | COOH | CH₂ | J98 | 0 | CH |
| 1147 | MeO | H | A21 | COOH | CH₂ | J99 | 0 | CH |
| 1148 | MeO | H | A21 | COOH | CH₂ | J100 | 0 | CH |
| 1149 | MeO | H | A21 | COOH | CH₂ | J101 | 0 | CH |
| 1150 | MeO | H | A21 | COOH | CH₂ | J102 | 0 | CH |
| 1151 | MeO | H | A21 | COOH | CH₂ | J103 | 0 | CH |
| 1152 | MeO | H | A21 | COOH | CH₂ | J64 | 0 | CH |
| 1153 | MeO | H | A21 | COOH | CH₂ | J104 | 0 | CH |
| 1154 | MeO | H | A21 | COOH | CH₂ | J105 | 0 | CH |
| 1155 | MeO | H | A21 | COOH | CH₂ | J106 | 0 | CH |
| 1156 | MeO | H | A21 | COOH | CH₂ | J107 | 0 | CH |
| 1157 | MeO | H | A21 | COOH | CH₂ | J108 | 0 | CH |
| 1158 | MeO | H | A21 | COOH | CH₂ | J109 | 0 | CH |
| 1159 | MeO | H | A21 | COOH | CH₂ | J110 | 0 | CH |
| 1160 | MeO | H | A21 | COOH | CH₂ | J111 | 0 | CH |
| 1161 | MeO | H | A21 | COOH | CH₂ | J112 | 0 | CH |
| 1162 | MeO | H | A21 | COOH | CH₂ | J113 | 0 | CH |
| 1163 | MeO | H | A21 | COOH | CH₂ | J114 | 0 | CH |
| 1164 | CN | H | A22 | COOH | CH₂ | J86 | 0 | CH |
| 1165 | CN | H | A22 | COOH | CH₂ | J65 | 0 | CH |
| 1166 | CN | H | A22 | COOH | CH₂ | J87 | 0 | CH |
| 1167 | CN | H | A22 | COOH | CH₂ | J88 | 0 | CH |
| 1168 | CN | H | A22 | COOH | CH₂ | J89 | 0 | CH |
| 1169 | CN | H | A22 | COOH | CH₂ | J90 | 0 | CH |

TABLE 48

| Compound No. | R₁ | R₂ | S—CH₂-A | E | G | J | m | X |
|---|---|---|---|---|---|---|---|---|
| 1170 | CN | H | A22 | COOH | CH₂ | J91 | 0 | CH |
| 1171 | CN | H | A22 | COOH | CH₂ | J92 | 0 | CH |
| 1172 | CN | H | A22 | COOH | CH₂ | J93 | 0 | CH |
| 1173 | CN | H | A22 | COOH | CH₂ | J94 | 0 | CH |
| 1174 | CN | H | A22 | COOH | CH₂ | J95 | 0 | CH |
| 1175 | CN | H | A22 | COOH | CH₂ | J98 | 0 | CH |
| 1176 | CN | H | A22 | COOH | CH₂ | J99 | 0 | CH |
| 1177 | CN | H | A22 | COOH | CH₂ | J100 | 0 | CH |
| 1178 | CN | H | A22 | COOH | CH₂ | J101 | 0 | CH |
| 1179 | CN | H | A22 | COOH | CH₂ | J102 | 0 | CH |
| 1180 | CN | H | A22 | COOH | CH₂ | J103 | 0 | CH |
| 1181 | CN | H | A22 | COOH | CH₂ | J64 | 0 | CH |
| 1182 | CN | H | A22 | COOH | CH₂ | J104 | 0 | CH |
| 1183 | CN | H | A22 | COOH | CH₂ | J105 | 0 | CH |
| 1184 | CN | H | A22 | COOH | CH₂ | J106 | 0 | CH |
| 1185 | CN | H | A22 | COOH | CH₂ | J107 | 0 | CH |
| 1186 | CN | H | A22 | COOH | CH₂ | J108 | 0 | CH |
| 1187 | CN | H | A22 | COOH | CH₂ | J109 | 0 | CH |
| 1188 | CN | H | A22 | COOH | CH₂ | J110 | 0 | CH |
| 1189 | CN | H | A22 | COOH | CH₂ | J111 | 0 | CH |
| 1190 | CN | H | A22 | COOH | CH₂ | J112 | 0 | CH |
| 1191 | CN | H | A22 | COOH | CH₂ | J113 | 0 | CH |
| 1192 | CN | H | A22 | COOH | CH₂ | J114 | 0 | CH |
| 1193 | CN | H | A20 | COOH | CH₂ | J86 | 0 | CH |

TABLE 49

| Compound No. | R₁ | R₂ | S—CH₂-A | E | G | J | m | X |
|---|---|---|---|---|---|---|---|---|
| 1194 | CN | H | A20 | COOH | CH₂ | J87 | 0 | CH |
| 1195 | CN | H | A20 | COOH | CH₂ | J88 | 0 | CH |
| 1196 | CN | H | A20 | COOH | CH₂ | J89 | 0 | CH |
| 1197 | CN | H | A20 | COOH | CH₂ | J90 | 0 | CH |
| 1198 | CN | H | A20 | COOH | CH₂ | J91 | 0 | CH |
| 1199 | CN | H | A20 | COOH | CH₂ | J92 | 0 | CH |
| 1200 | CN | H | A20 | COOH | CH₂ | J93 | 0 | CH |
| 1201 | CN | H | A20 | COOH | CH₂ | J94 | 0 | CH |
| 1202 | CN | H | A20 | COOH | CH₂ | J95 | 0 | CH |
| 1203 | CN | H | A20 | COOH | CH₂ | J98 | 0 | CH |
| 1204 | CN | H | A20 | COOH | CH₂ | J99 | 0 | CH |
| 1205 | CN | H | A20 | COOH | CH₂ | J100 | 0 | CH |
| 1206 | CN | H | A20 | COOH | CH₂ | J101 | 0 | CH |
| 1207 | CN | H | A20 | COOH | CH₂ | J102 | 0 | CH |
| 1208 | CN | H | A20 | COOH | CH₂ | J103 | 0 | CH |
| 1210 | CN | H | A20 | COOH | CH₂ | J104 | 0 | CH |
| 1211 | CN | H | A20 | COOH | CH₂ | J105 | 0 | CH |
| 1212 | CN | H | A20 | COOH | CH₂ | J106 | 0 | CH |
| 1213 | CN | H | A20 | COOH | CH₂ | J107 | 0 | CH |
| 1214 | CN | H | A20 | COOH | CH₂ | J108 | 0 | CH |
| 1215 | CN | H | A20 | COOH | CH₂ | J109 | 0 | CH |
| 1216 | CN | H | A20 | COOH | CH₂ | J110 | 0 | CH |
| 1217 | CN | H | A20 | COOH | CH₂ | J111 | 0 | CH |
| 1218 | CN | H | A20 | COOH | CH₂ | J112 | 0 | CH |

TABLE 50

| Compound No. | R₁ | R₂ | S—CH₂-A | E | G | J | m | X |
|---|---|---|---|---|---|---|---|---|
| 1219 | CN | H | A20 | COOH | CH₂ | J113 | 0 | CH |
| 1220 | CN | H | A20 | COOH | CH₂ | J114 | 0 | CH |
| 1221 | CN | H | A21 | COOH | CH₂ | J86 | 0 | CH |
| 1222 | CN | H | A21 | COOH | CH₂ | J65 | 0 | CH |
| 1223 | CN | H | A21 | COOH | CH₂ | J87 | 0 | CH |
| 1224 | CN | H | A21 | COOH | CH₂ | J88 | 0 | CH |
| 1225 | CN | H | A21 | COOH | CH₂ | J99 | 0 | CH |
| 1226 | CN | H | A21 | COOH | CH₂ | J90 | 0 | CH |
| 1227 | CN | H | A21 | COOH | CH₂ | J91 | 0 | CH |
| 1228 | CN | H | A21 | COOH | CH₂ | J92 | 0 | CH |
| 1229 | CN | H | A21 | COOH | CH₂ | J93 | 0 | CH |
| 1230 | CN | H | A21 | COOH | CH₂ | J94 | 0 | CH |
| 1231 | CN | H | A21 | COOH | CH₂ | J95 | 0 | CH |
| 1232 | CN | H | A21 | COOH | CH₂ | J98 | 0 | CH |
| 1233 | CN | H | A21 | COOH | CH₂ | J99 | 0 | CH |
| 1234 | CN | H | A21 | COOH | CH₂ | J100 | 0 | CH |
| 1235 | CN | H | A21 | COOH | CH₂ | J101 | 0 | CH |
| 1236 | CN | H | A21 | COOH | CH₂ | J102 | 0 | CH |
| 1237 | CN | H | A21 | COOH | CH₂ | J103 | 0 | CH |
| 1238 | CN | H | A21 | COOH | CH₂ | J64 | 0 | CH |
| 1239 | CN | H | A21 | COOH | CH₂ | J104 | 0 | CH |
| 1240 | CN | H | A21 | COOH | CH₂ | J105 | 0 | CH |
| 1241 | CN | H | A21 | COOH | CH₂ | J106 | 0 | CH |
| 1242 | CN | H | A21 | COOH | CH₂ | J107 | 0 | CH |
| 1243 | CN | H | A21 | COOH | CH₂ | J108 | 0 | CH |

TABLE 51

| Compound No. | R₁ | R₂ | S—CH₂-A | E | G | J | m | X |
|---|---|---|---|---|---|---|---|---|
| 1244 | CN | H | A21 | COOH | CH₂ | J109 | 0 | CH |
| 1245 | CN | H | A21 | COOH | CH₂ | J110 | 0 | CH |
| 1246 | CN | H | A21 | COOH | CH₂ | J111 | 0 | CH |
| 1247 | CN | H | A21 | COOH | CH₂ | J112 | 0 | CH |
| 1248 | CN | H | A21 | COOH | CH₂ | J113 | 0 | CH |
| 1249 | CN | H | A21 | COOH | CH₂ | J114 | 0 | CH |
| 1250 | Me | H | A22 | COOH | CH₂ | J86 | 0 | CH |
| 1251 | Me | H | A22 | COOH | CH₂ | J65 | 0 | CH |
| 1252 | Me | H | A22 | COOH | CH₂ | J87 | 0 | CH |
| 1253 | Me | H | A22 | COOH | CH₂ | J88 | 0 | CH |
| 1254 | Me | H | A22 | COOH | CH₂ | J89 | 0 | CH |
| 1255 | Me | H | A22 | COOH | CH₂ | J90 | 0 | CH |
| 1256 | Me | H | A22 | COOH | CH₂ | J91 | 0 | CH |
| 1257 | Me | H | A22 | COOH | CH₂ | J92 | 0 | CH |
| 1258 | Me | H | A22 | COOH | CH₂ | J93 | 0 | CH |
| 1259 | Me | H | A22 | COOH | CH₂ | J94 | 0 | CH |
| 1260 | Me | H | A22 | COOH | CH₂ | J95 | 0 | CH |
| 1261 | Me | H | A22 | COOH | CH₂ | J98 | 0 | CH |
| 1262 | Me | H | A22 | COOH | CH₂ | J99 | 0 | CH |
| 1263 | Me | H | A22 | COOH | CH₂ | J100 | 0 | CH |
| 1264 | Me | H | A22 | COOH | CH₂ | J101 | 0 | CH |
| 1265 | Me | H | A22 | COOH | CH₂ | J102 | 0 | CH |
| 1266 | Me | H | A22 | COOH | CH₂ | J103 | 0 | CH |
| 1267 | Me | H | A22 | COOH | CH₂ | J64 | 0 | CH |
| 1268 | Me | H | A22 | COOH | CH₂ | J104 | 0 | CH |

TABLE 52

| Compound No. | R₁ | R₂ | S—CH₂-A | E | G | J | m | X |
|---|---|---|---|---|---|---|---|---|
| 1269 | Me | H | A22 | COOH | CH₂ | J105 | 0 | CH |
| 1270 | Me | H | A22 | COOH | CH₂ | J106 | 0 | CH |
| 1271 | Me | H | A22 | COOH | CH₂ | J107 | 0 | CH |
| 1272 | Me | H | A22 | COOH | CH₂ | J108 | 0 | CH |
| 1273 | Me | H | A22 | COOH | CH₂ | J109 | 0 | CH |
| 1274 | Me | H | A22 | COOH | CH₂ | J110 | 0 | CH |
| 1275 | Me | H | A22 | COOH | CH₂ | J111 | 0 | CH |
| 1276 | Me | H | A22 | COOH | CH₂ | J112 | 0 | CH |
| 1277 | Me | H | A22 | COOH | CH₂ | J113 | 0 | CH |
| 1278 | Me | H | A22 | COOH | CH₂ | J114 | 0 | CH |
| 1279 | Me | H | A20 | COOH | CH₂ | J86 | 0 | CH |
| 1280 | Me | H | A20 | COOH | CH₂ | J87 | 0 | CH |
| 1281 | Me | H | A20 | COOH | CH₂ | J88 | 0 | CH |
| 1282 | Me | H | A20 | COOH | CH₂ | J89 | 0 | CH |
| 1283 | Me | H | A20 | COOH | CH₂ | J90 | 0 | CH |
| 1284 | Me | H | A20 | COOH | CH₂ | J91 | 0 | CH |
| 1285 | Me | H | A20 | COOH | CH₂ | J92 | 0 | CH |
| 1286 | Me | H | A20 | COOH | CH₂ | J93 | 0 | CH |
| 1287 | Me | H | A20 | COOH | CH₂ | J94 | 0 | CH |
| 1288 | Me | H | A20 | COOH | CH₂ | J95 | 0 | CH |
| 1289 | Me | H | A20 | COOH | CH₂ | J98 | 0 | CH |
| 1290 | Me | H | A20 | COOH | CH₂ | J99 | 0 | CH |
| 1291 | Me | H | A20 | COOH | CH₂ | J100 | 0 | CH |
| 1292 | Me | H | A20 | COOH | CH₂ | J101 | 0 | CH |
| 1293 | Me | H | A20 | COOH | CH₂ | J102 | 0 | CH |

TABLE 53

| Compound No. | R₁ | R₂ | S—CH₂-A | E | G | J | m | X |
|---|---|---|---|---|---|---|---|---|
| 1294 | Me | H | A20 | COOH | CH₂ | J103 | 0 | CH |
| 1296 | Me | H | A20 | COOH | CH₂ | J104 | 0 | CH |
| 1297 | Me | H | A20 | COOH | CH₂ | J105 | 0 | CH |
| 1298 | Me | H | A20 | COOH | CH₂ | J106 | 0 | CH |
| 1299 | Me | H | A20 | COOH | CH₂ | J107 | 0 | CH |
| 1300 | Me | H | A20 | COOH | CH₂ | J108 | 0 | CH |
| 1301 | Me | H | A20 | COOH | CH₂ | J109 | 0 | CH |
| 1302 | Me | H | A20 | COOH | CH₂ | J110 | 0 | CH |
| 1303 | Me | H | A20 | COOH | CH₂ | J111 | 0 | CH |
| 1304 | Me | H | A20 | COOH | CH₂ | J112 | 0 | CH |
| 1305 | Me | H | A20 | COOH | CH₂ | J113 | 0 | CH |
| 1306 | Me | H | A20 | COOH | CH₂ | J114 | 0 | CH |
| 1307 | Me | H | A21 | COOH | CH₂ | J86 | 0 | CH |
| 1308 | Me | H | A21 | COOH | CH₂ | J65 | 0 | CH |
| 1309 | Me | H | A21 | COOH | CH₂ | J87 | 0 | CH |
| 1310 | Me | H | A21 | COOH | CH₂ | J88 | 0 | CH |
| 1311 | Me | H | A21 | COOH | CH₂ | J89 | 0 | CH |
| 1312 | Me | H | A21 | COOH | CH₂ | J90 | 0 | CH |
| 1313 | Me | H | A21 | COOH | CH₂ | J91 | 0 | CH |
| 1314 | Me | H | A21 | COOH | CH₂ | J92 | 0 | CH |
| 1315 | Me | H | A21 | COOH | CH₂ | J93 | 0 | CH |
| 1316 | Me | H | A21 | COOH | CH₂ | J94 | 0 | CH |

TABLE 54

| Compound No. | R₁ | R₂ | S—CH₂-A | E | G | J | m | X |
|---|---|---|---|---|---|---|---|---|
| 1317 | Me | H | A21 | COOH | CH₂ | J95 | 0 | CH |
| 1318 | Me | H | A21 | COOH | CH₂ | J98 | 0 | CH |
| 1319 | Me | H | A21 | COOH | CH₂ | J99 | 0 | CH |
| 1320 | Me | H | A21 | COOH | CH₂ | J100 | 0 | CH |
| 1321 | Me | H | A21 | COOH | CH₂ | J101 | 0 | CH |
| 1322 | Me | H | A21 | COOH | CH₂ | J102 | 0 | CH |
| 1323 | Me | H | A21 | COOH | CH₂ | J103 | 0 | CH |
| 1324 | Me | H | A21 | COOH | CH₂ | J64 | 0 | CH |
| 1325 | Me | H | A21 | COOH | CH₂ | J104 | 0 | CH |
| 1326 | Me | H | A21 | COOH | CH₂ | J105 | 0 | CH |
| 1327 | Me | H | A21 | COOH | CH₂ | J106 | 0 | CH |
| 1328 | Me | H | A21 | COOH | CH₂ | J107 | 0 | CH |
| 1329 | Me | H | A21 | COOH | CH₂ | J108 | 0 | CH |
| 1330 | Me | H | A21 | COOH | CH₂ | J109 | 0 | CH |
| 1331 | Me | H | A21 | COOH | CH₂ | J110 | 0 | CH |
| 1332 | Me | H | A21 | COOH | CH₂ | J111 | 0 | CH |
| 1333 | Me | H | A21 | COOH | CH₂ | J112 | 0 | CH |
| 1334 | Me | H | A21 | COOH | CH₂ | J113 | 0 | CH |
| 1335 | Me | H | A21 | COOH | CH₂ | J114 | 0 | CH |
| 1336 | H | Me | A22 | COOH | CH₂ | J86 | 0 | CH |
| 1336-2 | H | Me | A22 | COOH | CH₂ | J65 | 0 | CH |
| 1337 | H | Me | A22 | COOH | CH₂ | J87 | 0 | CH |
| 1338 | H | Me | A22 | COOH | CH₂ | J88 | 0 | CH |
| 1339 | H | Me | A22 | COOH | CH₂ | J89 | 0 | CH |
| 1340 | H | Me | A22 | COOH | CH₂ | J90 | 0 | CH |

TABLE 55

| Compound No. | R₁ | R₂ | S—CH₂-A | E | G | J | m | X |
|---|---|---|---|---|---|---|---|---|
| 1341 | H | Me | A22 | COOH | CH₂ | J91 | 0 | CH |
| 1342 | H | Me | A22 | COOH | CH₂ | J92 | 0 | CH |
| 1343 | H | Me | A22 | COOH | CH₂ | J93 | 0 | CH |
| 1344 | H | Me | A22 | COOH | CH₂ | J94 | 0 | CH |
| 1345 | H | Me | A22 | COOH | CH₂ | J95 | 0 | CH |
| 1347 | H | Me | A22 | COOH | CH₂ | J98 | 0 | CH |
| 1348 | H | Me | A22 | COOH | CH₂ | J99 | 0 | CH |
| 1349 | H | Me | A22 | COOH | CH₂ | J100 | 0 | CH |
| 1350 | H | Me | A22 | COOH | CH₂ | J101 | 0 | CH |
| 1351 | H | Me | A22 | COOH | CH₂ | J102 | 0 | CH |
| 1352 | H | Me | A22 | COOH | CH₂ | J103 | 0 | CH |
| 1353 | H | Me | A22 | COOH | CH₂ | J64 | 0 | CH |
| 1354 | H | Me | A22 | COOH | CH₂ | J104 | 0 | CH |
| 1355 | H | Me | A22 | COOH | CH₂ | J105 | 0 | CH |
| 1356 | H | Me | A22 | COOH | CH₂ | J106 | 0 | CH |
| 1357 | H | Me | A22 | COOH | CH₂ | J107 | 0 | CH |
| 1358 | H | Me | A22 | COOH | CH₂ | J108 | 0 | CH |
| 1359 | H | Me | A22 | COOH | CH₂ | J109 | 0 | CH |
| 1360 | H | Me | A22 | COOH | CH₂ | J110 | 0 | CH |
| 1361 | H | Me | A22 | COOH | CH₂ | J111 | 0 | CH |
| 1362 | H | Me | A22 | COOH | CH₂ | J112 | 0 | CH |
| 1363 | H | Me | A22 | COOH | CH₂ | J113 | 0 | CH |
| 1364 | H | Me | A22 | COOH | CH₂ | J114 | 0 | CH |
| 1365 | H | Me | A20 | COOH | CH₂ | J86 | 0 | CH |

TABLE 56

| Compound No. | R₁ | R₂ | S—CH₂-A | E | G | J | m | X |
|---|---|---|---|---|---|---|---|---|
| 1366 | H | Me | A20 | COOH | CH₂ | J65 | 0 | CH |
| 1367 | H | Me | A20 | COOH | CH₂ | J87 | 0 | CH |
| 1368 | H | Me | A20 | COOH | CH₂ | J88 | 0 | CH |
| 1369 | H | Me | A20 | COOH | CH₂ | J89 | 0 | CH |
| 1370 | H | Me | A20 | COOH | CH₂ | J90 | 0 | CH |
| 1371 | H | Me | A20 | COOH | CH₂ | J91 | 0 | CH |
| 1372 | H | Me | A20 | COOH | CH₂ | J92 | 0 | CH |
| 1373 | H | Me | A20 | COOH | CH₂ | J93 | 0 | CH |
| 1374 | H | Me | A20 | COOH | CH₂ | J94 | 0 | CH |
| 1375 | H | Me | A20 | COOH | CH₂ | J95 | 0 | CH |
| 1376 | H | Me | A20 | COOH | CH₂ | J98 | 0 | CH |
| 1377 | H | Me | A20 | COOH | CH₂ | J99 | 0 | CH |
| 1378 | H | Me | A20 | COOH | CH₂ | J100 | 0 | CH |
| 1379 | H | Me | A20 | COOH | CH₂ | J101 | 0 | CH |
| 1380 | H | Me | A20 | COOH | CH₂ | J102 | 0 | CH |
| 1381 | H | Me | A20 | COOH | CH₂ | J103 | 0 | CH |
| 1382 | H | Me | A20 | COOH | CH₂ | J64 | 0 | CH |
| 1383 | H | Me | A20 | COOH | CH₂ | J104 | 0 | CH |
| 1384 | H | Me | A20 | COOH | CH₂ | J105 | 0 | CH |
| 1385 | H | Me | A20 | COOH | CH₂ | J106 | 0 | CH |
| 1386 | H | Me | A20 | COOH | CH₂ | J107 | 0 | CH |
| 1387 | H | Me | A20 | COOH | CH₂ | J108 | 0 | CH |
| 1388 | H | Me | A20 | COOH | CH₂ | J109 | 0 | CH |
| 1389 | H | Me | A20 | COOH | CH₂ | J110 | 0 | CH |
| 1390 | H | Me | A20 | COOH | CH₂ | J111 | 0 | CH |

TABLE 57

| Compound No. | R₁ | R₂ | S—CH₂-A | E | G | J | m | X |
|---|---|---|---|---|---|---|---|---|
| 1391 | H | Me | A20 | COOH | CH₂ | J112 | 0 | CH |
| 1392 | H | Me | A20 | COOH | CH₂ | J113 | 0 | CH |
| 1393 | H | Me | A20 | COOH | CH₂ | J114 | 0 | CH |
| 1394 | H | Me | A21 | COOH | CH₂ | J86 | 0 | CH |
| 1395 | H | Me | A21 | COOH | CH₂ | J65 | 0 | CH |
| 1396 | H | Me | A21 | COOH | CH₂ | J87 | 0 | CH |
| 1397 | H | Me | A21 | COOH | CH₂ | J88 | 0 | CH |
| 1398 | H | Me | A21 | COOH | CH₂ | J89 | 0 | CH |
| 1399 | H | Me | A21 | COOH | CH₂ | J90 | 0 | CH |
| 1400 | H | Me | A21 | COOH | CH₂ | J91 | 0 | CH |
| 1401 | H | Me | A21 | COOH | CH₂ | J92 | 0 | CH |
| 1402 | H | Me | A21 | COOH | CH₂ | J93 | 0 | CH |
| 1403 | H | Me | A21 | COOH | CH₂ | J94 | 0 | CH |
| 1404 | H | Me | A21 | COOH | CH₂ | J95 | 0 | CH |
| 1405 | H | Me | A21 | COOH | CH₂ | J98 | 0 | CH |
| 1406 | H | Me | A21 | COOH | CH₂ | J99 | 0 | CH |
| 1407 | H | Me | A21 | COOH | CH₂ | J100 | 0 | CH |
| 1408 | H | Mu | A21 | COOH | CH₂ | J101 | 0 | CH |
| 1409 | H | Me | A21 | COOH | CH₂ | J102 | 0 | CH |
| 1410 | H | Me | A21 | COOH | CH₂ | J103 | 0 | CH |
| 1411 | H | Me | A21 | COOH | CH₂ | J64 | 0 | CH |
| 1412 | H | Me | A21 | COOH | CH₂ | J104 | 0 | CH |
| 1413 | H | Me | A21 | COOH | CH₂ | J105 | 0 | CH |
| 1414 | H | Me | A21 | COOH | CH₂ | J106 | 0 | CH |
| 1415 | H | Me | A21 | COOH | CH₂ | J107 | 0 | CH |

TABLE 58

| Compound No. | R₁ | R₂ | S—CH₂-A | E | G | J | m | X |
|---|---|---|---|---|---|---|---|---|
| 1416 | H | Me | A21 | COOH | CH₂ | J108 | 0 | CH |
| 1417 | H | Me | A21 | COOH | CH₂ | J109 | 0 | CH |
| 1418 | H | Me | A21 | COOH | CH₂ | J110 | 0 | CH |
| 1419 | H | Me | A21 | COOH | CH₂ | J111 | 0 | CH |
| 1420 | H | Me | A21 | COOH | CH₂ | J112 | 0 | CH |
| 1421 | H | Me | A21 | COOH | CH₂ | J113 | 0 | CH |
| 1422 | H | Me | A21 | COOH | CH₂ | J114 | 0 | CH |
| 1423 | Me | Me | A22 | COOH | CH₂ | J86 | 0 | CH |
| 1424 | Me | Me | A22 | COOH | CH₂ | J65 | 0 | CH |
| 1425 | Me | Me | A22 | COOH | CH₂ | J87 | 0 | CH |
| 1426 | Me | Me | A22 | COOH | CH₂ | J88 | 0 | CH |
| 1427 | Me | Me | A22 | COOH | CH₂ | J89 | 0 | CH |
| 1428 | Me | Me | A22 | COOH | CH₂ | J90 | 0 | CH |
| 1429 | Me | Me | A22 | COOH | CH₂ | J91 | 0 | CH |
| 1430 | Me | Me | A22 | COOH | CH₂ | J92 | 0 | CH |
| 1431 | Me | Me | A22 | COOH | CH₂ | J93 | 0 | CH |
| 1432 | Me | Me | A22 | COOH | CH₂ | J94 | 0 | CH |
| 1433 | Me | Me | A22 | COOH | CH₂ | J95 | 0 | CH |
| 1434 | Me | Me | A22 | COOH | CH₂ | J98 | 0 | CH |
| 1435 | Me | Me | A22 | COOH | CH₂ | J99 | 0 | CH |
| 1436 | Me | Me | A22 | COOH | CH₂ | J100 | 0 | CH |
| 1437 | Me | Me | A22 | COOH | CH₂ | J101 | 0 | CH |
| 1438 | Me | Me | A22 | COOH | CH₂ | J102 | 0 | CH |
| 1439 | Me | Me | A22 | COOH | CH₂ | J103 | 0 | CH |
| 1440 | Me | Me | A22 | COOH | CH₂ | J64 | 0 | CH |

TABLE 59

| Compound No. | R₁ | R₂ | S—CH₂-A | E | G | J | m | X |
|---|---|---|---|---|---|---|---|---|
| 1441 | Me | Me | A22 | COOH | CH₂ | J104 | 0 | CH |
| 1442 | Me | Me | A22 | COOH | CH₂ | J105 | 0 | CH |
| 1443 | Me | Me | A22 | COOH | CH₂ | J106 | 0 | CH |
| 1444 | Me | Me | A22 | COOH | CH₂ | J107 | 0 | CH |
| 1445 | Me | Me | A22 | COOH | CH₂ | J108 | 0 | CH |
| 1446 | Me | Me | A22 | COOH | CH₂ | J109 | 0 | CH |
| 1447 | Me | Me | A22 | COOH | CH₂ | J110 | 0 | CH |
| 1448 | Me | Me | A22 | COOH | CH₂ | J111 | 0 | CH |
| 1449 | Me | Me | A22 | COOH | CH₂ | J112 | 0 | CH |
| 1450 | Me | Me | A22 | COOH | CH₂ | J113 | 0 | CH |
| 1451 | Me | Me | A22 | COOH | CH₂ | J114 | 0 | CH |
| 1452 | Me | Me | A20 | COOH | CH₂ | J86 | 0 | CH |
| 1454 | Me | Me | A20 | COOH | CH₂ | J87 | 0 | CH |
| 1455 | Me | Me | A20 | COOH | CH₂ | J88 | 0 | CH |
| 1456 | Me | Me | A20 | COOH | CH₂ | J89 | 0 | CH |
| 1457 | Me | Me | A20 | COOH | CH₂ | J90 | 0 | CH |
| 1458 | Me | Me | A20 | COOH | CH₂ | J91 | 0 | CH |
| 1459 | Me | Me | A20 | COOH | CH₂ | J92 | 0 | CH |
| 1460 | Me | Me | A20 | COOH | CH₂ | J93 | 0 | CH |
| 1461 | Me | Me | A20 | COOH | CH₂ | J94 | 0 | CH |
| 1462 | Me | Me | A20 | COOH | CH₂ | J95 | 0 | CH |
| 1463 | Me | Me | A20 | COOH | CH₂ | J98 | 0 | CH |
| 1464 | Me | Me | A20 | COOH | CH₂ | J99 | 0 | CH |
| 1465 | Me | Me | A20 | COOH | CH₂ | J100 | 0 | CH |

TABLE 60

| Compound No. | R₁ | R₂ | S—CH₂-A | E | G | J | m | X |
|---|---|---|---|---|---|---|---|---|
| 1466 | Me | Me | A20 | COOH | CH₂ | J101 | 0 | CH |
| 1467 | Me | Me | A20 | COOH | CH₂ | J102 | 0 | CH |
| 1468 | Me | Me | A20 | COOH | CH₂ | J103 | 0 | CH |
| 1470 | Me | Me | A20 | COOH | CH₂ | J104 | 0 | CH |
| 1471 | Me | Me | A20 | COOH | CH₂ | J105 | 0 | CH |
| 1472 | Me | Me | A20 | COOH | CH₂ | J106 | 0 | CH |
| 1473 | Me | Me | A20 | COOH | CH₂ | J107 | 0 | CH |
| 1474 | Me | Me | A20 | COOH | CH₂ | J108 | 0 | CH |
| 1475 | Me | Me | A20 | COOH | CH₂ | J109 | 0 | CH |
| 1476 | Me | Me | A20 | COOH | CH₂ | J110 | 0 | CH |
| 1477 | Me | Me | A20 | COOH | CH₂ | J111 | 0 | CH |
| 1478 | Me | Me | A20 | COOH | CH₂ | J112 | 0 | CH |
| 1479 | Me | Me | A20 | COOH | CH₂ | J113 | 0 | CH |
| 1480 | Me | Me | A20 | COOH | CH₂ | J114 | 0 | CH |
| 1481 | Me | Me | A21 | COOH | CH₂ | J86 | 0 | CH |
| 1482 | Me | Me | A21 | COOH | CH₂ | J65 | 0 | CH |
| 1483 | Me | Me | A21 | COOH | CH₂ | J87 | 0 | CH |
| 1484 | Me | Me | A21 | COOH | CH₂ | J88 | 0 | CH |
| 1485 | Me | Me | A21 | COOH | CH₂ | J89 | 0 | CH |
| 1486 | Me | Me | A21 | COOH | CH₂ | J90 | 0 | CH |
| 1487 | Me | Me | A21 | COOH | CH₂ | J91 | 0 | CH |
| 1488 | Me | Me | A21 | COOH | CH₂ | J92 | 0 | CH |
| 1489 | Me | Me | A21 | COOH | CH₂ | J93 | 0 | CH |
| 1490 | Me | Me | A21 | COOH | CH₂ | J94 | 0 | CH |

TABLE 61

| Compound No. | R₁ | R₂ | S—CH₂-A | E | G | J | m | X |
|---|---|---|---|---|---|---|---|---|
| 1491 | Me | Me | A21 | COOH | CH₂ | J95 | 0 | CH |
| 1492 | Me | Me | A21 | COOH | CH₂ | J98 | 0 | CH |
| 1493 | Me | Me | A21 | COOH | CH₂ | J99 | 0 | CH |
| 1494 | Me | Me | A21 | COOH | CH₂ | J100 | 0 | CH |
| 1495 | Me | Me | A21 | COOH | CH₂ | J101 | 0 | CH |
| 1496 | Me | Me | A21 | COOH | CH₂ | J102 | 0 | CH |
| 1497 | Me | Me | A21 | COOH | CH₂ | J103 | 0 | CH |
| 1498 | Me | Me | A21 | COOH | CH₂ | J64 | 0 | CH |
| 1499 | Me | Me | A21 | COOH | CH₂ | J104 | 0 | CH |
| 1500 | Me | Me | A21 | COOH | CH₂ | J105 | 0 | CH |
| 1501 | Me | Me | A21 | COOH | CH₂ | J106 | 0 | CH |
| 1502 | Me | Me | A21 | COOH | CH₂ | J107 | 0 | CH |
| 1503 | Me | Me | A21 | COOH | CH₂ | J108 | 0 | CH |
| 1504 | Me | Me | A21 | COOH | CH₂ | J109 | 0 | CH |
| 1505 | Me | Me | A21 | COOH | CH₂ | J110 | 0 | CH |
| 1506 | Me | Me | A21 | COOH | CH₂ | J111 | 0 | CH |
| 1507 | Me | Me | A21 | COOH | CH₂ | J112 | 0 | CH |
| 1508 | Me | Me | A21 | COOH | CH₂ | J113 | 0 | CH |
| 1509 | Me | Me | A21 | COOH | CH₂ | J114 | 0 | CH |
| 1510 | Cl | Cl | A22 | COOH | CH₂ | J86 | 0 | CH |
| 1511 | Cl | Cl | A22 | COOH | CH₂ | J65 | 0 | CH |
| 1512 | Cl | Cl | A22 | COOH | CH₂ | J87 | 0 | CH |
| 1513 | Cl | Cl | A22 | COOH | CH₂ | J88 | 0 | CH |
| 1514 | Cl | Cl | A22 | COOH | CH₂ | J89 | 0 | CH |
| 1515 | Cl | Cl | A22 | COOH | CH₂ | J90 | 0 | CH |

TABLE 62

| Compound No. | R₁ | R₂ | S—CH₂-A | E | G | J | m | X |
|---|---|---|---|---|---|---|---|---|
| 1516 | Cl | Cl | A22 | COOH | CH₂ | J91 | 0 | CH |
| 1517 | Cl | Cl | A22 | COOH | CH₂ | J92 | 0 | CH |
| 1518 | Cl | Cl | A22 | COOH | CH₂ | J93 | 0 | CH |
| 1519 | Cl | Cl | A22 | COOH | CH₂ | J94 | 0 | CH |
| 1520 | Cl | Cl | A22 | COOH | CH₂ | J95 | 0 | CH |
| 1521 | Cl | Cl | A22 | COOH | CH₂ | J98 | 0 | CH |
| 1522 | Cl | Cl | A22 | COOH | CH₂ | J99 | 0 | CH |
| 1523 | Cl | Cl | A22 | COOH | CH₂ | J100 | 0 | CH |
| 1524 | Cl | Cl | A22 | COOH | CH₂ | J101 | 0 | CH |
| 1525 | Cl | Cl | A22 | COOH | CH₂ | J102 | 0 | CH |
| 1526 | Cl | Cl | A22 | COOH | CH₂ | J103 | 0 | CH |
| 1527 | Cl | Cl | A22 | COOH | CH₂ | J64 | 0 | CH |
| 1528 | Cl | Cl | A22 | COOH | CH₂ | J104 | 0 | CH |
| 1529 | Cl | Cl | A22 | COOH | CH₂ | J105 | 0 | CH |
| 1530 | Cl | Cl | A22 | COOH | CH₂ | J106 | 0 | CH |
| 1531 | Cl | Cl | A22 | COOH | CH₂ | J107 | 0 | CH |
| 1532 | Cl | Cl | A22 | COOH | CH₂ | J108 | 0 | CH |
| 1533 | Cl | Cl | A22 | COOH | CH₂ | J109 | 0 | CH |
| 1534 | Cl | Cl | A22 | COOH | CH₂ | J110 | 0 | CH |
| 1535 | Cl | Cl | A22 | COOH | CH₂ | J111 | 0 | CH |
| 1536 | Cl | Cl | A22 | COOH | CH₂ | J112 | 0 | CH |
| 1537 | Cl | Cl | A22 | COOH | CH₂ | J113 | 0 | CH |
| 1538 | Cl | Cl | A22 | COOH | CH₂ | J114 | 0 | CH |
| 1539 | Cl | Cl | A20 | COOH | CH₂ | J86 | 0 | CH |
| 1540 | Cl | Cl | A20 | COOH | CH₂ | J87 | 0 | CH |
| 1541 | Cl | Cl | A20 | COOH | CH₂ | J88 | 0 | CH |
| 1542 | Cl | Cl | A20 | COOH | CH₂ | J89 | 0 | CH |
| 1543 | Cl | Cl | A20 | COOH | CH₂ | J90 | 0 | CH |
| 1544 | Cl | Cl | A20 | COOH | CH₂ | J91 | 0 | CH |
| 1545 | Cl | Cl | A20 | COOH | CH₂ | J92 | 0 | CH |
| 1546 | Cl | Cl | A20 | COOH | CH₂ | J93 | 0 | CH |
| 1547 | Cl | Cl | A20 | COOH | CH₂ | J94 | 0 | CH |
| 1548 | Cl | Cl | A20 | COOH | CH₂ | J95 | 0 | CH |
| 1549 | Cl | Cl | A20 | COOH | CH₂ | J98 | 0 | CH |
| 1550 | Cl | Cl | A20 | COOH | CH₂ | J99 | 0 | CH |
| 1551 | Cl | Cl | A20 | COOH | CH₂ | J100 | 0 | CH |
| 1552 | Cl | Cl | A20 | COOH | CH₂ | J101 | 0 | CH |
| 1553 | Cl | Cl | A20 | COOH | CH₂ | J102 | 0 | CH |
| 1554 | Cl | Cl | A20 | COOH | CH₂ | J103 | 0 | CH |
| 1556 | Cl | Cl | A20 | COOH | CH₂ | J104 | 0 | CH |
| 1557 | Cl | Cl | A20 | COOH | CH₂ | J105 | 0 | CH |
| 1558 | Cl | Cl | A20 | COOH | CH₂ | J106 | 0 | CH |
| 1559 | Cl | Cl | A20 | COOH | CH₂ | J107 | 0 | CH |
| 1560 | Cl | Cl | A20 | COOH | CH₂ | J108 | 0 | CH |
| 1561 | Cl | Cl | A20 | COOH | CH₂ | J109 | 0 | CH |
| 1562 | Cl | Cl | A20 | COOH | CH₂ | J110 | 0 | CH |
| 1563 | Cl | Cl | A20 | COOH | CH₂ | J111 | 0 | CH |
| 1564 | Cl | Cl | A20 | COOH | CH₂ | J112 | 0 | CH |
| 1565 | Cl | Cl | A20 | COOH | CH₂ | J113 | 0 | CH |

TABLE 64

| Compound No. | R₁ | R₂ | S—CH₂-A | E | G | J | m | X |
|---|---|---|---|---|---|---|---|---|
| 1566 | Cl | Cl | A20 | COOH | CH₂ | J114 | 0 | CH |
| 1567 | Cl | Cl | A21 | COOH | CH₂ | J86 | 0 | CH |
| 1568 | Cl | Cl | A21 | COOH | CH₂ | J65 | 0 | CH |
| 1569 | Cl | Cl | A21 | COOH | CH₂ | J87 | 0 | CH |
| 1570 | Cl | Cl | A21 | COOH | CH₂ | J88 | 0 | CH |
| 1571 | Cl | Cl | A21 | COOH | CH₂ | J89 | 0 | CH |
| 1572 | Cl | Cl | A21 | COOH | CH₂ | J90 | 0 | CH |
| 1573 | Cl | Cl | A21 | COOH | CH₂ | J91 | 0 | CH |
| 1574 | Cl | Cl | A21 | COOH | CH₂ | J92 | 0 | CH |
| 1575 | Cl | Cl | A21 | COOH | CH₂ | J93 | 0 | CH |
| 1576 | Cl | Cl | A21 | COOH | CH₂ | J94 | 0 | CH |
| 1577 | Cl | Cl | A21 | COOH | CH₂ | J95 | 0 | CH |
| 1578 | Cl | Cl | A21 | COOH | CH₂ | J98 | 0 | CH |
| 1579 | Cl | Cl | A21 | COOH | CH₂ | J99 | 0 | CH |
| 1580 | Cl | Cl | A21 | COOH | CH₂ | J100 | 0 | CH |
| 1581 | Cl | Cl | A21 | COOH | CH₂ | J101 | 0 | CH |
| 1582 | Cl | Cl | A21 | COOH | CH₂ | J102 | 0 | CH |
| 1583 | Cl | Cl | A21 | COOH | CH₂ | J103 | 0 | CH |
| 1584 | Cl | Cl | A21 | COOH | CH₂ | J64 | 0 | CH |
| 1585 | Cl | Cl | A21 | COOH | CH₂ | J104 | 0 | CH |
| 1586 | Cl | Cl | A21 | COOH | CH₂ | J105 | 0 | CH |
| 1587 | Cl | Cl | A21 | COOH | CH₂ | J106 | 0 | CH |
| 1588 | Cl | Cl | A21 | COOH | CH₂ | J107 | 0 | CH |
| 1589 | Cl | Cl | A21 | COOH | CH₂ | J108 | 0 | CH |
| 1590 | Cl | Cl | A21 | COOH | CH₂ | J109 | 0 | CH |

TABLE 65

| Compound No. | R₁ | R₂ | S—CH₂-A | E | G | J | m | X |
|---|---|---|---|---|---|---|---|---|
| 1591 | Cl | Cl | A21 | COOH | CH₂ | J110 | 0 | CH |
| 1592 | Cl | Cl | A21 | COOH | CH₂ | J111 | 0 | CH |
| 1593 | Cl | Cl | A21 | COOH | CH₂ | J112 | 0 | CH |
| 1594 | Cl | Cl | A21 | COOH | CH₂ | J113 | 0 | CH |
| 1595 | Cl | Cl | A21 | COOH | CH₂ | J114 | 0 | CH |
| 1596 | H | MeO | A22 | COOH | CH₂ | J86 | 0 | CH |
| 1597 | H | MeO | A22 | COOH | CH₂ | J65 | 0 | CH |
| 1598 | H | MeO | A22 | COOH | CH₂ | J87 | 0 | CH |
| 1599 | H | MeO | A22 | COOH | CH₂ | J88 | 0 | CH |
| 1600 | H | MeO | A22 | COOH | CH₂ | J89 | 0 | CH |
| 1601 | H | MeO | A22 | COOH | CH₂ | J90 | 0 | CH |
| 1602 | H | MeO | A22 | COOH | CH₂ | J91 | 0 | CH |
| 1603 | H | MeO | A22 | COOH | CH₂ | J92 | 0 | CH |
| 1604 | H | MeO | A22 | COOH | CH₂ | J93 | 0 | CH |
| 1605 | H | MeO | A22 | COOH | CH₂ | J94 | 0 | CH |
| 1606 | H | MeO | A22 | COOH | CH₂ | J95 | 0 | CH |
| 1607 | H | MeO | A22 | COOH | CH₂ | J98 | 0 | CH |
| 1608 | H | MeO | A22 | COOH | CH₂ | J99 | 0 | CH |
| 1609 | H | MeO | A22 | COOH | CH₂ | J100 | 0 | CH |
| 1610 | H | MeO | A22 | COOH | CH₂ | J101 | 0 | CH |
| 1611 | H | MeO | A22 | COOH | CH₂ | J102 | 0 | CH |
| 1612 | H | MeO | A22 | COOH | CH₂ | J103 | 0 | CH |
| 1613 | H | MeO | A22 | COOH | CH₂ | J64 | 0 | CH |
| 1614 | H | MeO | A22 | COOH | CH₂ | J104 | 0 | CH |
| 1615 | H | MeO | A22 | COOH | CH₂ | J105 | 0 | CH |

TABLE 66

| Compound No. | R₁ | R₂ | S—CH₂-A | E | G | J | m | X |
|---|---|---|---|---|---|---|---|---|
| 1616 | H | MeO | A22 | COOH | CH₂ | J106 | 0 | CH |
| 1617 | H | MeO | A22 | COOH | CH₂ | J107 | 0 | CH |
| 1618 | H | MeO | A22 | COOH | CH₂ | J108 | 0 | CH |
| 1619 | H | MeO | A22 | COOH | CH₂ | J109 | 0 | CH |
| 1620 | H | MeO | A22 | COOH | CH₂ | J110 | 0 | CH |
| 1621 | H | MeO | A22 | COOH | CH₂ | J111 | 0 | CH |
| 1622 | H | MeO | A22 | COOH | CH₂ | J112 | 0 | CH |
| 1623 | H | MeO | A22 | COOH | CH₂ | J113 | 0 | CH |
| 1624 | H | MeO | A22 | COOH | CH₂ | J114 | 0 | CH |
| 1625 | H | MeO | A20 | COOH | CH₂ | J86 | 0 | CH |
| 1626 | H | MeO | A20 | COOH | CH₂ | J65 | 0 | CH |
| 1627 | H | MeO | A20 | COOH | CH₂ | J87 | 0 | CH |
| 1628 | H | MeO | A20 | COOH | CH₂ | J88 | 0 | CH |
| 1629 | H | MeO | A20 | COOH | CH₂ | J89 | 0 | CH |
| 1630 | H | MeO | A20 | COOH | CH₂ | J90 | 0 | CH |
| 1631 | H | MeO | A20 | COOH | CH₂ | J91 | 0 | CH |
| 1632 | H | MeO | A20 | COOH | CH₂ | J92 | 0 | CH |
| 1633 | H | MeO | A20 | COOH | CH₂ | J93 | 0 | CH |
| 1634 | H | MeO | A20 | COOH | CH₂ | J94 | 0 | CH |
| 1635 | H | MeO | A20 | COOH | CH₂ | J95 | 0 | CH |
| 1636 | H | MeO | A20 | COOH | CH₂ | J98 | 0 | CH |
| 1637 | H | MeO | A20 | COOH | CH₂ | J99 | 0 | CH |
| 1638 | H | MeO | A20 | COOH | CH₂ | J100 | 0 | CH |
| 1639 | H | MeO | A20 | COOH | CH₂ | J101 | 0 | CH |
| 1640 | H | MeO | A20 | COOH | CH₂ | J102 | 0 | CH |

TABLE 67

| Compound No. | R₁ | R₂ | S—CH₂-A | E | G | J | m | X |
|---|---|---|---|---|---|---|---|---|
| 1641 | H | MeO | A20 | COOH | CH₂ | J103 | 0 | CH |
| 1642 | H | MeO | A20 | COOH | CH₂ | J64 | 0 | CH |
| 1643 | H | MeO | A20 | COOH | CH₂ | J104 | 0 | CH |
| 1644 | H | MeO | A20 | COOH | CH₂ | J105 | 0 | CH |
| 1645 | H | MeO | A20 | COOH | CH₂ | J106 | 0 | CH |
| 1646 | H | MeO | A20 | COOH | CH₂ | J107 | 0 | CH |
| 1647 | H | MeO | A20 | COOH | CH₂ | J108 | 0 | CH |
| 1648 | H | MeO | A20 | COOH | CH₂ | J109 | 0 | CH |
| 1649 | H | MeO | A20 | COOH | CH₂ | J110 | 0 | CH |
| 1650 | H | MeO | A20 | COOH | CH₂ | J111 | 0 | CH |
| 1651 | H | MeO | A20 | COOH | CH₂ | J112 | 0 | CH |
| 1652 | H | MeO | A20 | COOH | CH₂ | J113 | 0 | CH |
| 1653 | H | MeO | A20 | COOH | CH₂ | J114 | 0 | CH |
| 1654 | H | MeO | A21 | COOH | CH₂ | J86 | 0 | CH |
| 1655 | H | MeO | A21 | COOH | CH₂ | J65 | 0 | CH |
| 1656 | H | MeO | A21 | COOH | CH₂ | J87 | 0 | CH |
| 1657 | H | MeO | A21 | COOH | CH₂ | J88 | 0 | CH |
| 1658 | H | MeO | A21 | COOH | CH₂ | J89 | 0 | CH |
| 1659 | H | MeO | A21 | COOH | CH₂ | J90 | 0 | CH |
| 1660 | H | MeO | A21 | COOH | CH₂ | J91 | 0 | CH |
| 1661 | H | MeO | A21 | COOH | CH₂ | J92 | 0 | CH |
| 1662 | H | MeO | A21 | COOH | CH₂ | J93 | 0 | CH |
| 1663 | H | MeO | A21 | COOH | CH₂ | J94 | 0 | CH |
| 1664 | H | MeO | A21 | COOH | CH₂ | J95 | 0 | CH |
| 1665 | H | MeO | A21 | COOH | CH₂ | J98 | 0 | CH |

TABLE 68

| Compound No. | R₁ | R₂ | S—CH₂-A | E | G | J | m | X |
|---|---|---|---|---|---|---|---|---|
| 1666 | H | MeO | A21 | COOH | CH₂ | J99 | 0 | CH |
| 1667 | H | MeO | A21 | COOH | CH₂ | J100 | 0 | CH |
| 1668 | H | MeO | A21 | COOH | CH₂ | J101 | 0 | CH |
| 1669 | H | MeO | A21 | COOH | CH₂ | J102 | 0 | CH |
| 1670 | H | MeO | A21 | COOH | CH₂ | J103 | 0 | CH |
| 1671 | H | MeO | A21 | COOH | CH₂ | J64 | 0 | CH |
| 1672 | H | MeO | A21 | COOH | CH₂ | J104 | 0 | CH |
| 1673 | H | MeO | A21 | COOH | CH₂ | J105 | 0 | CH |
| 1674 | H | MeO | A21 | COOH | CH₂ | J106 | 0 | CH |
| 1675 | H | MeO | A21 | COOH | CH₂ | J107 | 0 | CH |
| 1676 | H | MeO | A21 | COOH | CH₂ | J108 | 0 | CH |
| 1677 | H | MeO | A21 | COOH | CH₂ | J109 | 0 | CH |
| 1678 | H | MeO | A21 | COOH | CH₂ | J110 | 0 | CH |
| 1679 | H | MeO | A21 | COOH | CH₂ | J111 | 0 | CH |
| 1680 | H | MeO | A21 | COOH | CH₂ | J112 | 0 | CH |
| 1681 | H | MeO | A21 | COOH | CH₂ | J113 | 0 | CH |
| 1682 | H | MeO | A21 | COOH | CH₂ | J114 | 0 | CH |
| 1683 | H | H | A20 | COOH | C₂H₄ | J65 | 0 | CH |
| 1684 | H | H | A20 | COOH | CH₂ | J115 | 0 | CH |
| 1685 | H | H | A20 | COOH | CH₂ | J116 | 0 | CH |

The thiobenzimidazole derivative (1) of the present invention in which E is COOH and m is 0 can be prepared by the synthetic method (A) or (B) shown below:

Synthetic Method (A) 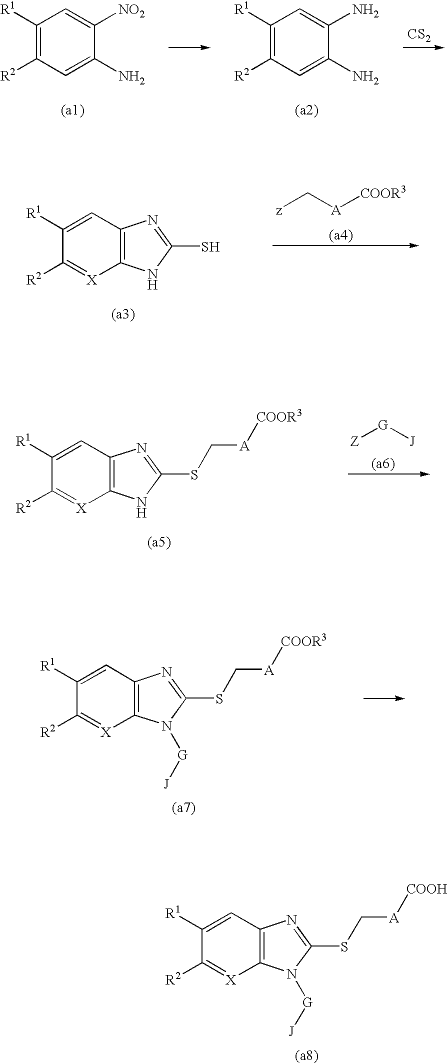
wherein Z represents a halogen, R¹, R², R³, A, G, J, and X are as defined above.

Thus, the nitro group of a 2-nitroaniline derivative (a1) is reduced to give an orthophenylene diamine (a2). CS₂ is reacted with this diamine to produce a compound (a3), with which a halide ester derivative (a4) is reacted to obtain (a5). A halide derivative (a6) is reacted therewith to obtain (a7), which is hydrolyzed to yield a benzimidazole derivative (a8) of the present invention.

The reduction of the nitro group may be carried out under a standard condition for catalytic reduction. For example, a reaction is carried out with hydrogen gas in the presence of a catalyst such as Pd—C at a temperature of room temperature to 100° C. Alternatively, a method of treatment using zinc or tin under an acidic condition, or a method of using zinc powder at a neutral or alkaline condition can be used.

The reaction of an orthophenylene diamine derivative (a2) with CS₂ may be carried out using, for example, a method as described in J. Org. Chem. 19: 631-637, 1954, or J. Med. Chem. 36: 1175-1187, 1993 (EtOH solution).

The reaction of a thiobenzimidazole (a3) and a halide ester (a4) may be carried out according to the condition of the conventional S-alkylation, for example in the presence of a base such as NaH, Et₃N, NaOH, or K₂CO₃ at a temperature of 0° C. to 200° C. under stirring.

The reaction of a thiobenzimidazole (a5) and a halide derivative (a6) may be carried out according to the condition for the conventional N-alkylation or N-acylation, for example in the presence of a base such as NaH, Et₃N, NaOH, or K₂CO₃ at a temperature of 0° C. to 200°C. under stirring.

As the elimination reaction of the carboxy protecting group R³, preferably a method of hydrolysis is employed using an alkali such as lithium hydroxide or an acid such as trifluoroacetic acid.

Synthetic Method (B)

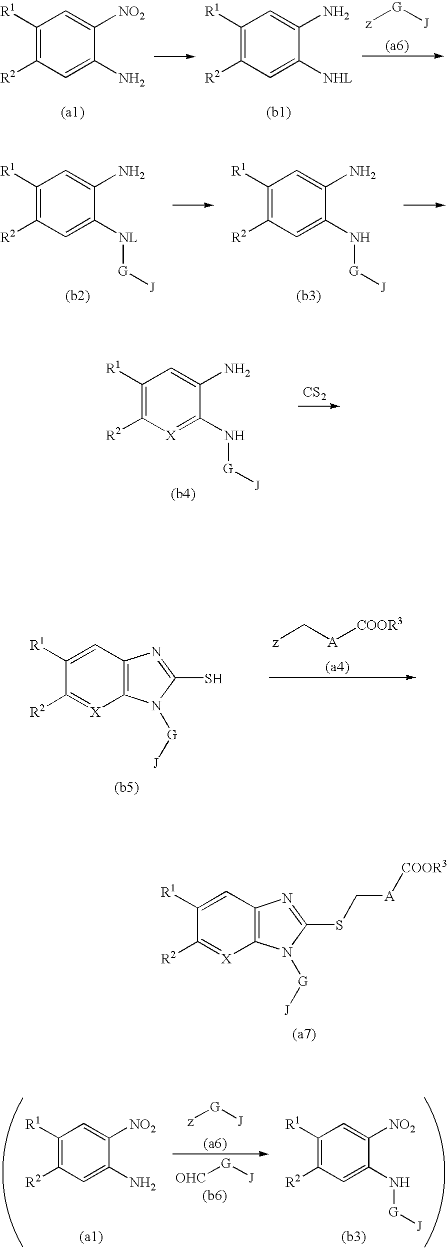

Thus, the amino group of a 2-nitroaniline derivative (a1) can be protected with L to give (b1). A halide derivative (a6) is reacted therewith to obtain (b2), from which L is deprotected to obtain (b3). The nitro group of (b3) is reduced to obtain an orthophenylene diamine derivative (b4). CS₂ is reacted therewith to yield a compound (b5), with which a halide ester derivative (a4) is reacted to obtain (a7) which may be hydrolyzed to yield a benzimidazole derivative of the present invention. Alternatively, it is also possible to obtain a compound (b3) directly by allowing the 2-nitroaniline derivative (a1) as it is unprotected to be reacted to a halide derivative (a6) or an aldehyde derivative (b6). As the protecting group L, there can be mentioned a trifluoroacetic acetyl group, an acetyl group, a t-butoxycarbonyl group, a benzyl group, and the like. The reaction of the 2-nitroaniline derivative (a1) and the aldehyde derivative (b6) may be carried out according to the conditions of the conventional reductive amination using a reducing agent such as a complex hydrogen compound, for example LiAlH₄, NaBH₄, NaB₃CN, NaBH(OAc)₃, etc. or diborane, in a solvent such as ethanol, methanol, and dichloromethane at a temperature condition of 0° C. to 200° C. The other reactions may be carried out as in the Synthetic method (A).

The thiobenzimidazole derivative (1) of the present invention in which E is COOH, m is 0, and G is an amide bond can be prepared by the synthetic method (C) shown below:

Synthetic Method (C) 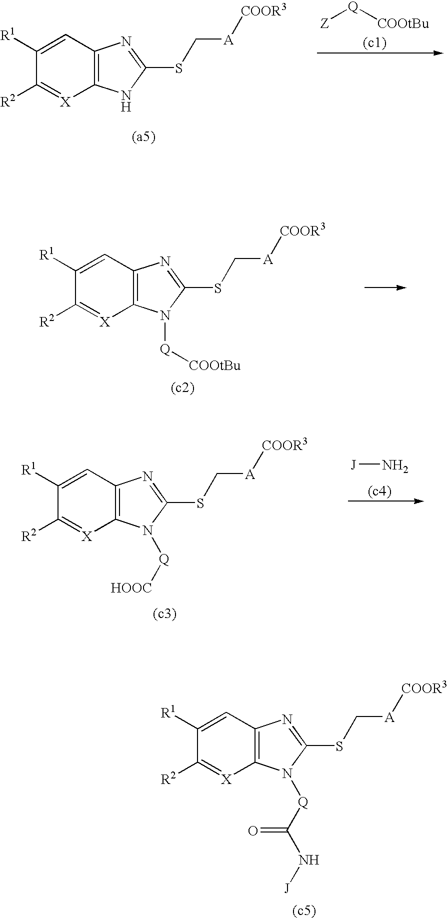
wherein Q represents a methylene group, a phenylene group, etc., and Z represents a halogen. R¹, R², R³, A, J, and X are as defined above, provided that R³ is a protecting group such as an ethyl group, a methyl group, etc. inactive in an acid.

Thus, a tert-butyl ester halide derivative (c1) is reacted with a thiobenzimidazole compound (a5) to obtain a compound (c2), which is subjected to hydrolysis under an acidic condition to yield (c3). An amine derivative (c4) is reacted therewith to yield (c5), which is subjected to hydrolysis to obtain the benzimidazole derivative of the present invention.

The condensation amidation may be carried out by a conventional method using a condensing agent. As the condensing agent, there can be mentioned DCC, DIPC, EDC=WSCI, WSCI.HCl, BOP, DPPA, etc., which may be used alone or in combination with HONSu, HOBt, HOOBt, etc. The reaction may be carried out in a appropriate solvent such as THF, chloroform, t-butanol, etc. at a temperature condition of 0° C. to 200° C. The other reactions may be carried out as in the Synthetic method (A).

The thiobenzimidazole derivative (1) of the present invention in which E is COOH, m is 0, and G is an ether bond can be prepared by the synthetic method (D) shown below:

Synthetic Method (D) 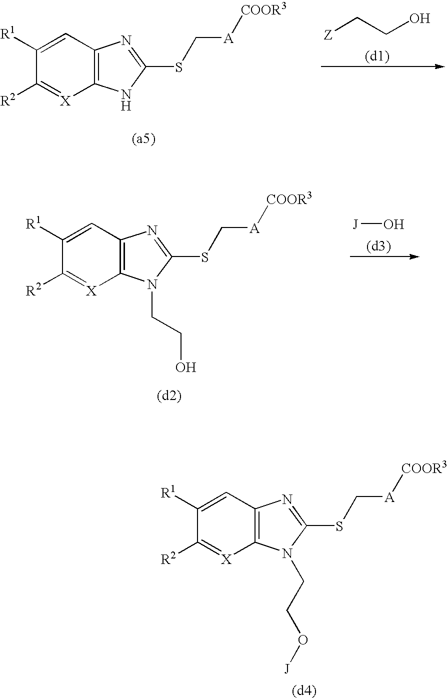
wherein Z represents a halogen, R¹, R², R³, A, J, and X are as defined above.

Thus, a thiobenzimidazole compound (a5) is reacted with, for example, a halide alcohol derivative (d1) to yield a compound (d2). A phenol derivative (d3) is reacted therewith to yield an ether (d4), which is subjected to hydrolysis to yield a benzimidazole derivative (a8) of the present invention.

The etherification may be carried out using a phosphine compound such as triphenyl phosphine and tributyl phosphine and an azo compound such as DEAD and TMAD in a suitable solvent such as N-methylmorpholine and THF at a temperature of 0° C. to 200° C. in a Mitsunobu reaction or a related reaction thereof. The other reactions may be carried out as in the Synthetic method (A).

The thiobenzimidazole derivative (1) of the present invention in which E is a tetrazole and m is 0 can be prepared by the synthetic method (E) shown below:

Synthetic Method (E) 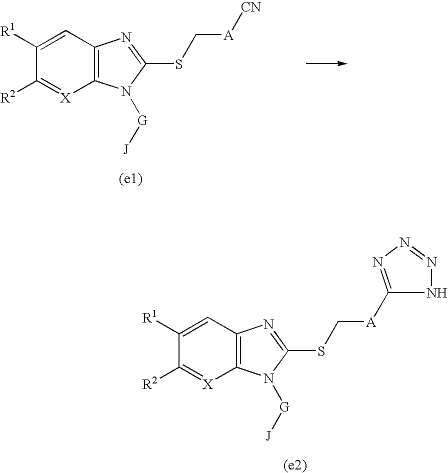
wherein R¹, R², A, G, J, and X are as defined above.

A nitrile (e1) is reacted with various azi compounds to be converted to a tetrazole (e2).

As the azi compound, there can be mentioned a trialkyltin azide compound such as trimethyltin azide, and hydrazoic acid or an ammonium salt thereof. When an organic tin azide compound is used, 1-4 fold molar amount is used relative to the compound (e1). When hydrazoic acid or an ammonium salt thereof is used, 1-5 fold molar amount of sodium azide or a tertiary amine such as ammonium chloride and triethylamine may be used relative to the compound (e1). Each reaction may be carried out at at temperature of 0° C. to 200° C. in a solvent such as toluene, benzene and DMF.

The thiobenzimidazole derivative (1) of the present invention in which m is 1 or 2 can be prepared by the synthetic method (F) shown below.

Synthetic Method (F) 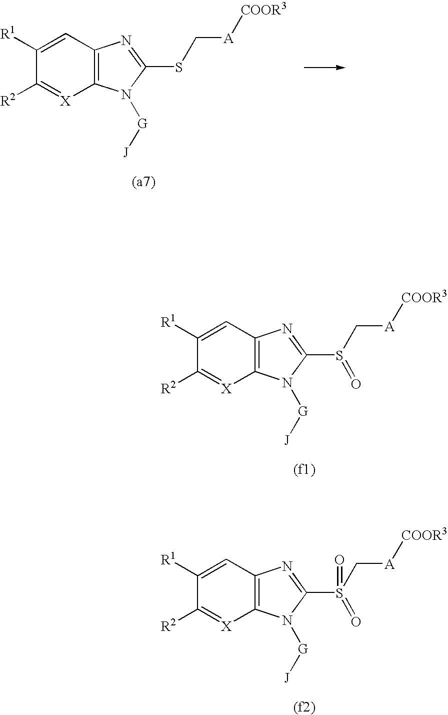
wherein R¹, R², R³, A, G, J, and X are as defined above.

Thus, a thiobenzimidazole compound (a7) may be reacted with a peroxide compound in a suitable medium to yield a sulfoxide derivative (f1) and/or a sulfone derivative ([2). As the peroxide compound used, there can be mentioned perbenzoic acid, m-chloroperbenzoic acid, peracetic acid, hydrogeny peroxide, and the like, and as the solvent used, there can be mentioned chloroform, dichloromethane, and the like. The ratio of the compound (a7) to the peroxide compound used is selected from, but not limited to, a broad range as appropriate, and generally 1.2 to 5 fold molar amount, for example, may be preferably used. Each reaction is carried out generally at about 0 to 50° C., and preferably at 0° C. to room temperature, and is generally complete in about 4-20 hours.

Benzimidazole derivative (1) of the present invention can be produced according to the following Synthesis Method (G) in the case M is a single bond:

Synthetic Method (G) 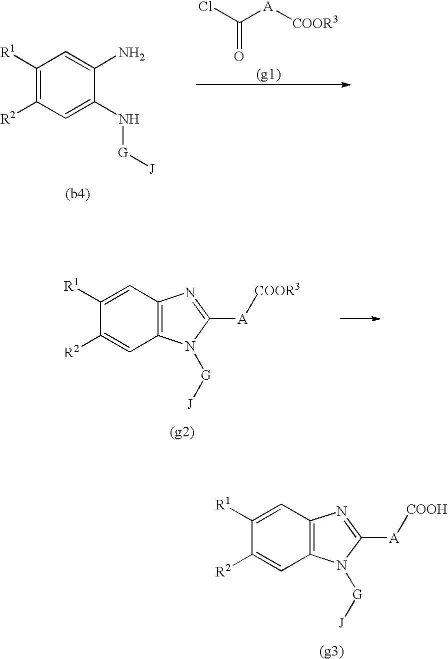
wherein, X, A, G, J and R³ are as defined above.

Namely, benzimidazole derivative (g2) of the present invention can be obtained by reacting a known acid chloride derivative (g1) with a diamine compound (b4). In addition, hydrolyzing —COOR³ of (g2) as necessary allows the obtaining of benzimidazole derivative (g3) in which R³ is a hydrogen atom.

Furthermore, the cyclization reaction is described in the Journal of Medical Chemistry (J. Med. Chem.), 1993, Vol. 36, pages 1175-1187.

In addition, Z-G-J described in synthesis methods (A) through (F) can be synthesized by referring to a large number of publications.

For example, a benzothiophene halide derivative can be synthesized by referring to the following literature and patent specification. 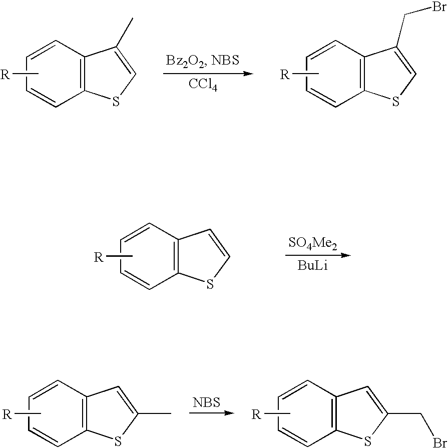
J. Chem. Soc. (1965), 774
J. Chem. Soc. Perkin Trans 1, (1972), 3011
JACS, 74, 664, (1951); U.S. Pat. No. 4,282,227

These compounds can also be synthesized by referring to the following literature and patent specifications. Namely, these compounds can be synthesized not only by the reactions described in the following literature, but also by combining typical reactions such as oxidation-reduction or OH halogenation.

J Chem Soc, (1965), 774; Bull Chem Soc Jpn (1968), 41, 2215; Japanese Unexamined Patent Publication No. 10-298180; Sulfur Reports, (1999), Vol. 22, 1-47; J Chem Soc comm., (1988), 888: J. Heterocyclic Chem., 19, 859, (1982); Synthetic Communication, (1991), 21, 959; Tetrahedron Letters, (1992), Vol. 33, No. 49, 7499; Synthetic Communications, (1993), 23(6), 743; Japanese Unexamined Patent Publication No. 2000-239270; J. Med. Chem., (1985), 28, 1896; Arch Pharm, (1975), 308, 7, 513; Khim Gerotsikl Soedin, (1973), 8, 1026; Bull. Chem. Soc. Jpn., (1997), 70, 891; J. Chem. Soc. Perkin1, (1973), 750; J. Chem. Soc. Chem. Comm., (1974), 849; J. Chem. Soc. Comm. (1972), 83

In particular, the hydroxymethyl form at position 3 of benzothiophene can be synthesized easily by referring to J. Chem. Soc. Chem. Comm., (1974), 849.

With respect to iodides, the Cl and Br forms can be obtained by halogen exchange with NaI and so forth.

In addition, the quaternary ammonium salt derivative of benzothiophene can be synthesized by reacting a suitable amine such as dimethylamine with the previously mentioned benzothiophene halide derivative. In addition, it may also be synthesized in the following manner:

Synthetic Method (H) 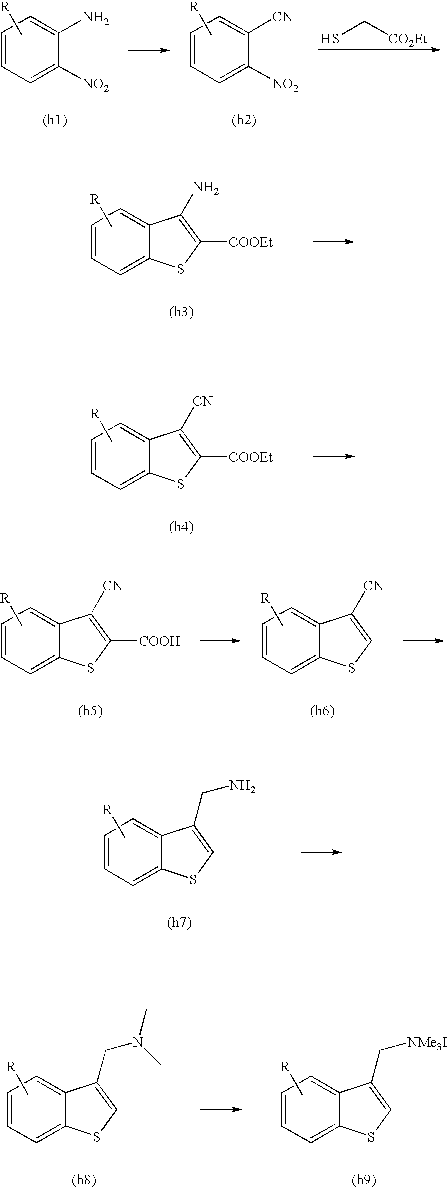
wherein, R represents one or more substituents in the above-mentioned J, the number of substituents is optional, and the substituents may be independent substituents.

Namely, cyclic benzothiophene derivative (h3) is obtained by converting the amino group of 2-nitroaniline derivative (h1) to a cyano form (h2) and reacting with ethyl 2-mercaptoacetate. Moreover, carboxylic acid (h5) is obtained by cyanating the amino group to a cyano form (h4) followed by ester hydrolysis. The carboxylic acid is then decarboxylated to obtain (h6). Continuing, the cyano group is reduced to convert to an amino form (h7) followed by N-dimethylation to obtain (h8) and then followed by N-methylation to be able to obtain quaternary salt (h9).

Cyanation of the amino group of 2-nitroaniline derivative (h1) by converting the amino group to diazonium using, for example, hydrochloric acid or sodium sulfite, and then further reacting with copper (I) chloride and potassium cyanide to convert to the cyano form.

Reaction from cyano form (h2) to benzothiophene derivative (h3) can be carried out to obtain cyclic benzothiophene derivative (h3) by heating with ethyl 2-mercaptoacetate in a suitable solvent such as DMF in the presence of a suitable basic reagent by referring to the method described in, for example, Synthetic Communications, 23(6), 743-748 (1993); or Farmaco, Ed. Sci., 43, 1165 (1988).

With respect to the cyanation of (h3), (h3) can be converted to the cyano form (h4) by reacting copper cyanide and t-butyl sulfite in a suitable solvent such as DMSO under suitable temperature conditions.

Ester hydrolysis can be carried out by routinely used methods. For example, carboxylic acid (h5) can be obtained by ester hydrolysis in a suitable solvent such as THF-MeOH in the presence of a suitable basic reagent such as sodium hydroxide.

The carboxylic acid decarboxylation reaction can be carried out by heating in a suitable solvent such as quinoline solvent in the presence of a copper catalyst.

Reduction of the cyano group to an amino group can be carried out to obtain the amino form by, for example, reducing in a suitable solvent such as Et₂O-THF under suitable temperature conditions using a suitable reducing agent such as lithium aluminum hydride.

Methylation of the amino group can be carried out by heating in, for example, formic acid or aqueous formalin solution.

Conversion of the amino group to a quaternary salt can be carried out by, for example, reacting with methyl iodide in ethanol solvent.

Indole quaternary amine salt derivative can be synthesized according to, for example, the following method:

Synthetic Method (K) 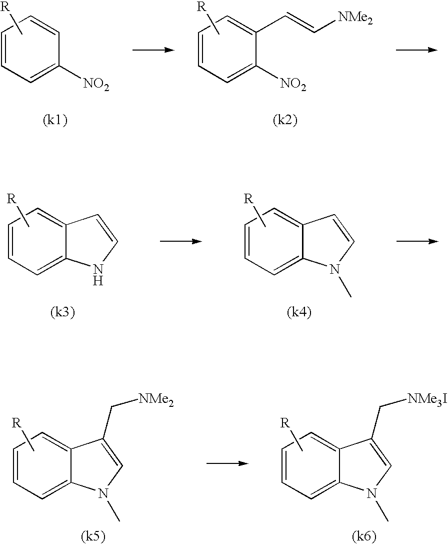
wherein, R represents one or more substituents in the above-mentioned J, the number of substituents is optional, and the substituents may be independent substituents.

Namely, nitro form (k1) is converted to an enamine (k2) by enanimation followed by converting to the indole form (k3) by indole cyclization according to the method of Reissert. Moreover, the 3^{rd} position dimethylaminomethyl form (k5) is obtained according to the Mannich reaction following N-dimethylation and this is followed by N-methylation to be able to obtain the quaternary amine salt (k6).

The enamination reaction can be carried out by, for example, heating the O-nitrotoluene derivative (k1) with N,N-dimethylformamide dimethylacetal and pyrrolidine in a suitable solvent such as DMF.

The indole cyclization reaction can be carried out by reacting at room temperature using hydrogen gas in the presence of Raney nickel in a suitable solvent such as toluene.

N-methylation can be carried out by, for example, heating in DMF solvent using t-butoxypotassium or dimethyl oxalate.

3^{rd} position dimethylaminomethylation can be carried out by, for example, using the Mannich reaction and reacting at room temperature in dioxane-acetic acid solvent using aqueous formalin solution or aqueous dimethylamine solution.

In addition, the indole derivative can be synthesized by referring to the literature of Heterocycles, Vol. 22, No. 1, 195, (1984).

Moreover, benzothiophene, indole and other heterocyclic halides and quaternary salts can be synthesized by referring to other references in the literature such as Heterocyclic Compound Chemistry, (Kondansha Scientific, H. Yamanaka, ed.).

The benzimidazole derivatives of the present invention can be converted, as needed, to medically acceptable non-toxic cation salts. As such a salt, there can be mentioned an alkali metal ion such as Na⁺ and K⁺; an alkaline earth metal ion such as Mg²⁺ and Ca⁺; a metal ion such as Al³⁺ and Zn²⁺; or an organic base such as ammonia, triethylamine, ethylenediamine, propanediamine, pyrrolidine, piperidine, piperadine, pyridine, lysine, choline, ethanolamine, N,N-diethylethanolamine, 4-hydroxypiperidine, glucosamine, and N-methylglucamine. Among them, Na⁺, Ca²⁺, lysine, choline, N,N-dimethylethanolamine and N-methylglucamine are preferred.

The benzimidazole derivatives of the present invention inhibit human chymase activity. Specifically, their IC50 is not greater than 1000, preferably not smaller than 0.01 and less than 1000, and more preferably not smaller than 0.05 and less than 500. The benzimidazole derivatives of the present invention having such excellent inhibitory action on human chymase can be used as clinically applicable preventive and/or therapeutic agents for various diseases.

The benzimidazole derivatives of the present invention can be administered as pharmaceutical compositions together with pharmaceutically acceptable carriers by oral or parenteral routes after being shaped into various dosage forms. As the parenteral administration, there can be mentioned intravenous, subcutaneous, intramuscular, percutaneous, rectal, nasal, and eye drop administration.

Dosage forms for said pharmaceutical compositions include the following. For example, in the case of oral administration, there can be mentioned dosage forms such as tablets, pills, granules, powders, solutions, suspensions, syrups, and capsules.

As used herein, tablets are shaped by a conventional method using a pharmaceutically acceptable carrier such as an excipient, a binder, and a disintegrant. Pills, granules, and powders can also be shaped by a conventional method using an excipient etc. Solutions, suspensions, and syrups may be shaped by a conventional method using glycerin esters, alcohols, water, vegetable oils, and the like. Capsules can be shaped by filling a granule, a powder, and a solution into a capsule made of gelatin etc.

Among the parenteral preparations, those for intravenous, subcutaneous, and intramuscular administration can be administered as an injection. As injections, a benzoic acid derivative is dissolved in a water soluble liquid such as physiological saline, or in a non-water soluble liquid comprising an organic ester such as propylene glycol, polyethylene glycol, and a vegetable oil.

In the case of percutaneous administration, dosage forms such as ointments and creams can be used. Ointments can be prepared by mixing a benzoic acid derivative with a fat or lipid, vaseline, etc., and creams can be prepared by mixing a benzoic acid derivative with an emulsifier.

In the case of rectal administration, gelatin soft capsules can be used to prepare suppositories.

In the case of nasal administration, they can be used as a formulation comprising a liquid or powder composition. As the base for liquid formulations, water, saline, a phosphate buffer, an acetate buffer etc. can be used, and furthermore they may include a surfactant, an antioxidant, a stabilizer, a preservative, and a thickening agent. As the base for powder formulations, there can be mentioned polyacrylic acid salts that are readily solubule in water, cellulose lower alkyl ethers, polyethylene glycol, polyvinylpyrrolidone, amylose, pullulan, etc. that are water-absorptive, or celluloses, starches, proteins, gums, crosslinked vinyl polymers, etc. that are hardly water-soluble, and preferably they are water-absorptive. Alternatively, they may be combined. Furthermore, for powder formulations, an antioxidant, a colorant, a preservative, a disinfectant, a corrigent, etc. can be added. Such liquid formulations and powder formulations can be administered using, for example, a spraying device etc.

For eye drop administration, they can be used as aqueous or non-aqueous eye drops. For the aqueous eye drops, sterile purified water, physiological saline etc. can be used as a solvent. When sterile purified water is used as the solvent, a suspending agent such as a surfactant and a polymer thickener may be added to prepare an aqueous eye drop suspension. Alternatively, a solubilizing agent such as a nonionic surfactant may be added to prepare a soluble eye drop solution. The non-aqueous eye drop can use a non-aqueous solvent for injection as a solvent, and can be used as a non-aqueous eye drop solution.

In the case where administration to the eye is performed by a method other than the eye drop, dosage forms such as an eye ointment, an application solution, an epipastic, and an insert can be used.

In the case of nasal or oral inhalation, they are inhaled as a solution or a suspension of the benzimidazole derivatives of the present invention with a commonly used pharmaceutical excipient using, for example, an aerosol spray for inhalation, etc. Alternatively, the benzimidazole derivatives of the present invention in a lyophilized powder form can be administered to the lung using an inhaling device that permits direct contact to the lung.

To such various formulations, pharmaceutically acceptable carriers such as an isotonic agent, a preservative, a disinfectant, a wetting agent, a buffering agent, an emulsifier, a dispersant, a stabilizer, etc. can be added as needed.

To these formulations, blending of an antimicrobial agent, a treatment such as filtration through a bacteria-retaining filter, heating, radiation, etc. can be carried out for sterilization. Alternatively, sterile solid formulations can be prepared, which may be used by dissolving or suspending them in an appropriate sterile solution immediately prior to use.

The dosages of the benzimidazole derivatives of the present invention vary depending on the type of diseases, route of administration, the condition, age, sex, body weight etc. of the patient, but they are generally in the range of about 1 to 500 mg/day/patient for oral administration, and preferably 1 to 300 mg/day/patient. In the case of parenteral administration such as intravenous, subcutaneous, intramuscular, percutaneous, rectal, nasal, eye drop, and inhalation administration, they are about 0.1 to 100 mg/day/patient, and preferably 0.3 to 30 mg/day/patient.

When the benzimidazole derivatives of the present invention are used as a preventive agent, they can be administered according to a known method depending on each condition.

As the target diseases for the preventive and/or therapeutic agents of the present invention, there can be mentioned, for example, diseases of respiratory organs such as bronchial asthma, inflammatory/allergic diseases such as allergic rhinitis, atopic dermatitis, and urticaria; diseases of circulatory organs such as sclerosing vascular lesions, intravascular stenosis, disturbances of peripheral circulation, renal failure, and cardiac failure; diseases of bone/cartilage metabolism such as rheumatoid arthritis and osteoarthritis.

### EXAMPLES

The present invention will now be explained in more detail with reference to Preparation Examples, Working Examples, and Test Examples. It should be noted, however, that these examples do not limit the scope of the invention in any way.

### Reference Example 1

### Preparation of 5,6-dimethylbenzimidazole-2-thiol

To 5,6-dimethylorthophenylene diamine (4.5 g, 33 mmol) in pyridine (40 ml) was added carbon disulfide (40 ml, 0.66 mol). The resulting solution was heated to reflux under stirring for 18 hours, to which was added water, followed by extraction with ethyl acetate. After drying the ethyl acetate phase with anhydrous magnesium sulfate, it was concentrated, and dried under reduced pressure at 80° C. for 6 hours to obtain the title compound (4.1 g, yield 70%).

¹H-NMR (270 Mhz, DMSO-d6) (ppm): 12.30 (br, 1H), 6.91 (s, 2H), 2.21 (s, 6H)

### Reference Example 2

### Preparation of 2-((5,6-dimethylbenzimidazole-2-ylthio)methyl)benzoic acid methyl ester

To the resulting 5,6-dimethylbenzimidazole-2-thiol (89 mg, 0.50 mmol) in dimethylformamide (2 ml), triethylamine (84 μl, 0.6 mmol) and 2-bromomethyl benzoic acid methyl ester (137 mg, 0.6 mmol) were added. After the resulting solution was stirred at 80° C. for 1.5 hours, water was added, followed by extraction with ethyl acetate. After drying the ethyl acetate phase with anhydrous magnesium sulfate, it was concentrated, and the residue was purified by silica gel column chromatography (hexane:ethyl acetate=3:1) to obtain the title compound (146 mg, yield 90%). The compound was confirmed by identification of molecular weight using LC-MS.

Calculated M=326.11, measured (M+H)⁺=327.2

### Reference Example 3

In a similar manner to Reference Example 2, the following compounds were synthesized. The compounds were confirmed by identification of molecular weight using LC-MS.
3-((5,6-dimethylbenzimidazole-2-ylthio)methyl)pyridine-2-carboxylic acid ethyl ester

Calculated M=341.12, found (M+H)⁺=342.2
2-((5,6-dimethylbenzimidazole-2-ylthio)methyl)furane-3-carboxylic acid methyl ester

Calculated M=316.09, found (M+H)⁺=317.2
3-((5,6-dimethylbenzimidazole-2-ylthio)methyl)thiphene-2-carboxylic acid methyl ester

Calculated M=332.07, found (M+H)⁺=333.2
2-(benzimidazole-2-ylthiomethyl)benzoic acid methyl ester

Calculated M=298.08, found (M+H)⁺=299.2
3-(benzimidazole-2-ylthiomethyl)pyridine-2-carboxylic acid ethyl ester

Calculated M=313.09, found (M+H)⁺=314.2
3-(benzimidazole-2-ylthiomethyl)thiophene-2-carboxylic acid methyl ester

Calculated M=304.03, found (M+H)⁺=305.2
2-(benzimidazole-2-ylthiomethyl)furane-3-carboxylic acid methyl ester

Calculated M=288.06, found (M+H)⁺=289.2
4-benzimidazole-2-ylthiobutanoic acid methyl ester

Calculated M=264.09, found (M+H)⁺=265.2
2-((5,6-dichlorobenzimidazole-2-ylthio)methyl)-5-chlorobenzoic acid methyl ester

Calculated M=399.96, found (M+H)⁺=401.2
2-(benzimidazole-2-ylthiomethyl)-5-chlorobenzoic acid methyl ester

Calculated M=332.04, found (M+H)⁺=333.2
4-((5,6-dimethylbenzimidazole-2-ylthio)butanoic acid ethyl ester

Calculated M=292.12, found (M+H)⁺=293.40
2-((5,6-dichlorobenzimidazole-2-ylthio)methyl)-benzoic acid methyl ester

Calculated M=366.00, found (M+H)⁺=367.0
2-((5,6-dichlorobenzimidazole-2-ylthio)methyl)pyridine-3-carboxylic acid methyl ester

Calculated M=366.99, found (M+H)⁺=368.0

### Example 1

### Preparation of Compound No. 143

Sodium hydride (11 mg, 0.306 mmol) and 2 ml of tetrahydrofuran was added to a previously dried reaction vessel. To the mixture were added 2-((5,6-dimethylbenzimidazole-2-ylthio)methyl)benzoic acid methyl ester (50 mg, 0.153 mmol) and 1-chloromethylnaphthalene (69 μl, 0.459 mmol), which was then stirred at 60° C. for 45 minutes. Water was added thereto, followed by extraction with ethyl acetate. After drying the ethyl acetate phase with anhydrous sodium sulfate, it was concentrated, and the residue was purified by silica gel column chromatography (hexane:ethyl acetate=4:1) to obtain 2-((5,6-dimethyl-1-(1-naphthylmethyl)benzimidazole-2-ylthio)methyl)benzoic acid methyl ester (yield 32%).

To 2-((5,6-dimethyl-1-(1-naphthylmethyl)benzimidazole-2-ylthio)methyl)benzoic acid methyl ester (23 mg, 0.08 mmol) in tetrahydrofuran (1 ml) and methanol (0.5 ml), 4N aqueous sodium hydroxide solution (0.25 ml) was added. After stirring at room temperature for 5 hours, 6N hydrochloric acid was added to stop the reaction, followed by extraction with ethyl acetate. The ethyl acetate phase was washed with saturated saline, and then dried in anhydrous sodium sulfate. The solvent was evaporated under reduced pressure to obtain the title compound (24 mg, yield quantitative).

The compound was confirmed by identification of molecular weight using LC-MS.

Calculated M=452.16, found (M+H)⁺=453.2

### Example 2

In a similar manner to Working Example 1, the compounds in Tables 69 to 73 were synthesized using the compounds in Reference Examples 2 or 3 and various halide derivatives. The compounds were confirmed by identification of molecular weight using LC-MS.

TABLE 69

| | | | Recovery % |
|---|---|---|---|
| Compound No. | Calculated M | Found (M + H)⁺ | (overall) |
| 390 | 406.14 | 407.2 | 29 |
| 391 | 422.11 | 423.2 | 16 |
| 315 | 417.15 | 418.2 | 32 |
| 376 | 406.14 | 407.2 | 25 |
| 333 | 417.15 | 418.2 | 6 |
| 82 | 416.16 | 417.2 | 12 |
| 83 | 416.16 | 417.2 | 9 |
| 84 | 416.16 | 417.2 | 33 |
| 97 | 432.15 | 433.2 | 18 |
| 98 | 432.15 | 433.2 | 26 |
| 99 | 432.15 | 433.2 | 8 |
| 94 | 470.13 | 471.2 | 14 |
| 95 | 470.13 | 471.2 | 10 |
| 96 | 470.13 | 471.2 | 13 |
| 100 | 486.12 | 487.2 | 26 |
| 101 | 486.12 | 487.2 | 8 |
| 85 | 420.13 | 421.2 | 9 |
| 86 | 420.13 | 421.0 | 12 |
| 87 | 420.13 | 421.2 | 44 |
| 88 | 436.10 | 437.2 | 42 |
| 89 | 436.10 | 437.2 | 40 |
| 90 | 436.10 | 437.2 | 28 |
| 91 | 480.07 | 481.0 | 12 |
| 103 | 427.14 | 428.2 | 12 |
| 104 | 427.14 | 428.2 | 6 |
| 105 | 427.14 | 428.2 | 11 |
| 784 | 434.11 | 435.2 | 36 |

TABLE 70

| | | | Recovery % |
|---|---|---|---|
| Compound No. | Calculated M | Found (M + H)⁺ | (overall) |
| 787 | 468.07 | 469.2 | 31 |
| 112 | 418.14 | 419.2 | 40 |
| 141 | 480.12 | 481.0 | 72 |
| 138 | 494.17 | 495.2 | 34 |
| 135 | 446.13 | 447.2 | 19 |
| 137 | 478.17 | 479.2 | 6 |
| 143 | 452.16 | 453.2 | 35 |
| 142 | 452.16 | 453.0 | 30 |
| 139 | 428.16 | 429.4 | 22 |
| 140 | 458.20 | 459.2 | 5 |
| 63 | 424.12 | 425.2 | 25 |
| 311 | 453.15 | 454.5 | 21 |
| 115 | 430.17 | 431.5 | 68 |
| 116 | 430.17 | 431.5 | 52 |
| 117 | 430.17 | 431.5 | 41 |
| 118 | 430.17 | 431.5 | 56 |
| 125 | 462.16 | 463.0 | 59 |
| 126 | 462.16 | 463.0 | 25 |
| 128 | 492.17 | 493.0 | 27 |
| 134 | 446.13 | 447.0 | 34 |
| 108 | 446.17 | 447.0 | 75 |
| 107 | 446.17 | 447.0 | 57 |
| 119 | 470.06 | 471.0 | 36 |
| 120 | 470.06 | 471.0 | 57 |
| 121 | 470.06 | 471.0 | 60 |
| 122 | 470.06 | 471.0 | 37 |
| 123 | 430.17 | 431.3 | 57 |

TABLE 71

| | | | Recovery % |
|---|---|---|---|
| Compound No. | Calculated M | Found (M + H)⁺ | (overall) |
| 124 | 462.16 | 463.3 | 67 |
| 127 | 462.16 | 463.3 | 62 |
| 129 | 446.17 | 447.3 | 47 |
| 130 | 446.17 | 447.3 | 40 |
| 319 | 425.12 | 426.3 | 30 |
| 506 | 466.17 | 467.2 | 16 |
| 505 | 466.17 | 467.0 | 14 |
| 93 | 480.07 | 481.0 | 45 |
| 136 | 478.17 | 479.2 | 60 |
| 37 | 402.14 | 403.4 | 25 |
| 39 | 442.03 | 443.0 | 51 |
| 317 | 403.14 | 404.0 | 56 |
| 318 | 443.03 | 444.0 | 46 |
| 380 | 442.14 | 443.2 | 51 |
| 377 | 420.15 | 421.2 | 34 |
| 378 | 460.04 | 461.0 | 30 |
| 386 | 414.10 | 415.2 | 37 |
| 383 | 392.12 | 393.2 | 30 |
| 384 | 432.01 | 433.0 | 29 |
| 395 | 458.11 | 459.2 | 23 |
| 392 | 436.13 | 437.2 | 15 |
| 393 | 476.02 | 477.0 | 15 |
| 401 | 430.08 | 431.2 | 50 |
| 398 | 408.10 | 409.2 | 20 |
| 399 | 447.99 | 449.0 | 7 |

TABLE 72

| | | | Recovery % |
|---|---|---|---|
| Compound No. | Calculated M | Found (M + H)⁺ | (overall) |
| 544 | 476.18 | 377.2 | 62 |
| 50 | 418.14 | 419.2 | 42 |
| 459 | 382.08 | 383.2 | 65 |
| 402 | 436.04 | 437.2 | 50 |
| 1 | 388.12 | 389.0 | 38 |
| 161 | 456.05 | 457.0 | 54 |
| 81 | 402.14 | 403.3 | 57 |
| 154 | 444.13 | 445.0 | 32 |
| 160 | 408.10 | 409.0 | 72 |
| 159 | 421.15 | 422.2 | 84 |
| 148 | 482.17 | 483.5 | 64 |
| 149 | 453.15 | 454.5 | 71 |
| 155 | 459.11 | 460.0 | 64 |
| 150 | 453.15 | 454.2 | 36 |
| 151 | 487.11 | 488.1 | 62 |
| 153 | 460.10 | 461.0 | 69 |
| 152 | 454.15 | 455.0 | 62 |
| 64 | 430.08 | 431.2 | 85 |
| 455 | 410.11 | 411.2 | 17 |
| 596 | 430.14 | 431.2 | 56 |
| 539 | 418.17 | 419.2 | 20 |
| 349 | 436.10 | 437.1 | 50 |
| 352 | 458.09 | 459.2 | 74 |
| 168 | 470.06 | 471.1 | 57 |
| 355 | 504.02 | 505.0 | 26 |
| 174 | 492.05 | 493.0 | 89 |
| 358 | 526.01 | 527.1 | 38 |

TABLE 73

| | | | Recovery % |
|---|---|---|---|
| Compound No. | Calculated M | Found (M + H)⁺ | (overall) |
| 324 | 493.04 | 494.2 | 32 |
| 320 | 431.08 | 432.1 | 15 |
| 147 | 466.17 | 467.2 | 72 |
| 616 | 490.16 | 491.2 | 22 |
| 805 | 382.17 | 383.2 | 52 |
| 804 | 368.16 | 369.2 | 56 |
| 66 | 438.14 | 440.2 | 54 |
| 592 | 430.14 | 432.3 | 5 |
| 811 | 380.16 | 382.2 | 72 |
| 582 | 436.06 | 437.1 | 59 |
| 580 | 436.06 | 437.1 | 59 |
| 584 | 480.03 | 483.1 | 37 |
| 583 | 480.03 | 483.0 | 52 |
| 578 | 420.09 | 421.2 | 30 |
| 574 | 416.12 | 417.2 | 39 |
| 595 | 452.12 | 453.2 | 22 |
| 594 | 478.14 | 479.1 | 23 |
| 588 | 432.11 | 433.1 | 65 |
| 587 | 432.11 | 433.2 | 48 |
| 586 | 432.11 | 433.1 | 50 |
| 590 | 427.10 | 428.2 | 24 |
| 589 | 427.10 | 428.3 | 17 |

### Example 3

### Preparation of Compound No. 547

Triethylamine (276 μl, 1.98 mmol) and 2-(bromoethyl)benzoic acid t-butyl ester (538 mg, 1.99 mmol) were added to 5,6-dimethylbenzimidazole-2-thiol (236 mg, 1.32 mmol) in 2 ml of dimethylformamide, which was then stirred at 80° C. for 3 hours. After the reaction was complete, water was added, followed by extraction with ethyl acetate. After drying the ethyl acetate phase with anhydrous sodium sulfate, it was concentrated, and the residue was purified by silica gel column chromatography (hexane:ethyl acetate=3:1) to obtain 2-((5,6-dimethylbenzimidazole-2-ylthio)methyl)benzoic acid t-butyl ester (288 mg, yield 59%).

2-((5,6-dimethylbenzimidazole-2-ylthio)methyl)benzoic acid t-butyl ester (30 mg, 0.082 mmol) was dissolved in 3 ml of chloroform, to which triethylamine (17 μl, 0.123 mmol) and benzoyl chloride (14 μl, 0.123 mmol) were sequentially added and the mixture was stirred at room temperature for 2 hours. After the reaction was complete, water was added, followed by extraction with ethyl acetate. After drying the ethyl acetate phase with anhydrous sodium sulfate, it was concentrated, and 2-((5,6-dimethyl-1-(phenylcarbonyl)benzimidazole-2-ylthio)methyl)benzoic acid t-butyl ester was obtained (38 mg, yield quantitative).

2-((5,6-dimethyl-1-(phenylcarbonyl)benzimidazole-2-ylthio)methyl)benzoic acid t-butyl ester was dissolved in 1 ml of dichloromethane, to which trifluoroacetic acid (1 ml) was added and the mixture was stirred at room temperature for 6 hours. After the reaction was complete, the solvent was evaporated under reduced pressure and dried overnight to obtain the title compound (33 mg, yield quantitative).

The compound was confirmed by identification of molecular weight using LC-MS.

Calculated M=416.12, found (M+H)⁺=417.0

### Example 4

### Preparation of Compound No. 561

The title compound was obtained in a similar manner to Working Example 3.

The compound was confirmed by identification of molecular weight using LC-MS.

Calculated M=452.09, found (M+H)⁺=453.2

### Reference Example 4

### Preparation of 3-(naphthylmethyl)imidazolo(5,4-b)pyridine-2-thiol

To 2-amino-3-nitropyridine (1680 mg, 12 mmol) in a dimethylformamide (20 ml), sodium hydride (75 mg, 0.55 mmol) and 1-chloromethylnaphthalene (74 μl, 0.55 mmol) were added. After the resulting solution was stirred at 80° C. for 17 hours, water was added thereto, followed by extraction with ethyl ether. After drying the ethyl ether phase with anhydrous magnesium sulfate, it was concentrated, and the residue was purified by silica gel column chromatography (hexane:ethyl acetate=4:1) to obtain of naphthylmethyl(3-nitro(2-pyridil))amine (903 mg, yield 27%).

To naphthylmethyl(3-nitro(2-pyridil))amine (900 mg, 3.2 mmol) in ethanol (40 ml), 90.0 mg of 10% Pd—C was added. After the resulting solution was stirred in a hydrogen atmosphere at 50° C. for 8 hours, it was filtered through celite to remove Pd—C. The resulting solution was concentrated to obtain (3-amino(2-pyridil))naphthylmethylamine (860 mg, yield 99%). To the resulting (3-amino(2-pyridil))naphthylmethylamine (860 mg, 3.2 mmol) in ethanol (20 ml), carbon disulfide (6.1 ml, 102 mmol) was added. After the resulting solution was heated to reflux under stirring for 12 hours, it was allowed to stand at room temperature for 5 hours. The precipitate that deposited was filtered, and was washed three times with ethanol (5 ml). It was dried at 80° C. under reduced pressure for 5 hours to obtain the title compound (555 mg, yield 56%).

The compound was confirmed by identification of molecular weight using LC-MS.

Calculated M=291.08, found (M+H)⁺=292.3

### Reference Example 5

### Preparation of 3-((2,5-dimethylphenyl)methyl)imidazolo(5,4-b)pyridine-2-thiol

The title compound was synthesized in a similar manner to Reference Example 4.

The compound was confirmed by identification of molecular weight using LC-MS.

Calculated M=269.01, found (M+H)⁺=270.2

### Example 5

### Preparation of Compound No. 256

Using 3-(naphthylmethyl)imidazolo(5,4-b)pyridine-2-thiol (30 mg, 0.1 mmol) obtained in Reference Example 4 in a similar manner to Reference Example 2,2-((3-(naphthylmethyl)imidazolo(5,4-b)pyridine-2-ylthio)methyl)benzoic acid methyl ester was obtained (30 mg, yield 70%).

The 2-((3-(naphthylmethyl)imidazolo(5,4-b)pyridine-2-thio)methyl)benzoic acid methyl ester (30 mg, 0.068 mmol) thus obtained was subjected to hydrolysis in a similar manner to Example 1 to obtain the title compound (18.3 mg, yield 66%).

The compound was confirmed by identification of molecular weight using LC-MS.

Calculated M=425.12, found (M+H)⁺=426.1

### Example 6

The compounds in Table 74 were synthesized using the compounds obtained in Reference Examples 4 and 5 and various halide ester derivatives in a similar manner to Example 5.

The compounds were confirmed by identification of molecular weight using LC-MS.

TABLE 74

| Compound No. | Calculated M | Found (M + H)⁺ | Yield (Overall) % |
|---|---|---|---|
| 253 | 403.14 | 407.2 | 67 |
| 327 | 404.13 | 423.2 | 46 |
| 329 | 426.12 | 418.2 | 58 |
| 361 | 437.10 | 438.0 | 52 |
| 364 | 459.08 | 460.0 | 66 |

TABLE 75

| Compound No. | Calculated M | Found (M + H)⁺ | Yield (Overall) % |
|---|---|---|---|
| 321 | 428.13 | 429.2 | 27 |
| 354 | 461.10 | 462.2 | 20 |
| 460 | 379.14 | 380.2 | 19 |

TABLE 76

| Compound No. | Calculated M | Found (M + H)⁺ | Yield (Overall) % |
|---|---|---|---|
| 52 | 493.15 | 494.2 | 12 |
| 53 | 493.15 | 494.2 | 11 |

### Example 7

### Preparation of Compound No. 264

4-methyl-2-nitroaniline (913 mg, 6 mmol) was dissolved in acetonitrile (18 ml), to which anhydrous trifluoroacetic acid (1.00 ml, 7.2 mmol) was added and the mixture was subjected to reflux for 1.5 hours. After cooling to room temperature, it was concentrated under reduced pressure and dried to obtain 4-methyl-2-nitrotrifluoroacetanilide (1.396 g, yield 94%).

4-methyl-2-nitrotrifluoroacetanilide (1.396 g, 5.63 mmol) was dissolved in dimethylformamide (14 ml), and then potassium carbonate (940 mg, 6.80 mmol) and 1-chloromethylnaphthalene (1.15 g, 6.51 mmol) were sequentially added at room temperature and heated to 100° C. After 1 hour and 40 minutes, 5N aqueous sodium hydroxide solution (7.5 ml) was added and refluxed as it was for 15 minutes. After 15 minutes, it was cooled to room temperature, and water (180 ml) was added and stored at 4° C. overnight. The crystals that deposited were filtered and were dried to obtain ((1-naphthyl)methyl)(4-methyl-2-nitro-phenyl)amine (1.587 g, yield 96%).

To (1-naphthyl)methyl)(4-methyl-2-nitro-phenyl)amine (1.0021 g, 3.43 mmol), ethanol (5 ml) and 1,4-dioxane (5 ml) were added, and 2.058 M aqueous sodium hydroxide solution (1 ml) was further added, and refluxed in an oil bath. After 15 minutes, it was removed from the oil bath, and zinc powder (897 mg, 13.72 mmol) was fed thereto in portions. Then it was refluxed again in the oil bath for 2 hours. After 2 hours, it was concentrated under reduced pressure, and dissolved in ethyl acetate (50 ml), and washed twice with saturated saline (25 ml). After drying with magnesium sulfate, it was concentrated under reduced pressure and dried to obtain a brown oil of ((1-naphthyl)methyl)(2-amino-4-methyl-phenyl)amine (943.1 mg).

Subsequently, ((1-naphthyl)methyl)(2-amino-4-methyl-phenyl)amine (943.1 mg, 3.59 mmol) was dissolved in ethanol (6.4 ml), to which carbon bisulfide (7 ml, 116 mmol) was added, and then refluxed. After 10 hours, it was returned to room temperature, concentrated under reduced pressure. Ethanol (2 ml) was added to the residue, which was stirred at room temperature for 30 minutes, and was further stirred on ice for 30 minutes. The resulting crystals were filtered, and dried to obtain 1-((1-naphthyl)methyl)-6-methyl-benzimidazole-2-thiol (459.1 mg, yield 44%, 2 steps).

1-((1-naphthyl)methyl)-6-methyl-benzimidazole-2-thiol (431.1 mg, 1.42 mmol) was dissolved in dimethylformamide (12 ml), to which triethylamine (0.296 ml, 2.12 mmol) and 2-bromomethyl benzoic acid methyl ester (390.1 mg, 1.70 mmol) were added and heated to 80° C. After 5 hours and 50 minutes, triethylamine (0.296 ml, 2.12 mmol) and 2-bromomethyl benzoic acid methyl ester (325 mg, 1.42 mmol) were added, and heated for 1 hour and 10 minutes. Thereafter, it was concentrated under reduced pressure, and dissolved in ethyl acetate (80 ml), washed twice with water (30 ml), and dried in magnesium sulfate. The solvent was concentrated under reduced pressure. The residue was crystallized in ethyl acetate-hexane to obtain 410 mg, and the mother liquor was purified by silica gel column chromatography (hexane:ethyl acetate=6:1) to recover 87 mg of the same fraction as the crystals, with a total of 497 mg of 2-((1-((1-naphthyl)methyl)-6-methyl-benzimidazole-2-ylthio)methyl)benzoic acid methyl ester (yield 78%).

2-((1-((1-naphthyl)methyl)-6-methyl-benzimidazole-2-ylthio)methyl)benzoic acid methyl ester (497 mg, 1.098 mmol) was dissolved in methanol (10 ml) and tetrahydrofuran (10 ml), to which 4N aqueous lithium hydroxide solution (6.86 ml) was added. After stirring at room temperature for 2 hours and 30 minutes, saturated aqueous citric acid solution (10 ml) was added thereto to stop the reaction, and the mixture was concentrated under reduced pressure to reduce the amount of the solvent to about ⅓, which was dissolved in ethyl acetate (80 ml) and washed five times with water (20 ml). After concentrating the organic layer under reduced pressure, acetonitrile (10 ml) was added to the residue, which was again concentrated under reduced pressure, and the resulting crystals were filtered off and dried to obtain the title compound (439.1 mg, yield 91%).

The compound was confirmed by identification of molecular weight using LC-MS.

Calculated M=438.14, found (M+H)⁺=439.3

### Example 8

### Preparation of Compound No. 272

In a similar method to Working Example 7, the title compound was obtained.

The compound was confirmed by identification of molecular weight using LC-MS.

Calculated M=454.14, found (M+H)⁺=455.3

### Example 9

### Preparation of Compound No. 65

2-nitroaniline (829 mg, 6 mmol) and 1-methylindole carboxaldehyde (1242 mg, 7.8 mmol) were dissolved in 20 ml of tetrahydrofuran, to which acetic acid (200 μl) and NaBH(OAc)₃ (5087 mg, 24 mmol) were sequentially added and stirred at room temperature overnight. A saturated aqueous sodium hydrogen carbonate solution was added thereto, followed by extraction with ethyl acetate, dried with anhydrous sodium sulfate, and the solvent was evaporated. After purification by silica gel column chromatography (hexane:ethyl acetate=95:5), ((1-methylindole-3-yl)methyl)(2-nitrophenyl)amine was obtained (264 mg, yield 18%).

((1-methylindole-3-yl)methyl)(2-aminophenyl)amine (264 mg, 0.939 mmol) was dissolved in ethanol (10 ml), and Pd—C (50 mg, 10% Pd, 0.047 mmol) was added thereto, and stirred in hydrogen atmosphere at room temperature for 6 hours. After the reaction was complete, Pd—C was filtered off and the solvent was evaporated to obtain ((1-methylindole-3-yl)methyl)(2-aminophenyl)amine (212 mg, yield 90%).

((1-methylindole-3-yl)methyl)(2-aminophenyl)amine (212 mg, 0.845 mmol) was dissolved in pyridine (1 ml), and carbon bisulfide (1 ml, 16.9 mmol) was added thereto. The mixture was refluxed in nitrogen atmosphere for 1 hour. After the solvent was evaporated, it was purified by silica gel column chromatography (hexane:ethyl acetate=2:1) to obtain ((1-methylindole-3-yl)methyl)benzimidazole-2-thiol (96 mg, yield 39%).

Sodium hydride (12 mg, 0.342 mmol) and dimethylformamide (2 ml) were added to a previously dried reaction vessel. To the mixture were added ((1-methylindole-3-yl)methyl)benzimidazole-2-thiol (50 mg, 0.171 mmol) and 2-bromomethyl benzoic acid methyl ester (59 mg, 0.257 mmol), and then the mixture was stirred at 60° C. for 1 hour. Water was added thereto, followed by extraction with ethyl acetate. After the ethyl acetate phase was dried with anhydrous sodium sulfate, it was concentrated, and the residue was purified by silica gel column chromatography (hexane:ethyl acetate=2:1) to obtain 2-((1-((-methylindole-3-yl)methyl)benzimidazole-2-ylthio)methyl)benzoic acid methyl ester (54 mg, yield 74%).

To 2-((1-((1-methylindole-3-yl)methyl)benzimidazole-2-ylthio)methyl)benzoic acid methyl ester (54 mg, 0.122 mmol) in tetrahydrofuran (2 ml) and methanol (1 ml), 4N aqueous lithium hydroxide solution (0.5 ml) was added. After stirring at room temperature overnight, 6N hydrochloric acid was added to stop the reaction, followed by extraction with ethyl acetate. After washing the ethyl acetate phase with saturated saline, it was dried with anhydrous sodium sulfate. The solvent was evaporated under reduced pressure to obtain the title compound (48 mg, yield 92%).

The compound was confirmed by identification of molecular weight using LC-MS.

Calculated M=427.14, found (M+H)⁺=428.2

### Example 10

The compounds in the above Table 47 were synthesized using various halide ester derivatives in a similar manner to Working Example 9. The compounds were confirmed by identification of molecular weight using LC-MS.

### Example 11

### Preparation of Compound No. 51

Sodium hydride (104 mg, 2.86 mmol) and tetrahydrofuran (16 ml) were added to a previously dried reaction vessel. To the mixture were added 2-(benzimidazole-2-ylthiomethyl)benzoic acid methyl ester (428 mg, 1.43 mmol) and 2-(bromomethyl)benzoic acid t-butyl ester (466 mg, 3.46 mmol), and then the mixture was stirred at 60° C. for 50 minutes. Water was added thereto, followed by extraction with ethyl acetate. After the ethyl acetate phase was dried with anhydrous sodium sulfate, it was concentrated, and the residue was purified by silica gel column chromatography (hexane:ethyl acetate=3:1) to obtain 2-((1-((2-((t-butyl)oxycarbonyl)phenyl)methyl)benzimidazole-2-ylthio)methyl)benzoic acid methyl ester (495 mg, yield 71%).

To 2-((1-((2-((t-butyl)oxycarbonyl)phenyl)methyl)benzimidazole-2-ylthio)methyl)benzoic acid methyl ester (248 mg, 0.51 mmol), 4N hydrochloric acid in dioxane (1.28 ml, 5.1 mmol) was added, and stirred at room temperature overnight. After the solvent was evaporated, it was dried under reduced pressure to obtain 2-((2-((2-(methoxycarbonyl)phenyl)methylthio)benzimidazolyl)methyl)benzoic acid (220 mg, yield quantitative).

2-((2-((2-(methoxycarbonyl)phenyl)methylthio)benzimidazolyl)methyl)benzoic acid (180 mg, 0.42 mmol) was dissolved in chloroform (6 ml), to which HOBT (68 mg, 0.504 mmol), aniline (46 μl, 0.504 mmol), t-butanol (1.2 ml) and EDCI (97 mg, 0.504 mmol) were sequentially added and stirred overnight at room temperature. Water was added thereto, followed by extraction with dichloromethane. After drying with anhydrous sodium sulfate, it was filtered, and the solvent was evaporated. It was purified by silica gel column chromatography (hexane:ethyl acetate=3:2) to obtain 2-((1-((2-(N-phenylcarbamoyl)phenyl)methylthio)benzimidazole-2-ylthio)methyl)benzoic acid methyl ester (86 mg, yield 40%).

To the thus obtained 2-((1-((2-(N-phenylcarbamoyl)phenyl)methylthio)benzimidazole-2-ylthio)methyl)benzoic acid methyl ester (86 mg, 0.169 mmol) in tetrahydrofuran (2 ml) and methanol (1 ml), 4N aqueous lithium hydroxide solution (0.5 ml) was added, and stirred at 60° C. for about 2 hours. 6N aqueous hydrochloric acid solution was added to stop the reaction, which was extracted with ethyl acetate. After washing the ethyl acetate phase with saturated saline, it was dried with anhydrous sodium sulfate. The solvent was evaporated under reduced pressure to obtain the title compound (83 mg, yield quantitative).

The compound was confirmed by identification of molecular weight using LC-MS.

Calculated M=493.15, found (M+H)⁺=494.2

### Example 12

In a similar method to Working Example 11, the compounds shown in the above Table 48 were obtained using various benzoic acid ester derivatives.

The compounds were confirmed by identification of molecular weight using LC-MS.

### Example 13

### Preparation of Compound No. 619

Sodium hydride (400 mg, 10.0 mmol) and dimethylformamide (30 ml) were added to a previously dried reaction vessel. To the mixture were added 2-(benzimidazole-2-ylthiomethyl)benzoic acid methyl ester (1500 mg, 5.0 mmol) and bromoacetate t-butyl ester (1463 mg, 7.5 mmol), and the mixture was stirred at 80° C. for 2 hours. Water was added thereto, followed by extraction with ether. After the ether phase was dried with anhydrous sodium sulfate, it was concentrated, and the residue was purified by silica gel column chromatography (hexane:ethyl acetate=5:1) to obtain 2-(2-((2-(methoxycarbonyl)phenyl)methylthio)benzimidazolyl)acetic acid t-butyl ester (1298 mg, yield 63%).

To 2-(2-((2-(methoxycarbonyl)phenyl)methylthio)benzimidazolyl)acetic acid t-butyl ester (1290 mg, 3.13 mmol), trifluoroacetic acid (15 ml) was added, and stirred at room temperature overnight. After the solvent was evaporated, it was dried under reduced pressure to obtain 2-(2-((2-(methoxycarbonyl)phenyl)methylthio)benzimidazolyl)acetic acid (715 mg, yield 64%).

2-(2-((2-(methoxycarbonyl)phenyl)methylthio)benzimidazolyl)acetic acid (35 mg, 0.1 mmol) was dissolved in tetrahydrofuran (3 ml), to which aniline (11.2 mg, 0.12 mmol) and EDCI (23 mg, 0.12 mmol) were added, and then the mixture was stirred overnight at room temperature. Water was added thereto, followed by extraction with ethyl acetate. After drying with anhydrous sodium sulfate, it was filtered, the solvent was evaporated. The residue was purified by silica gel column chromatography (hexane:ethyl acetate=3:2) to obtain 2-((1-((N-phenylcarbamoyl)methyl)benzimidazole-2-ylthio)methyl)benzoic acid methyl ester (27.5 mg, yield 64%).

2-((1-((N-phenylcarbamoyl)methyl)benzimidazole-2-ylthio)methyl)benzoic acid methyl ester (20 mg, 0.046 mmol) thus obtained was subjected to hydrolysis as in Working Example 1 to obtain the title compound (6.9 mg, yield 36%).

The compound was confirmed by identification of molecular weight using LC-MS.

Calculated M=417.11, found (M+H)⁺=418.0

### Example 14

In a similar method to Example 13, the compounds shown in Table 77 were obtained using various aniline derivatives.

The compounds were confirmed by identification of molecular weight using LC-MS.

TABLE 77

| Compound No. | Calculated M | Found (M + H)⁺ | Yield (Overall) % |
|---|---|---|---|
| 622 | 431.13 | 432.3 | 5 |
| 621 | 431.13 | 432.3 | 5 |
| 620 | 431.13 | 432.3 | 21 |
| 637 | 447.13 | 448.2 | 13 |
| 636 | 117.13 | 448.1 | 23 |
| 635 | 447.13 | 448.3 | 44 |
| 642 | 442.11 | 443.2 | 27 |
| 657 | 467.13 | 488.1 | 19 |

TABLE 78

| Compound No. | Calculated M | Found (M + H)⁺ | Yield (Overall) % |
|---|---|---|---|
| 765 | 457.15 | 458.2 | 5 |
| 767 | 457.15 | 458.2 | 32 |

TABLE 79

| Compound No. | Calculated M | Found (M + H)⁺ | Yield (Overall) % |
|---|---|---|---|
| 866 | 434.13 | 435.2 | 76 |
| 869 | 456.11 | 457.3 | 83 |
| 904 | 468.09 | 469.1 | 52 |
| 937 | 436.15 | 437.2 | 61 |

TABLE 80

| Compound No. | Calculated M | Found (M + H)⁺ | Yield (Overall) % |
|---|---|---|---|
| 953 | 476.18 | 477.2 | 36 |
| 985 | 428.18 | 429.2 | 67 |
| 977 | 400.15 | 401.4 | 2 |

### Reference Example 6

### Preparation of 2-((1-(2-hydroxyethyl)-5,6-dimethylbenzimidazole-2-ylthio)]methyl)benzoic acid methyl ester

To 2-((5,6-dimethylbenzimidazole-2-ylthio)methyl) benzoic acid methyl ester (326 mg, 1 mmol) obtained in Reference Example 2 in dimethylformamide, potassium carbonate (207 mg, 1.5 mmol) and 2-bromoethanol (150 mg, 1.2 mmol) were added, and the resulting solution was stirred at 80° C. for 12 hours. After the reaction was complete, it was extracted with ether and the solvent was evaporated. The residue was purified by a flash column chromatography (hexane:ethyl acetate=4:1) to obtain the the title compound (248 mg, yield 67%).

The compound was confirmed by identification of molecular weight using LC-MS.

Calculated M=370.14, found (M+H)⁺=371.2

### Example 15

### Preparation of Compound No. 736

To 2-((1-(2-hydroxyethyl)-5,6-dimethylbenzimidazole-2-ylthio)methyl)benzoic acid methyl ester (45 mg, 0.23 mmol) in N-methylmorpholine (3 ml), Pph₃ (62 mg, 0.24 mmol) and DEAD (10.6 ml, 40% in toluene, 0.24 mmol) were added and the mixture was stirred at room temperature. After 10 minutes, phenol (11.3 mg, 0.12 mmol) was added thereto, which was stirred at room temperature for 12 hours. The solvent was evaporated and the residue was purified by thin layer chromatography (hexane:ethyl acetate=1:1) to obtain 2-((5,6-dimethyl-1-(2-phenoxyethyl)benzimidazole-2-ylthio)methyl)benzoic acid methyl ester (44 mg, yield 81%).

Using 2-((5,6-dimethyl-1-(2-phenoxyethyl)benzimidazole-2-ylthio)methyl)benzoic acid methyl ester (35 mg, 0.078 mmol) in a similar method to Example 1, a hydrolysis reaction was carried out to obtain the title compound (31 mg, yield 94%). The compound was confirmed by identification of molecular weight using LC-MS.

Calculated M=432.15, found (M+H)⁺=433.2

### Example 16

In a similar method to Example 15, the compounds shown in the above Table 78 were obtained using various phenol derivatives.

The compounds were confirmed by identification of molecular weight using LC-MS.

### Example 17

### Preparation of Compound No. 825

To an ester (33 mg, 0.075 mmol) of compound No. 68 obtained in Example 2 in dichloromethane, 50 to 60% m-chloroperbenzoic acid (26 mg, 0.083 mmol) was added while cooling on ice. After the resulting solution was stirred on ice for 2 hours, a saturated sodium hydrogen carbonate solution was poured and the solution obtained was extracted with chloroform. After washing the chloroform phase with water, it was concentrated and the residue was purified by thin layer chromatography (hexane:ethyl acetate=1:1) to obtain 2-(((5,6-dimethyl-1-(1-naphthylmethyl)benzimidazole-2-yl)sulfinyl)methyl)benzoic acid methyl ester (7.1 mg, yield 21%).

In a manner similar to Example 1, this was subjected to hydrolysis to obtain the title compound (5.2 mg, yield 76%).

The compound was confirmed by identification of molecular weight using LC-MS.

Calculated M=440.12, found (M+H)⁺=441.3

### Example 18

### Preparation of Compound No. 869

To an ester (39 mg, 0.094 mmol) of compound No. 37 obtained in Example 2 in dichloromethane (5 ml), 50 to 60% m-chloroperbenzoic acid (64 mg, 0.374 mmol) was added while cooling on ice. After the resulting solution was stirred at room temperature for 4 hours, a saturated sodium hydrogen carbonate solution was poured and the solution obtained was extracted with chloroform. After washing the chloroform phase with water, it was concentrated and the residue was purified by flash layer chromatography (hexane:ethyl acetate=5:1) to obtain 2-(((1-((2,5-dimethylphenyl)methyl)benzimidazole-2-yl)sulfonyl)methyl)benzoic acid methyl ester (37 mg, yield 87%).

In a manner similar to Example 1,2-(((1-((2,5-dimethylphenyl)methyl)benzimidazole-2-yl)sulfonyl)methyl)benzoic acid methyl ester (64 mg, 0.14 mmol) was subjected to hydrolysis to obtain the title compound (53 mg, yield 87%).

The compound was confirmed by identification of molecular weight using LC-MS.

Calculated M=434.13, measured (M+H)⁺=435.2

### Example 19

In a manner similar to Example 18, the compounds shown in the above Table 51 were synthesized using the esters of the compounds obtained in Working Example 2. The compounds were confirmed by identification of molecular weight using LC-MS.

### Example 20

### Preparation of Compound No. 952

To 5,6-dimethylbenzimidazole-2-thiol (713 mg, 4 mmol) in dimethylformamide (10 ml), triethylamine (836 μl, 6 mmol) and 2-bromomethylbenzonitrile (1176 mg, 6 mmol) were added. After stirring at 80° C. overnight, water was added to the mixture, followed by extraction with ethyl acetate. After the ethyl acetate phase was dried with anhydrous sodium sulfate, it was concentrated and the residue was purified by silica gel column chromatography (hexane:ethyl acetate=3:2) to obtain 2-((5,6-dimethylbenzimidazole-2-ylthio)methyl)benzenecarbonitrile (1159 mg, yield 99%).

Sodium hydride (178 mg, 4.90 mmol) and tetrahydrofuran (30 ml) were added to a previously dried reaction vessel. To the mixture were added 2-((5,6-dimethylbenzimidazole-2-ylthio)methyl)benzenecarbonitrile (719 mg, 2.45 mmol) and 2,5-dichlorobenzyl chloride (543 μl, 4.90 mmol), and the mixture was stirred at 60° C. for 40 minutes. Water was added thereto, followed by extraction with ethyl acetate. After the ethyl acetate phase was dried with anhydrous sodium sulfate, it was concentrated, and the residue was purified by silica gel column chromatography (hexane:ethyl acetate=3:1) to obtain 2-((1-((2,5-dimethylphenyl)methyl)-5,6-dimethylbenzimidazole-2-ylthio)methyl)benzenecarbonitrile (370 mg, yield 37%).

2-((1-((2,5-dimethylphenyl)methyl)-5,6-dimethylbenzimidazole-2-ylthio)methyl)benzenecarbonitrile (165 mg, 0.401 mmol) was dissolved in toluene (3 ml), to which Me₃SnN₃ (124 mg, 0.602 mmol) was added, and refluxed in nitrogen atmosphere overnight. After the reaction was complete, the solvent was evaporated, and the residue was purifed by silica gel column chromatography (dichloromethane:methanol=19:1) to obtain the title compound (75 mg, yield 41%).

The compound was confirmed by identification of molecular weight using LC-MS.

Calculated M=454.19, found (M+H)⁺=455.2

### Example 21

In a manner similar to Example 20, the compounds shown in the above Table 80 were obtained.

The compounds were confirmed by identification of molecular weight using LC-MS.

### Reference Example 7

### Production of 4-(5,6-dimethylbenzimidazole-2-ylthio)butanoate ethyl ester

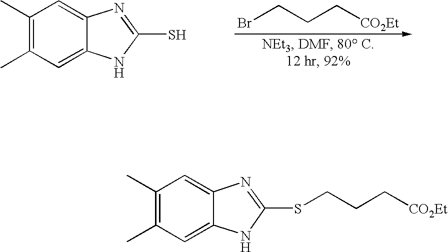

35 μl (0.25 mmol) of triethylamine and 36 μl (0.25 mmol) of 4-bromobutanoate ethyl ester were added to 36 mg (0.20 mmol) of the obtained 5,6-dimethylbenzimidazole-2-thiol. After stirring the resulting solution for 12 hours at 80° C., water was added followed by extraction with diethyl ether. After drying the diethyl ether phase with anhydrous magnesium sulfate, it was concentrated and residue was purified by silica gel column chromatography (hexane:ethyl acetate=4:1) to obtain 54 mg of the target compound (yield: 92%). Confirmation of the compound was carried out by identifying it from the molecular weight using LC-MS.

Calculated value M=292.12, Measured value (M+H)⁺=293.40

### Reference Example 8

The following compounds were synthesized according to the same method as Reference Example 7.

Confirmation of the compounds was carried out by identifying them from the molecular weight using LC-MS.

4-(benzimidazole-2-ylthio)butanoate ethyl ester

Calculated value M=264.09, Measured value (M+H)⁺=293.40

4-(5,6-difluorobenzimidazole-2-ylthio)butanoate ethyl ester

Calculated value M=300.07, Measured value (M+H)⁺=301.3

### Reference Example 9

### Production of 3-bromomethyl-5-methylbenzo[b]thiophene

Step 1

### Production of 3-hydroxymethyl-β-nitrotoluene

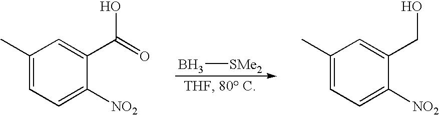

5.02 g (27.7 mmol) of 5-methyl-2-nitrobenzoic acid were dissolved in 20 ml of THF followed by dropping in 11.1 ml of 10.2 M borane dimethylsulfide complex and heating at 80° C. After 1.5 hours, 30 ml of 1 M hydrochloric acid were dropped into this reaction system while cooling with ice and stirring. The system was then concentrated under reduced pressure to obtain 100 ml of the aqueous phase followed by extraction with ethyl acetate (100 ml×2). After washing the ethyl acetate phase with saturated brine, the organic phase was dried with magnesium sulfate followed by concentration under reduced pressure and drying to obtain 3.91 g of the target compound (yield: 85%).

Step 2

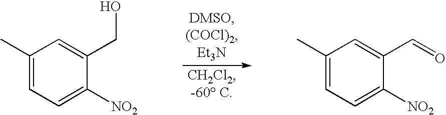

### Production of 3-formyl-β-nitrotoluene

5.5 ml (63.2 mmol) of oxalyl chloride were added to 50 ml of dichloromethane and cooled to −60° C. After 20 minutes, 9.13 ml (138.6 mmol) of DMSO were added and stirred at −60° C. followed 15 minutes later by the addition of 3.91 g (23.3 mmol) of the 3-hydroxymethyl-p-nitrotoluene obtained in Step 1 at −60° C. and stirring. After 30 minutes, 45 ml of triethylamine were dropped in at −60° C. and then returned to room temperature. After concentrating under reduced pressure, 0.1 M hydrochloric acid was added to the residue followed by extraction with ethyl acetate (150 ml×2). The organic phase was then dried with magnesium sulfate and concentrated under reduced pressure to obtain 5.02 g of the target compound (crude yield: 130%).

Step 3

### Production of 2-carboxyethyl-5-methylbenzo[b]thiophene

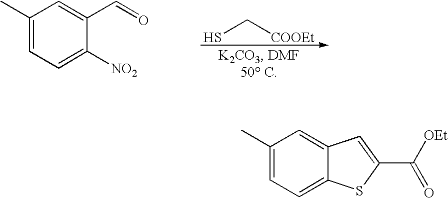

5.02 g (63.2 mmol) of the 3-formyl-p-nitrotoluene obtained in Step 2 were dissolved in 50 ml of DMF followed by the addition of 3.06 ml (28.1 mmol) of ethyl mercaptoacetate and 4.85 g (35.1 mmol) of potassium carbonate and stirring at 50° C. After 9.5 hours, the temperature was raised to 80° C. followed by additional heating for 100 minutes. Following completion of the reaction, 250 ml of water were added to the reaction solution followed by extraction with ethyl acetate (100 ml×3) and drying with magnesium sulfate. After concentrating the solvent under reduced pressure, the residue was purified by silica gel column chromatography (hexane:ethyl acetate=8:1) followed by additionally purifying by silica gel column chromatography (hexane:ethyl acetate=10:1) to obtain 2.48 g (11.2 mmol) of the target compound (yield: 48%).

¹H-NMR (400 MHz, CDCl₃) (ppm): 7.98 (s, 1H), 7.73 (d, 1H, J=8.28 Hz), 7.65 (s, 1H), 7.27 (d, 1H, J=8.32 Hz), 4.39 (q, 2H), 2.47 (s, 3H), 1.41 (s, 3H)

Step 4

### Production of 2-carboxy-5-methylbenzo[b]thiophene

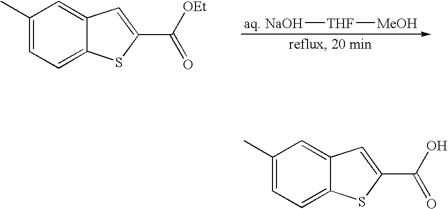

30 ml of a solution of methanol, THF and 2 M aqueous sodium hydroxide solution (1:1:1) were added to 2.17 g (9.87 mmol) of the 2-carboxyethyl-5-methylbenzo[b]thiophene obtained in Step 3 and refluxed. After 20 minutes, the solution was neutralized with acid followed by concentration under reduced pressure and recovery of the precipitate. This was then washed with 50 ml of water and dried to obtain 2.03 g (10.5 mmol) of the target compound (crude yield: 107%).

¹H-NMR (400 MHz, CDCl₃) (ppm): 7.94 (s, 1H), 7.74 (d, 1H, J=8.56 Hz), 7.69 (s, 1H), 7.27 (d, 1H, J=8.30 Hz), 2.47 (s, 3H)

Step 5

### Production of 5-methylbenzo[b]thiophene

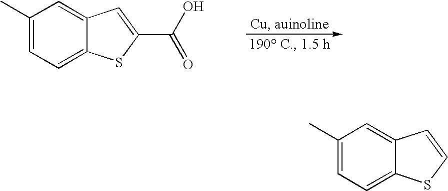

2.03 g (9.87 mmol) of the 2-carboxy-5-methylbenzo[b]thiophene obtained in Step 4 were dissolved in 10 ml of quinoline followed by the addition of 799.2 mg of copper powder and heating to 190° C. After 100 minutes, the solution was cooled followed by the addition of 40 ml of 0.5 M hydrochloric acid and extraction with ethyl acetate (40 ml×2). The organic phase was washed with 40 ml of water and then dried with magnesium sulfate. After concentrating the solvent under reduced pressure, it was purified by silica gel column chromatography (hexane:ethyl acetate=20:1) to obtain 1.41 g (9.51 mmol) of the target compound (yield of the two steps from Step 4: 96%).

¹H-NMR (270 MHz, CDCl₃) (ppm): 7.76 (d, 1H, J=8.24 Hz), 7.62 (s, 1H), 7.40 (d, 1H, J=5.44 Hz), 7.24 (m, 1H), 7.17 (d, 1H, J=8.24 Hz), 2.47 (s, 3H)

Step 6

### Production of 3-chloromethylcarbonyl-5-methylbenzo[b]thiophene

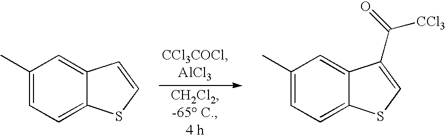

10 ml of dichloromethane were added to 1.33 g (9.97 mmol) of aluminum trichloride followed by cooling to −65° C. with dry ice and acetone. After 10 minutes, 1.12 ml (10.0 mmol) of trichloroacetylchloride were dropped in. After an additional 20 minutes, 10 ml of dichloromethane solution containing 1.41 g (9.51 mmol) of the 5-methylbenzo[b]thiophene obtained in Step 5 were dropped in and then stirred at about −65° C. After 1 hour and 40 minutes, the temperature was raised to −40° C. After an additional 1 hour and 10 minutes, the temperature was raised to 0° C. After another 1 hour and 40 minutes, 10 ml of 1 M hydrochloric acid were added and stirred. After adding 20 ml of water to the reaction system, removing the dichloromethane phase by a liquid separation procedure and then additionally extracting the aqueous phase with ethyl acetate, the aqueous phase was combined with the dichloromethane phase and then concentrated under reduced pressure. 3.2 g of the resulting residue were purified by silica gel column chromatography (silica gel: 120 g, hexane) to obtain 686.7 mg (2.34 mmol) of the target compound (yield: 24%).

¹H-NMR (400 MHz, CDCl₃) (ppm): 8.89 (s, 1H), 8.51 (s, 1H), 7.78 (d, 1H, J=8.28 Hz), 7.30 (d, 1H, J=8.32 Hz), 2.53 (s, 3H)

Step 7

### Production of 3-carboxy-5-methylbenzo[b]thiophene

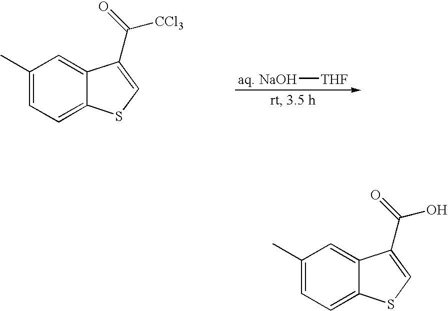

686.7 mg (2.34 mmol) of the 3-chloromethylcarbonyl-5-methylbenzo[b]thiophene obtained in Step 6 were dissolved in 2.0 ml of THF and 3.0 ml of MeOH followed by the addition of 2 ml of 2 M aqueous sodium hydroxide solution and stirring at room temperature. After 2 hours and 45 minutes, 5 ml of 2 M aqueous sodium hydroxide solution were added followed by heating to 60° C. After cooling 30 minutes later and adding 10 ml of 2 M hydrochloric acid and 30 ml of water, the solution was extracted with ethyl acetate followed by concentration under reduced pressure and drying to obtain 438.9 mg (2.28 mmol) of the target compound (yield: 97%).

¹H-NMR (400 MHz, CDCl₃) (ppm): 8.44 (s, 1H), 8.36 (s, 1H), 7.74 (d, 1H, J=8.04 Hz), 7.22 (d, 1H, J=8.28 Hz), 2.50 (s, 3H)

Step 8

### Production of 3-hydroxymethyl-5-methylbenzo[b]thiophene

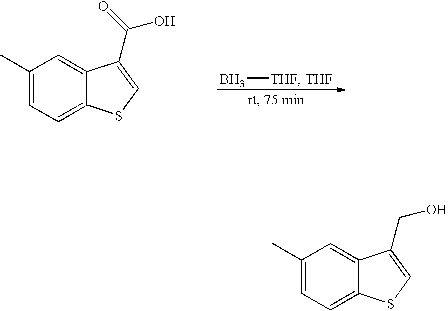

438.9 mg (2.28 mmol) of the 3-carboxy-5-methylbenzo[b]thiophene obtained in Step 7 were dissolved in 5 ml of THF followed by the addition of BH₃.THF complex solution and stirring at room temperature. After 1 hour and 15 minutes, 4 ml of 2 M hydrochloric acid were added and stirred followed by the addition of 50 ml of ethyl acetate. The organic phase was washed with 30 ml of water and dried with magnesium sulfate followed by concentration under reduced pressure. The resulting residue was purified with Biotage (hexane:ethyl acetate=4:1) to obtain 347.6 mg (1.95 mmol) of the target compound (yield: 86%)

¹H-NMR (400 MHz, CDCl₃) (ppm): 7.74 (d, 1H, J=8.04 Hz), 7.65 (s, 1H), 7.34 (s, 1H), 7.19 (d, 1H, J=8.28 Hz), 4.89 (s, 2H), 2.48 (s, 3H)

Step 9

### Production of 3-bromomethyl-5-methylbenzo[b]thiophene

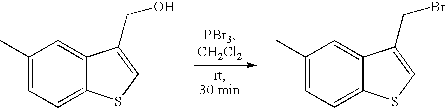

326 mg (1.83 mmol) of the 3-hydroxymethyl-5-methylbenzo[b]thiophene obtained in Step 8 were dissolved in 10 ml of dichloromethane followed by the addition of 0.262 ml of phosphorous tribromide and stirring at room temperature. After 30 minutes, 30 ml of water were added followed by stirring for 10 minutes and extracting with dichloromethane (30 ml×2). The organic phase was then concentrated under reduced pressure and dried to obtain 397.5 mg (1.65 mmol) of the target compound (yield: 90%).

¹H-NMR (270 MHz, CDCl₃) (ppm): 7.74-7.67 (m, 2H), 7.46 (s, 1H), 7.22 (d, 1H, J=8.24 Hz), 4.74 (s, 2H), 2.51 (s, 3H)

### Reference Example 10

### Production of ((4-methylbenzo[b]thiophene-3-yl)methyl)trimethylammonium iodide

Step 1

### Production of 2-cyano-3-nitrotoluene

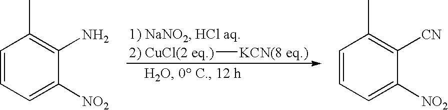

76.07 g (500 mmol) of 2-amino-3-nitrotoluene were added to 100 g (990 mmol) of 36% hydrochloric acid and 500 g of ice followed by stirring vigorously at 0° C. 80 ml of an aqueous solution containing 37.95 g (550 mmol) of sodium nitrite was then slowly dropped in while holding the temperature to 0-5° C. Following completion of dropping, 100 ml of toluene were added followed by stirring for 30 minutes at 0° C. The reaction solution was placed in an ice-NaCl bath followed by slowly adding sodium bicarbonate while stirring vigorously to neutralize the pH to about 6 (diazonium salt solution (1)).

An aqueous solution (550 ml) containing 260.5 g (4000 mmol) of potassium cyanide was slowly added at 0° C. to an aqueous solution (650 ml) containing 99.0 g (1000 mmol) of copper (I) chloride followed by stirring for 90 minutes and then adding 200 ml of ethyl acetate. The diazonium salt solution (1) prepared above was then dropped into this solution over the course of 30 minutes while holding the temperature to 0-5° C. The solution was then stirred for 12 hours in an ice bath and then warmed to room temperature. After extracting the reaction solution with ethyl acetate and washing the organic phase with water, it was dried with magnesium sulfate followed by concentrating the solvent under reduced pressure. The residue was then purified by silica gel column chromatography (hexane:ethyl acetate=20:1→10:1→7:1→5:1→3:1) to obtain 58.63 g (362 mmol) of the target compound (yield: 72%).

¹H-NMR (270 MHz, CDCl₃) (ppm): 7.68 (2H, m), 8.13 (1H, m), 2.715 (3H, s)

Step 2

### Production of 3-amino-2-ethoxycarbonyl-4-methylbenzo[b]thiophene

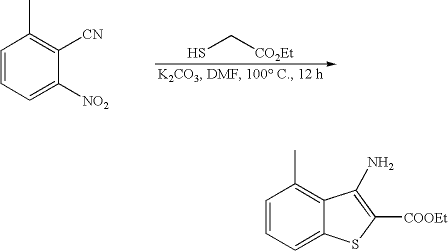

A DMF solution (250 ml) containing 58.63 g (362 mmol) of the 2-cyano-3-nitrotoluene obtained in Step 1, 47.5 g (395 mmol) of ethyl 2-mercaptoacetate and 57.5 g (416 mmol) of potassium carbonate was stirred for 12 hours at 100° C. The reaction solution was then concentrated, as is, under reduced pressure to remove the DMF to a certain degree. Water was added to dissolve inorganic substances followed by extraction with ethyl acetate. After washing the organic phase with water, it was dried with magnesium sulfate followed by concentration of the solvent under reduced pressure. The residue was then purified by silica gel column chromatography (hexane:ethyl acetate=10:1) to obtain 62.86 g (267 mmol) of the target compound (yield: 74%).

¹H-NMR (270 MHz, CDCl₃) (ppm): 7.54 (d, 1H,), 7.29 (t, 1H), 7.03 (d, 1H), 6.28 (s, 2H), 4.35 (q, 2H), 2.82 (s, 3H), 1.39 (t, 3H)

Step 3

### Production of 3-cyano-2-ethoxycarbonyl-4-methylbenzo[b]thiophene

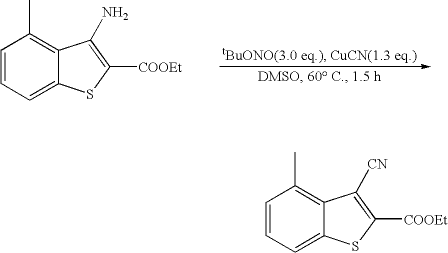

After replacing the reaction system with nitrogen, 82.0 g (795 mmol) of t-butyl nitrite and 30.9 g (345 mmol) of copper cyanide were added to 250 ml of DMSO and dissolved by stirring for 30 minutes at 55° C. Moreover, a DMSO solution (100 ml) containing 62.2 g (265 mmol) of the 3-amino-2-ethoxycarbonyl-4-methylbenzo[b]thiophene obtained in Step 2 was slowly dropped in over the course of 2 hours while holding the temperature at 55° C. After warming the reaction solution to 60° C. and stirring for 140 minutes, it was cooled to 0° C. followed by slowly adding water and stirring for 1 hour at 0° C. The reaction solution was then filtered with Celite to remove impurities, and after extracting with dichloromethane and washing the organic phase with water, it was dried with magnesium sulfate followed by concentrating the solvent under reduced pressure. The residue was then purified by silica gel column chromatography (hexane:ethyl acetate=20:1→15:1→10:1) to obtain 15.59 g (63.6 mmol) of the target compound (yield: 24%).

¹H-NMR (270 MHz, CDCl₃) (ppm): 7.73 (d, 1H), 7.44 (t, 1H), 7.30 (d, 1H), 4.50 (q, 2H), 2.95 (s, 3H), 1.47 (t, 3H)

Step 4

### Production of 3-cyano-4-methylbenzo[b]thiophene

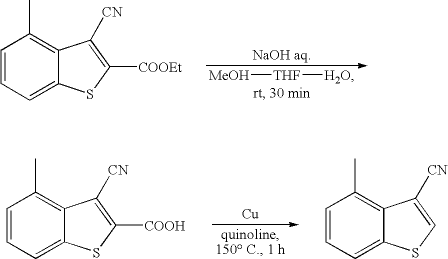

15.59 g (63.6 mmol) of the 3-cyano-2-ethoxycarbonyl-4-methylbenzo[b]thiophene obtained in Step 3 were dissolved in a mixture of methanol (150 ml), THF (150 ml) and water (150 ml) followed by the addition of 30 ml of 5 M aqueous sodium hydroxide solution and stirring for 2 hours at room temperature. After concentrating the solvent under reduced pressure, the pH was lowered to 4 by addition of 1 M hydrochloric acid and, after extracting with ethyl acetate and washing the organic phase with water, it was dried with magnesium sulfate. The solvent was then concentrated under reduced pressure to obtain 3-cyano-2-carboxy-4-methylbenzo[b]thiophene. This and 1.27 g (20 mmol) of copper powder were added to 18 ml of quinoline followed by stirring for 2 hours at 150° C. After cooling the reaction solution, it was filtered with Celite and the pH of the filtrate was lowered to 3 by addition of hydrochloric acid to transfer the quinoline as the solvent to the aqueous phase followed by extraction with ethyl acetate. After washing the organic phase with water, it was dried with magnesium sulfate and the solvent was concentrated under reduced pressure. The residue was then purified by silica gel column chromatography (hexane:ethyl acetate=20:1) to obtain 9.10 g (52.6 mmol) of the target compound (yield of the two steps: 83%).

¹H-NMR (270 MHz, CDCl₃) (ppm): 8.15 (s, 1H), 7.74 (d, 1H), 7.36 (t, 1H), 7.25 (d, 1H), 2.91 (s, 3H)

Step 5

### Production of 3-((N,N-dimethylamino)methyl)-4-methylbenzo[b]thiophene

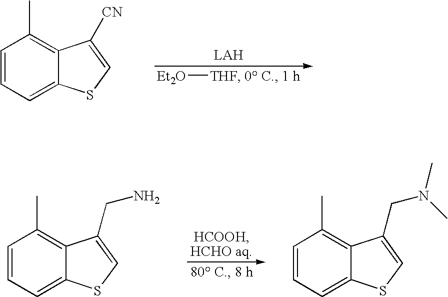

After dropping a diethyl ether (20 ml) and THF (20 ml) solution containing 9.10 g (52.6 mmol) of the 3-cyano-4-methylbenzo[b]thiophene obtained in Step 4 into 50 ml of a diethyl ether suspension of 2.0 g (53 mmol) of lithium aluminum hydride over the course of 15 minutes at 0° C., the solution was stirred for 30 minutes at room temperature. Following completion of the reaction, excess LAH in the reaction solution was treated with hydrochloric acid followed by the addition of aqueous sodium hydroxide solution to make alkaline. After saturating the aqueous phase with potassium carbonate, extracting with dichloromethane and washing the organic phase with water, it was dried with magnesium sulfate. The solvent was then concentrated under reduced pressure to obtain 3-aminomethyl-4-methylbenzo[b]thiophene. 11.5 (250 mmol) of formic acid and 10.0 g (123 mmol) of 37% aqueous formaldehyde solution were sequentially added to this followed by stirring for 5 hours at 80° C. Following completion of the reaction, after adding aqueous hydrochloric acid solution to the reaction solution, it was concentrated under reduced pressure to remove the formic acid and formaldehyde. Aqueous sodium hydroxide solution was then added to make the solution alkaline followed by extraction with dichloromethane. After washing the organic phase with water, it was dried with magnesium sulfate and the solvent was concentrated under reduced pressure. The residue was then purified by silica gel column chromatography (hexane:ethyl acetate=10:1) to obtain 2.61 g (12.8 mmol) of the target compound (yield of the two steps: 24%). Confirmation of the compound was carried out by identifying from ¹H-NMR.

¹H-NMR (270 MHz, CDCl₃) (ppm): 7.66 (s, 1H), 7.26-7.09 (m, 3H), 3.65 (s, 2H), 2.85 (s, 3H), 2.27 (s, 6H)

Step 6

### Production of ((4-methylbenzo[b]thiophene-3-yl)methyl)trimethylammonium iodide

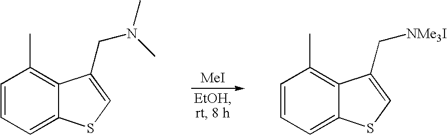

3.69 g (26 mmol) of methyl iodide were added to 20 ml of an ethanol solution containing 2.61 g (12.8 mmol) of the 3-((N,N-dimethylamino)methyl)-4-methylbenzo[b]thiophene obtained in Step 5 followed by stirring for 18 hours at room temperature. As this results in a white suspension, after filtering out the excess methyl iodide and solvent, it was washed with ethanol (10 ml×2) and diethyl ether (10 ml×3) to obtain 3.08 g (8.88 mmol) of the target compound in the form of a white solid (yield: 69%).

¹H-NMR (270 MHz, DMSO)(ppm): 8.19 (s, 1H), 7.93 (d, 1H), 7.36-7.25 (m, 2H), 4.91 (s, 2H), 3.05 (s, 9H), 2.77 (s, 3H)

### Reference Example 11

### Production ((1,4-dimethylindole-3 yl)methyl)methylammonium iodide

Step 1

### Production of 4-methylindole

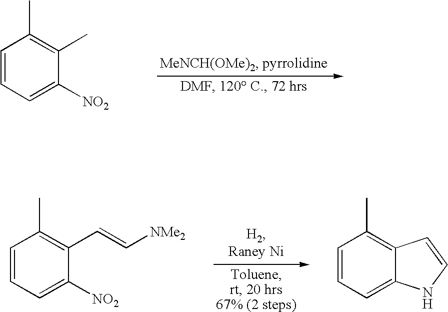

30.5 g (256 mmol) of N,N-dimethylformamidedimethylacetal and 10.9 g (153 mmol) of pyrrolidine were added to 150 ml of an N,N-dimethylformamide solution containing 19.4 g (128 mmol) of 2,3-dimethylnitrobenzene. After stirring the resulting solution for 72 hours at 120° C., it was concentrated as is. 100 ml of toluene were added to the resulting brown oily substance followed by the addition of 11 g of Raney nickel (50%, aqueous slurry, pH >9) and stirring. The inside of the reaction vessel was replaced with hydrogen gas followed by stirring for 20 hours at room temperature in a hydrogen gas atmosphere. After filtering the reaction solution with Celite, the filtrate was concentrated to obtain 30 g of a black solution. This was then purified by silica gel column chromatography (hexane:ethyl acetate=10:1) to obtain 11.33 g (86 mmol) of the target compound (yield of the two steps: 67%). Confirmation of the compound was carried out by identifying using ¹H-NMR.

¹H-NMR (270 MHz, CDCl₃) (ppm): 7.28-7.07 (m, 3H), 6.93 (m, 1H), 6.57 (m, 1H), 2.57 (s, 3H)

Step 2

Production of 1,4-dimethylindole

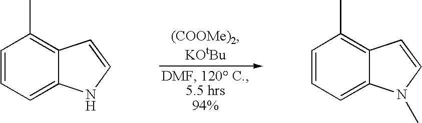

12.7 g (134 mmol) oft-butoxypotassium and 80 ml of N,N-dimethylformamide were added to a pre-dried reaction vessel. 8.9 g (67.9 mmol) of the 4-methylindole obtained in Step 1 were added followed by stirring for 35 minutes at room temperature. 15.8 g (134 mmol) of dimethyl oxalate were added to this followed by stirring for 5 hours and 30 minutes at 120° C. After concentrating under reduced pressure, 200 ml of water were added followed by treatment with 1 M hydrochloric acid to make acidic (pH=3) followed by extraction with ethyl acetate (200 ml×2) and drying with anhydrous magnesium sulfate. After distilling off the solvent under reduced pressure, it was purified by silica gel column chromatography (hexane:ethyl acetate=5:1) to obtain 9.24 g (53 mmol) of the target compound (yield: 94%). Confirmation of the compound was carried out by identifying using ¹H-NMR.

¹H-NMR (270 MHz, CDCl₃) (ppm): 7.25-7.09 (m, 2H), 7.03 (m, 1H), 6.90 (m, 1H), 6.49 (m, 1H), 3.78 (s, 3H), 2.55 (s, 3H)

Step 3

### Production of 1,4-dimethyl-3-(N,N-dimethylaminomethyl)indole

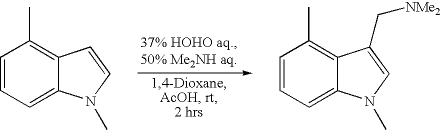

5.9 ml (72.0 mmol) of 37% aqueous formaldehyde solution and 7.08 ml (78 mmol) of 50% aqueous dimethylamine solution were sequentially added to a mixed system containing 25 ml each of 1,4-dioxane and acetic acid. After cooling to room temperature, as this reaction generates heat, 10 ml of a 1,4-dioxane solution containing 9.24 g (63.6 mmol) of the 1,4-dimethylindole obtained in Step 2 were added followed by stirring for 2 hours at room temperature. The reaction solution was then concentrated as is. 5 M aqueous sodium hydroxide solution were then added to the residue to make alkaline (pH=12) and bring to a total volume of 100 ml followed by extraction with ethyl acetate (100 ml×2). The organic phase was then dried with anhydrous magnesium sulfate and concentrated under reduced pressure to obtain 12.93 g (63.9 mmol) of the target compound (crude yield: 100.4%). Confirmation of the compound was carried out by identifying using ¹H-NMR.

¹H-NMR (270 MHz, CDCl₃) (ppm): 7.15-7.06 (m, 2H), 6.91 (m, 1H), 6.85 (m, 1H), 3.71 (s, 3H), 3.59 (s, 2H), 2.74 (s, 3H), 2.27 (s, 6H)

Step 4

### Production of ((1,4-dimethylindole-3-yl)methyl)trimethylammonium iodide

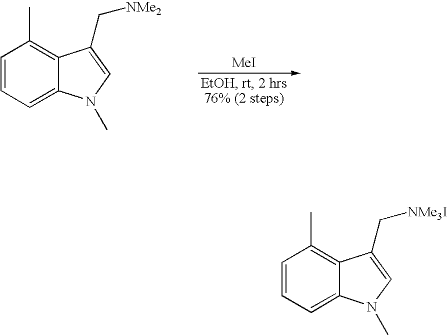

12.93 g (63.6 mmol) of the 1,4-dimethyl-3-(N,N-dimethylaminomethyl)indole obtained in Step 3 were dissolved in 60 ml of ethanol followed by the addition of 4.36 ml (70 mmol) of methyl iodide. A white precipitate formed after stirring for 2 hours at room temperature. This was then filtered, washed twice with 10 ml of ethanol and dried in a vacuum to obtain 16.66 g (48.4 mmol) of the target compound (yield of the two steps: 76%). Confirmation of the compound was carried out by identifying using ¹H-NMR.

¹H-NMR (270 MHz, DMSO) (ppm): 7.65 (s, 1H), 7.36 (d, 1H), 7.13 (t, 1H), 6.91 (d, 1H), 4.74 (s, 2H), 3.82 (s, 3H), 3.01 (s, 9H), 2.65 (s, 3H)

### Reference Example 12

### Production of 4-(5-methoxybenzimidazole-2-ylthio)butanoate ester hydrogen bromide salt

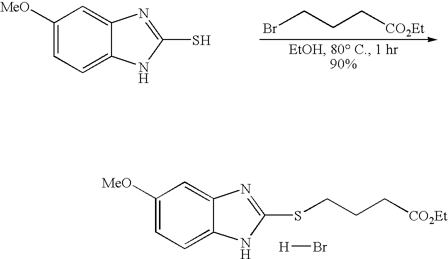

6.48 g (33.2 mmol) of 4-bromobutanoate ethyl ester were added to 10 ml of an ethanol solution containing 5.0 g (27.7 mmol) of 5-methoxybenzimidazole-2-thiol followed by stirring for 1 hour at 80° C. and adding 90 ml of ethyl acetate. The reaction solution was returned to room temperature and the formed crystals were filtered out followed by drying to obtain 9.34 g of the target compound (yield: 90%).

¹H-NMR (270 MHz, CDCl₃) (ppm): 7.65 (d, 1H, J=8.91 Hz), 7.24 (s, 1H), 7.00 (dd, 1H, J=2.43, 8.91 Hz), 4.21 (q, 2H, J=7.29 Hz), 3.83 (s, 3H), 3.74 (m, 2H), 2.61 (m, 2H), 2.10 (m, 2H), 1.30 (t, 3H, J=7.29 Hz)

### Example 22

### Production of Compound No. 1027

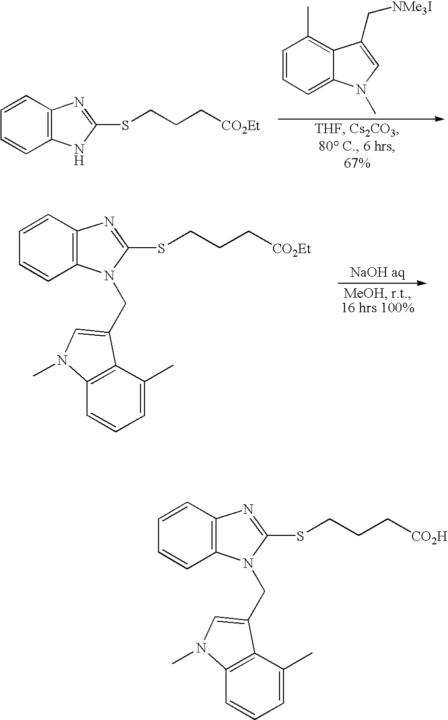

480 mg (2.49 mmol) and 10 ml of tetrahydrofuran were added to a pre-dried reaction vessel. 505 mg (1.91 mmol) of the 4-(benzimidazole-2ylthio)butanoate ethyl ester obtained in Reference Example 8 and 724 mg (2.10 mmol) of ((1,4-dimethylindole-3-yl)methyl)trimethylammonium iodide were added followed by stirring for 6 hours at 80° C. After filtering the solution by passing through Celite, it was concentrated under reduced pressure. The residue was then purified by silica gel column chromatography (dichloromethane:ethyl acetate=8:1) to obtain 540 mg (1.28 mmol) of 4-(1-((1,4-dimethylindole-3-yl)methyl)benzimidazole-2-ylthio)butanoate ethyl ester (yield: 67%).

2.0 ml of a 2M aqueous sodium hydroxide solution were then added to 6 ml of a methanol solution containing 540 mg (1.28 mmol) of the resulting 4-(1-((1,4-dimethylindole-3-yl)methyl) benzimidazole-2-ylthio)butanoate ethyl ester. After stirring for 16 hours at room temperature, 6 M hydrochloric acid was added to stop the reaction. The solvent was removed to a certain degree by concentration under reduced pressure followed by extraction with ethyl acetate. After washing the ethyl acetate phase with saturated brine, it was dried with anhydrous magnesium sulfate. After distilling off the solvent under reduced pressure, it was purified by silica gel column chromatography (dichloromethane:methanol=8:1) to obtain 502 mg (1.28 mmol) of the target compound (yield: 100%). Confirmation of the compound was carried out by identifying from its molecular weight using LC-MS.

Calculated value M=393.15, Measured value (M+H)⁺=394.2

### Example 23

The following compounds and the compounds in the following table were synthesized according to the same method as Example 25 using the compounds indicated in Reference Example 7 or 8 as well as various quaternary ammonium salts or halide derivatives synthesized with reference to Reference Examples 9-11 and other references described in the text. Confirmation of the compounds was carried out by identifying from their molecular weights using LC-MS. However, some of the compounds were synthesized using conditions that somewhat differed from those of Example 25, including conditions such as the use of DMF and so forth for the solvent and the use of potassium carbonate for the base in coupling, the use of THF and EtOH for the solvent in hydrolysis, and the use of a temperature of room temperature to 50° C.

In addition, the following compounds were similarly synthesized.

### 4-(1-(2-(1-methylindole-3-yl)ethyl)benzimidazole-2-ylthio)butanoic acid (Compound No. 1683)

In this case however, a methanesulfonate ester of 2-(1-methylindole-3-yl)ethanol was used instead of quaternary ammonium salt and halide derivative. Identification of the compound was carried out using LC-MS. The yield was 19% (two steps of N-alkylation and ester hydrolysis).

Calculated value M=393.15, Measured value (M+H)⁺=394.0

### 4-(1-(4-methyl-7-chlorobenzo[b]thiophene-3-yl)methyl)benzimidazole-2-ylthio)butanoic acid (Compound No. 1684)

Yield: 15% (two steps of N-alkylation and ester hydrolysis)

Calculated valve M=430.06, Measured value (M+H)⁺=431.2

¹H-NMR (270 MHz, DMSO-d₆) (ppm): 12.17 (br, 1H), 7.63 (d, 1H, J=7.83 Hz), 7.47-7.40 (m, 2H), 7.26 (d, 1H, J=8.10 Hz), 7.22-7.11 (m, 2H), 6.46 (s, 1H), 5.86 (s, 2H), 3.34 (t, 2H, J=7.29 Hz), 2.84 (s, 3H), 2.34 (t, 2H, J=7.29 Hz), 1.94 (m, 2H)

### 4-(1-(4-methyl-7-bromobenzo[b]thiphene-3-yl)methyl)benzoimidazole-2-ylthio)butanoic acid (Compound No. 1685)

Yield: 56% (two steps of N-alkylation and ester hydrolysis)

Calculated value M=474.01, Measured value (M+H)⁺=477.0

¹H-NMR (270 MHz, DMSO-d₆) (ppm): 12.18 (br, 1H), 7.63 (d, 1H, J=7.56 Hz), 7.53 (d, 1H, J=7.56 Hz), 7.46 (d, 1H, J=7.56 Hz), 7.22-7.11 (m, 3H), 6.46 (s, 1H), 5.85 (s, 2H), 3.34 (t, 2H, J=7.29 Hz), 2.83 (s, 3H), 2.34 (t, 2H, J=7.29 Hz), 1.97 (m, 2H)

| Compound | | Measured value | |
|---|---|---|---|
| No. | Calculated value M | (M + H)⁺ | Yield (two steps) % |
| 1023 | 393.15 | 394.2 | 10 |
| 1024 | 393.15 | 394.2 | 15 |
| 1025 | 393.15 | 394.1 | 25 |
| 1026 | 393.15 | 394.1 | 19 |
| 1027 | 393.15 | 394.2 | 67 |
| 1028 | 407.17 | 408.2 | 3 |
| 1029 | 413.10 | 414.3 | 74 |
| 1030 | 397.13 | 398.3 | 26 |
| 1031 | 409.15 | 410.1 | 3 |
| 1032 | 413.10 | 414.1 | 53 |
| 1033 | 397.13 | 398.1 | 56 |
| 1034 | 409.15 | 410.3 | 81 |
| 1035 | 404.13 | 405.2 | 31 |
| 1036 | 409.15 | 410.1 | 24 |
| 1039 | 416.04 | 417.3 | 100 |
| 1041 | 396.10 | 397.3 | 63 |
| 1043 | 396.10 | 397.1 | 95 |
| 1044 | 416.04 | 417.1 | 44 |
| 1048 | 410.11 | 411.3 | 33 |
| 456 | 408.17 | 408.3 | 83 |
| 1458 | 421.18 | 422.2 | 36 |
| 1460 | 441.13 | 442.3 | 58 |
| 1470 | 444.07 | 445.3 | 80 |
| 1472 | 424.13 | 425.3 | 73 |
| 1474 | 424.13 | 425.2 | 11 |
| 1544 | 461.07 | 462.0 | 89 |
| 463 | 450.00 | 451.0 | 78 |
| 1683 | 393.15 | 394.0 | 19 |
| 1684 | 430.06 | 431.2 | 15 |
| 1685 | 474.01 | 477.0 | 56 |

### Example 24

### Production of Compound No. 475

Step 1

Production of ((benzothiophene-3-yl)methyl)(4-methoxy-2-nitrophenyl)amine

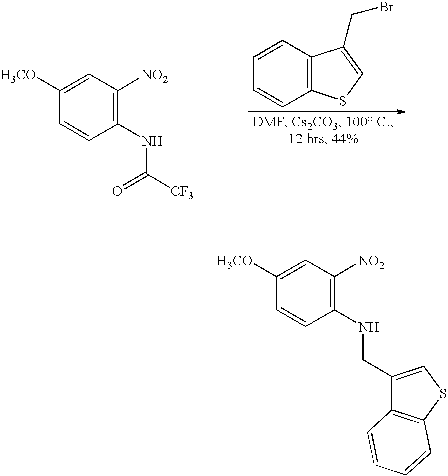

740 mg (2.8 mmol) of 4-methoxy-2-nitrotrifluoroanilide were dissolved in 5 ml of dimethylformamide followed by the sequential addition of 503 mg (3.64 mmol) of potassium carbonate and 773 mg (3.4 mmol) of 3-bromomethylbenzothiophene and heating to 100° C. After 12 hours, 5 ml of 5 M aqueous sodium hydroxide solution were added and refluxed, as is, for 1 hour. After 15 minutes, the solution was cooled to room temperature followed by the addition of 10 ml of water and extraction with chloroform. After washing the organic phase twice with 25 ml of saturated brine and drying with magnesium sulfate, it was concentrated and dried under reduced pressure. The residue was then purified by silica gel column chromatography (hexane:ethyl acetate=60:1) to obtain 400 mg of ((benzothiophene-3-yl)methyl)(4-methoxy-2-nitrophenyl)amine in the form of an orange powder (yield: 44%).

Step 2

### Production of 1-((benzothiophene-3-yl)methyl)-5-methoxybenzoimidazole-2-thiol

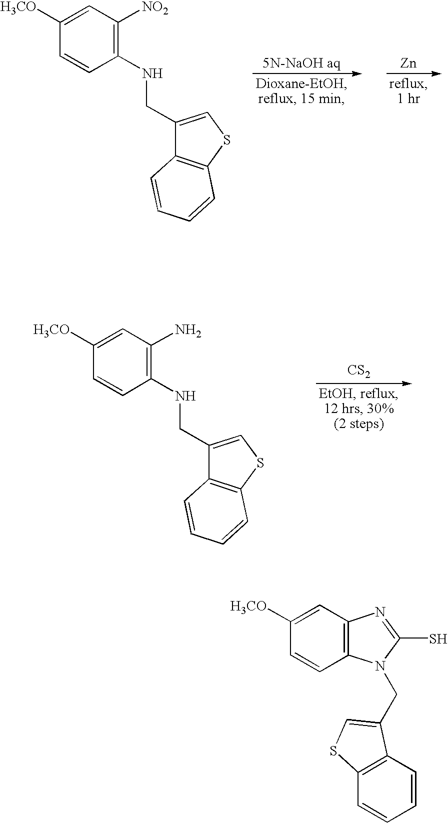

4 ml of ethanol and 4 ml of 1,4-dioxane were added to 400 mg (1.23 mmol) of ((benzothiophene-3-yl)methyl)(4-methoxy-2-nitrophenyl)amine followed by the addition of 0.34 ml of 5 M aqueous sodium hydroxide solution and refluxing while heating. After 15 minutes, the reaction solution was removed from the oil bath followed by the divided addition of 320 mg (4.9 mmol) of zinc powder. The reaction solution was again refluxed while heating for 1 hour. After allowing to cool to room temperature, the zinc was filtered out and the filtrate was concentrated under reduced pressure followed by extraction with chloroform. The organic phase was washed twice with 5 ml of saturated brine followed by drying with magnesium sulfate, concentration under reduced pressure and drying to obtain 309 mg of a brown oil.

Continuing, the resulting brown oil was dissolved in 10 ml of ethanol followed by the addition of 2.5 ml (42 mmol) of carbon disulfide and refluxing. After 12 hours, the reaction solution was returned to room temperature and concentrated under reduced pressure followed by the addition of 2 ml of ethanol and irradiating with ultrasonic waves to break into fine fragments that were then filtered. The resulting powder was washed twice with 2 ml of ethanol and then dried to obtain 120 mg (0.37 mmol) of 1-((benzothiophene-3-yl)methyl)-5-methoxybenzimidazole-2-thiol (yield of the two steps: 30%).

Step 3

### Production of 4-(1-((benzothiophene-3-yl)methyl)-5-methoxybenzimidazole-2-ylthio)butanoate ethyl ester

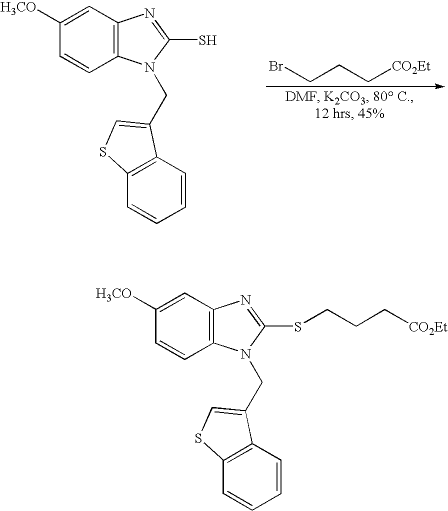

101 mg (0.30 mmol) of 1-((benzothiophene-3-yl)methyl)-5-methoxybenzimidazole-2-thiol were dissolved in 2 ml of dimethylformamide followed by the addition of 62 mg (0.45 mmol) of potassium carbonate and 53 mg (0.40 mmol) of 4-bromobutanoate ethyl ester and heating to 80° C. After 12 hours, the reaction solution was concentrated under reduced pressure and extracted with diethyl ether followed by washing twice with 10 ml of saturated brine and drying with magnesium sulfate. The solvent was then concentrated under reduced pressure and the residue was purified by silica gel column chromatography (hexane:ethyl acetate=1:1) to obtain 60 mg (0.136 mmol) of 4-(1-((benzothiophene-3-yl)methyl)-5-methoxybenzimidazole-2-ylthio)butanoate ethyl ester (yield: 45%).

Step 4

### Production of 4-(1-((benzothiophene-3-yl)methyl)-5-methoxybenzimidazole-2-ylthio)butanoic acid

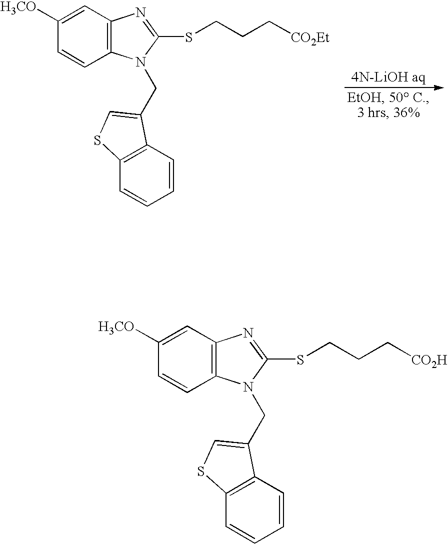

60 mg (0.136 mmol) of 4-(1-((benzothiophene-3-yl)methyl)-5-methoxybenzimidazole-2-ylthio)butanoate ethyl ester were dissolved in 2 ml of methanol followed by the addition of 0.5 ml of 4 M aqueous sodium hydroxide solution. After stirring for 3 hours at 50° C., 6 M hydrochloric acid was added to stop the reaction followed by concentrating under reduced pressure and extracting with chloroform. After washing the organic phase with saturated brine, it was dried with anhydrous magnesium sulfate. The solvent was concentrated under reduced pressure and the residue was purified by silica gel column chromatography (ethyl acetate) to obtain 20 mg (0.048 mmol) of the target compound (yield: 36%). Confirmation of the compound was carried out by identifying from the molecular weight using LC-MS.

Calculated value M=412.09, Measured value (M+H)⁺=413.1

### Example 25

### Production of Compound No. 1112

The target compound was obtained according to the same method as Example 27.

However, ((1,4-dimethylindole-3-yl)methyl) trimethylammonium iodide was used in the reaction corresponding to Step 1.

Confirmation of the compound was carried out by identifying from the molecular weight using LC-MS.

Calculated value M=423.16, Measured value (M+H)⁺=424.3

### Production of Compound No. 1114

The target compound was obtained according to the same method as Example 27.

However, ((1-methyl-4-chloroindole-3-yl)methyl) trimethylammonium iodide was used in the reaction corresponding to Step 1.

Confirmation of the compound was carried out by identifying from the molecular weight using LC-MS.

Calculated value M=443.11, Measured value (M+H)⁺=444.3

### Example 26

### Production of Compound No. 491

The target compound was obtained using the same method as Example 27. However, 4-cyano-2-nitrotrifluoroacetonitrile was used as the reagent corresponding to Step 1. In addition, the step in which the 2-nitroaniline derivative is reduced to an orthophenylenediamine derivative, and the step in which this is cyclized to a benzimidazole-2-thiol derivative were carried out using the methods described below.

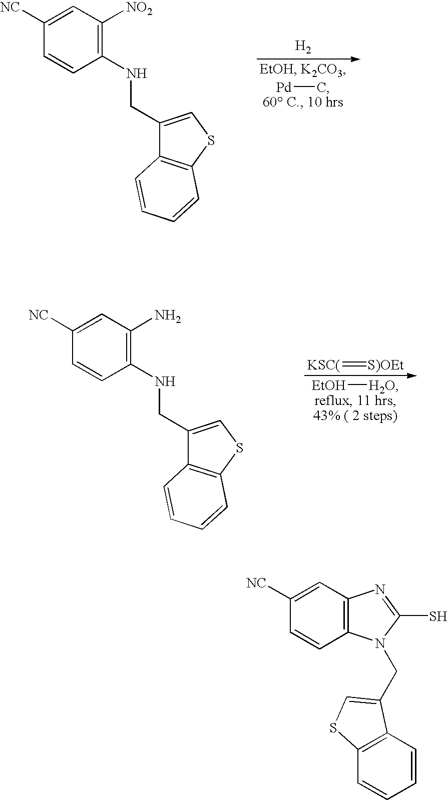

10 ml of ethanol were added to 1.1 g (3.56 mmol) of ((3-benzothiophenyl)methyl)(4-cyano-2-nitrophenyl)amine followed by the addition of 2.4 g (17.8 mmol) of potassium carbonate. After replacing the reaction system with nitrogen, 220 mg of 10% palladium-carbon were added followed by replacing the reaction system with hydrogen and heating to 60° C.

After 4 hours and 30 minutes, an additional 220 mg of 10% palladium-carbon were added followed by replacing the reaction system with hydrogen and heating to 60° C. 5 hours and 10 minutes after the start of the reaction, the reaction system was cooled to room temperature. The reaction solution was then filtered with Celite and concentrated under reduced pressure to obtain 0.93 g of a liquid residue. Continuing, 0.93 g (2.63 mmol) of ((2-benzothiophenyl)methyl)(2-amino-4-methylphenyl)amine were dissolved in 10 ml of ethanol and 2 ml of water followed by refluxing after adding 2.1 g (13.3 mmol) of potassium ethylxanthate. After 11 hours, 12.5 ml of 40% aqueous acetic acid solution were dropped in. After cooling to room temperature and concentrating under reduced pressure, the residue was purified by silica gel column chromatography (hexane:acetone=2:1) to obtain 491.7 mg of 1-((2-benzothiophenyl)methyl)-6-cyanobenzimidazole-2-thiol (yield of the two steps: 43%). Confirmation of compound no. 1209 was carried out by identifying from the molecular weight using ¹H-NMR and LC-MS.

Calculated value M=407.08, Measured value (M+H)⁺=408.2

¹H-NMR (400 MHz, CDCl₃) (ppm): 7.94 (s, 1H), 7.76 (dd, 1H), 7.52 (dd, 1H), 7.42 (m, 3H), 7.31 (d, 1H), 7.00 (s, 1H), 5.56 (s, 2H), 3.35 (t, 2H), 2.47 (t, 2H), 2.15 (p, 2H)

### Example 27

The following target compounds were obtained using the same method as Example 26.

### Production of Compound No. 471

4-methyl-2-nitrotrifluoroacetoanilide was used as the reagent corresponding to Step 1.

Confirmation of compound no. 471 was carried out by identifying from the molecular weight using LC-MS.

Calculated value M=396.10, Measured value (M+H)⁺=397.0

### Production of Compound No. 1382

5-methyl-2-nitrotrifluoroacetoanilide was used as the reagent corresponding to Step 1.

Confirmation of compound no. 1382 was carried out by identifying from the molecular weight using LC-MS.

Calculated value M=396.10, Measured value (M+H)⁺=397.0

### Example 28

### Production of Sodium Salt of Compound No. 1458

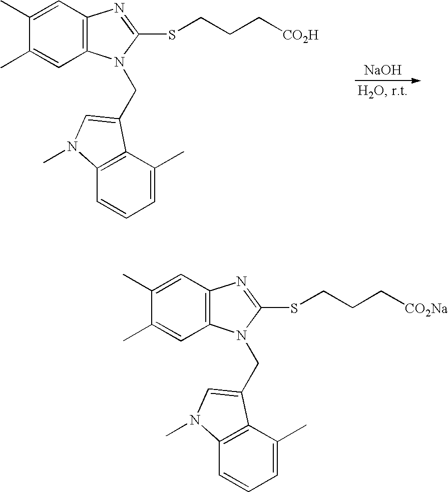

11.9 ml (1.19 mmol) of 0.1 M aqueous sodium hydroxide solution were added to 100 ml of an aqueous solution containing 503 mg (1.19 mmol) of the above compound no. 1458 followed by stirring at room temperature. Subsequently, the reaction solution was freeze-dried to obtain 470 mg (1.05 mmol) of the sodium salt (yield: 89%).

¹H-NMR (400 MHz, DMSO-d₆) (ppm): 7.37 (s, 1H), 7.19 (d, 1H, J=8.24 Hz), 7.09-7.01 (m, 2H), 6.80 (d, 1H, J=7.09 Hz), 6.32 (s, 1H), 5.66 (s, 2H), 3.59 (s, 3H), 3.26 (m, 2H), 2.66 (s, 3H), 2.27 (s, 3H), 2.21 (s, 3H), 1.95 (m, 2H), 1.81 (m, 2H)

### Example 29

The compounds indicated below were synthesized using the respective corresponding substrates according to the same method as Example 31.

### Sodium Salt of Compound No. 1027

¹H-NMR (270 MHz, DMSO-d₆) (ppm): 7.57 (d, 1H, J=Hz), 7.28 (d, 1H, J=7 Hz), 7.20 (d, 1H, J=8 Hz), 7.15-7.00 (m, 3H), 6.77 (d, 1H, J=7 Hz), 6.47 (s, 1H), 5.69 (s, 2H), 3.60 (s, 3H), 3.31 (t, 2H, J=7 Hz), 2.61 (s, 3H), 1.99 (t, 2H, J=7 Hz), 1.84 (p, 2H, J=7 Hz)

### Sodium Salt of Compound No. 459

¹H-NMR (400 MHz, DMSO-d₆) (ppm): 7.97 (d, 1H), 7.91 (d, 1H, J=6.76 Hz), 7.57 (d, 1H, J=7.75 Hz), 7.44-7.38 (m, 3H), 7.30 (s, 1H), 7.12 (m, 2H), 5.63 (s, 2H), 3.33 (m, 2H), 2.03 (m, 2H), 1.87 (m, 2H)

### Sodium Salt of Compound No. 1112

¹H-NMR (400 MHz, DMSO-d₆) (ppm): 7.21-7.00 (m, 4H), 6.79 (d, 1H, J=7.29 Hz), 6.67 (dd, 1H, J=2.43, 8.91 Hz), 6.51 (s, 1H), 5.65 (s, 2H), 3.75 (s, 3H), 3.62 (s, 3H), 3.31 (m, 2H), 2.59 (s, 3H), 1.95 (m, 2H), 1.82 (m, 2H)

### Sodium Salt of Compound No. 455

¹H-NMR (400 MHz, DMSO-d₆) (ppm): 7.98 (d, 1H, J=7.42 Hz), 7.90 (d, 1H, J=6.43 Hz), 7.44-7.39 (m, 2H), 7.35 (s, 1H), 7.18 (m, 2H), 5.57 (s, 2H), 3.28 (m, 2H), 2.26 (s, 3H), 2.23 (s, 3H), 1.99 (m, 2H), 1.84 (m, 2H)

### Example 30

### Production of 4-(1-((4-methylbenzothiophene-3-yl)methyl)-5-methoxybenzimidazole-2-ylthio)butanoate ethyl ester and 4-(1-((4-methylbenzothiophene-3-yl)methyl)-6-methoxybenzimidazole-2-ylthio)butanoateethyl ester

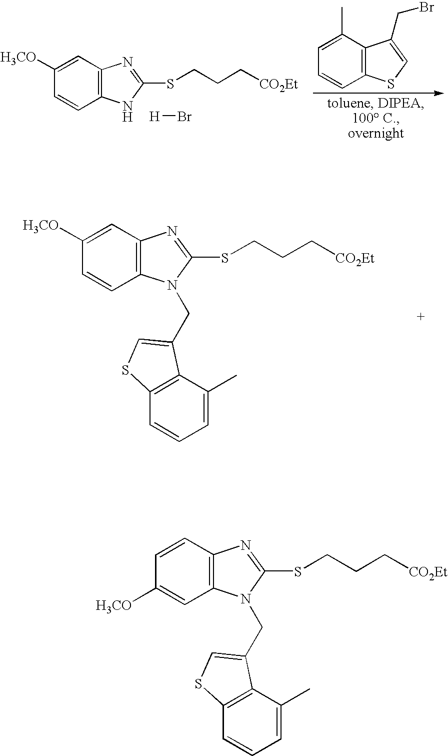

539 mg (1.44 mmol) of 4-(5-methoxybenzimidazole-2-ylthio)butanoate ethyl ester were suspended in 4 ml of toluene followed by the addition of 616 μl (3.60 mmol) of diisopropylethylamine and 384 mg (1.59 mmol) of 4-methyl-3-(bromomethyl)benzo[b]thiophene and heating at 100° C. After allowing to react overnight, saturated sodium bicarbonate solution was added followed by extraction with ethyl acetate. The organic phase was washed with water followed by drying with magnesium sulfate and concentrating the solvent under reduced pressure. The resulting residue was purified by silica gel column chromatography (hexane:ethyl acetate=4:1) to obtain 114 mg of 4-(1-((4-methylbenzothiophene-3-yl)methyl)-5-methoxybenzimidazole-2-ylthio)butanoate ethyl ester (yield: 17%) and 68 mg of 4-(1-((4-methylbenzothiophene-3-yl)methyl)-6-methoxybenzimidazole-2-ylthio)butanoate ethyl ester (yield: 10%).

### 4-(1-((4-methylbenzothiophene-3-yl)methyl)-5-methoxybenzimidazole-2-ylthio)butanoate ethyl ester

¹H-NMR (270 MHz, CDCl₃) (ppm): 7.71 (d, 1H, J=7.56 Hz), 7.62 (d, 1H, J=8.64 Hz), 7.30-7.18 (m, 2H), 6.87 (dd, 1H, J=2.43, 8.64 Hz), 6.61 (d, 1H, J=2.43 Hz), 6.42 (s, 1H), 5.74 (s, 2H), 4.10 (q, 2H, J=7.29 Hz), 3.75 (s, 3H), 3.38 (t, 2H, J=7.29 Hz), 2.89 (s, 3H), 2.45 (t, 2H, J=7.29 Hz), 2.11 (m, 2H), 1.23 (t, 3H, J=7.29 Hz)

### 4-(1-((4-methylbenzothiophene-3-yl)methyl)-6-methoxybenzimidazole-2-ylthio)butanoate ethyl ester

¹H-NMR (270 MHz, CDCl₃) (ppm): 7.70 (d, 1H, J=8.10 Hz), 7.29-7.17 (m, 3H), 7.02 (d, 1H, J=8.91 Hz), 6.80 (dd, 1H, J=2.43, 8.91 Hz), 6.40 (s, 1H), 5.74 (s, 2H), 4.11 (q, 2H, J=7.29 Hz), 3.87 (s, 3H), 3.42 (t, 2H, J=7.02 Hz), 2.88 (s, 3H), 2.46 (t, 2H, J=7.29 Hz), 2.10 (m, 2H), 1.23 (t, 3H, J=7.29 Hz)

### Example 31

The following compounds were obtained according to the same method as Example 32.

### 4-(1-((5-methylbenzothiophene-3-yl)methyl)-5-methoxybenzimidazole-2-ylthio)butanoate ethyl ester

(Yield: 24%)

¹H-NMR (270 MHz, CDCl₃) (ppm): 7.76 (d, 1H, J=8.10 Hz), 7.62 (s, 1H), 7.58 (d, 1H, J=8.64 Hz), 7.25 (1H), 6.84 (dd, 1H, J=2.43, 8.91 Hz), 6.81 (s, 1H), 6.65 (d, 1H, J=2.16 Hz), 5.47 (s, 2H), 4.11 (q, 2H, J=7.02 Hz), 3.74 (s, 3H), 3.39 (t, 2H, J=7.02 Hz), 2.51 (s, 3H), 2.47 (t, 2H, J=7.56 Hz), 2.11 (m, 2H), 1.24 (t, 3H, J=7.02 Hz)

### 4-(1-((5-methylbenzothiophene-3-yl)methyl)-6-methoxybenzimidazole-2-ylthio)butanoate ethyl ester

(Yield: 18%)

¹H-NMR (270 MHz, CDCl₃) (ppm): 7.75 (d, 1H, J=8.10 Hz), 7.60 (s, 1H), 7.26-7.22 (m, 2H), 7.04 (d, 1H, J=8.91 Hz), 6.83 (s, 1H), 6.78 (dd, 1H, J=2.43, 8.91 Hz), 5.47 (s, 2H), 4.12 (q, 2H, J=7.02 Hz), 3.84 (s, 3H), 3.43 (t, 2H, J=7.29 Hz), 2.50 (s, 3H), 2.48 (t, 2H, J=7.29 Hz), 2.12 (m, 2H), 1.24 (t, 3H, J=7.02 Hz)

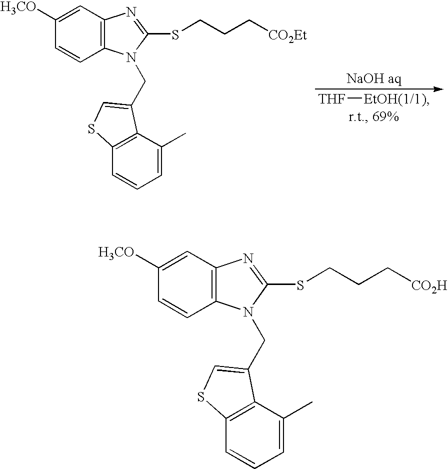

### Example 32

### Production of 4-(1-((4-methylbenzothiophene-3-yl)methyl)-5-methoxybenzimidazole-2-ylthio)butanoic acid (Compound No. 1128)

84.7 mg (0.186 mmol) of the 4-(1-((4-methylbenzothiophene-3-yl)methyl)-5-methoxybenzimidazole-2-ylthio)butanoate ethyl ester obtained in Example 32 were dissolved in a mixed solvent of 1 ml of THF and 1 ml of ethanol followed by the addition of 1 ml of 1 M aqueous sodium hydroxide solution and stirring for 1 hour at 40° C. Following completion of the reaction, 1.5 ml of 1 M hydrochloric acid were added followed by stirring for 30 minutes at room temperature. The resulting precipitate was filtered, washed with water, washed with ethanol and then dried to obtain 54.9 mg of the target compound (yield: 69%).

LC-MS:

Calculated value M=426.11, Measured value (M+H)⁺=427.2

¹H-NMR (270 MHz, DMSO-d₆) (ppm): 7.80 (d, 1H, J=7.29 Hz), 7.60 (d, 1H, J=8.91 Hz), 7.31-7.20 (m, 3H), 6.95 (dd, 1H, J=2.16, 8.91 Hz), 6.53 (s, 1H), 5.94 (s, 2H), 3.73 (s, 3H), 3.37 (t, 2H, J=7.29 Hz), 2.86 (s, 3H), 2.34 (t, 2H, J=7.29 Hz), 1.90 (m, 2H)

### Example 33

The following compounds were synthesized according to the same method as Example 32.

### 4-(1-((4-methylbenzothiophene-3-yl)methyl)-6-methoxybenzimidazole-2-ylthio)butanoic acid (Compound No. 1647)

Yield: 60%

LC-MS:

Calculated value M=426.11, Measured value (M+H)⁺=427.2

¹H-NMR (270 MHz, DMSO-d₆) (ppm): 7.78 (d, 1H, J=7.83 Hz), 7.52 (d, 1H, J=8.91 Hz), 7.34-7.17 (m, 3H), 6.77 (dd, 1H, J=2.34, 8.91 Hz), 6.37 (s, 1H), 5.83 (s, 2H), 3.78 (s, 3H), 3.32 (t, 2H, J=7.29 Hz), 2.82 (s, 3H), 2.34 (t, 2H, J=7.56 Hz), 1.93 (m, 2H)

In this case however, 1 M hydrochloric acid was added following completion of the reaction followed by extraction with chloroform and washing with water. Drying was then performed with magnesium sulfate followed by concentrating the solvent under reduced pressure and drying to obtain the target compound.

### 4-(1-((5-methylbenzothiophene-3-yl)methyl)-5-methoxybenzimidazole-2-ylthio)butanoic acid (Compound No. 1126)

Yield: 63%

LC-MS:

Calculated value M=426.11, Measured value (M+H)⁺=426.8

¹H-NMR (270 MHz, DMSO-d₆) (ppm): 7.88 (d, 1H, J=8.64 Hz), 7.76 (s, 1H), 7.58 (d, 1H, J=8.64 Hz), 7.28-7.24 (m, 3H), 6.94 (dd, 1H, J=2.16, 8.64 Hz), 5.72 (s, 2H), 3.74 (s, 3H), 3.40 (t, 2H, J=7.29 Hz), 2.42 (s, 3H), 2.36 (t, 2H, J=7.29 Hz), 1.92 (m, 2H)

### 4-(1-((5-methylbenzothiophene-3-yl)methyl)-6-methoxybenzimidazole-2-ylthio)butanoic acid (Compound No. 1645)

Yield: 79%

LC-MS:

Calculated value M=426.11, Measured value (M+H)⁺=427.0

¹H-NMR (270 MHz, DMSO-d₆) (ppm): 7.87 (d, 1H, J=8.10 Hz), 7.71 (s, 1H), 7.47 (d, 1H, J=8.91 Hz), 7.24 (m, 2H), 7.17 (d, 1H, J=2.16 Hz), 6.84 (dd, 1H), 5.64 (s, 2H), 3.77 (s, 3H), 3.38 (t, 2H, J=7.02 Hz), 2.41 (s, 3H), 2.37 (t, 2H, J=7.56 Hz), 1.95 (m, 2H)

### Example 34

### Production of HCl Salt of Compound No. 455

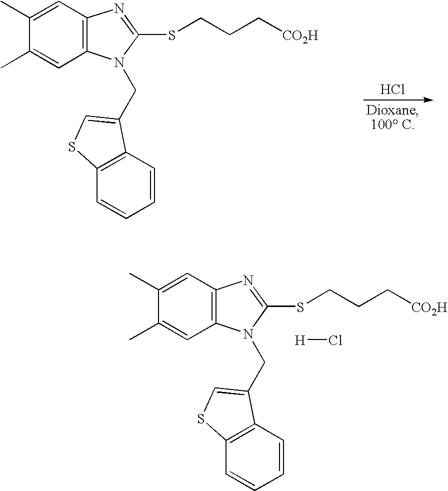

1.5 ml of 4 M hydrochloric acid/dioxane solution were added to 50 mg (0.122 mmol) of compound no. 1469 followed by stirring at 100° C. Following completion of the reaction, the reaction solution was concentrated under reduced pressure to obtain 53 mg (1.05 mmol) of the target compound (yield: 97%).

¹H-NMR (270 MHz, DMSO-d₆) (ppm): 8.00 (m, 1H), 7.89 (m, 1H), 7.52 (m, 2H), 7.45-7.42 (m, 2H), 7.32 (s, 1H), 5.78 (s, 2H), 3.48 (t, 2H, J=7.42 Hz), 2.37 (m, 2H), 2.34 (s, 3H), 2.30 (s, 3H), 1.92 (t, 2H, J=7.09 Hz)

### Example 35

### Production of HCl Salt of Compound No. 1041

The target compound was obtained according to the same method as Example 36.

¹H-NMR (270 MHz, DMSO-d₆) (ppm): 7.87 (d, 1H, J=8.08 Hz), 7.74 (s, 1H), 7.66 (d, 1H, J=6.76 Hz), 7.58 (d, 1H, J=8.74 Hz), 7.26 (m, 4H), 5.70 (s, 2H), 3.45 (t, 2H, J=7.26 Hz), 2.42 (s, 3H), 2.39 (t, 2H, J=7.26 Hz), 1.98 (m, 2H)

### Example 36

### Preparation of Recombinant Human Mast Cell Chymase

Recombinant pro-type human mast cell chymase was prepared according to the method reported by Urada et al. (Journal of Biological Chemistry 266: 17173, 1991). Thus, a culture supernatant of the insect cell (Tn5) infected with a recombinant baculovirus containing cDNA encoding human mast cell chymase was purified by heparin Sepharose (Pharmacia). After it was further activated by the method reported by Murakami et al. (Journal of Biological Chemistry 270: 2218, 1995), it was purified with heparin Sepharose to obtain an activated human mast cell chymase.

### Example 37

### Determination of the Activity of Inhibiting Recombinant Human Mast Cell Chymase

After a DMSO solution (2 μl) containing the compound of the present invention was added to 50 μl of buffer A (0.5-3.0 M NaCl, 50 mM Tris-HCl, pH 8.0) containing 1-5 ng of the activated human mast cell chymase obtained in Working Example 22, 50 μl of buffer A containing, as a substrate, 0.5 mM succinyl-alanyl-histidyl-prolyl-phenylalanylparanitroanilide (Bacchem) was added thereto and the mixture was allowed to react at room temperature for 5 minutes. Changes in absorbance at 405 nm with time were measured to evaluate the inhibitory activity.

As a result, IC50=not smaller than 1 nM and less than 10 nM was observed in compounds No. 63, 64, 65, 143, 174, 256, 264, 272, 311, 354, 319, 349, 358, 395, 401, 402, 1027, 1041, 1043, 1044, 1048, 475, 1128, 1458, 1470, 1472, 1474, 1544, 1645 and 1647, and IC50=not smaller than 10 nM and not greater than 100 nM was observed in compounds No. 37, 50, 84, 115, 117, 119, 121, 123, 130, 147, 168, 256, 320, 321, 324, 352, 355, 364, 380, 392, 398, 444, 455, 459, 460, 506, 863, 866, 869, 1026, 1029, 1030, 1039, 1112, 1114, 1126, 491, 471, 1382, 456, 1460 and 463.

As hereinabove described, the benzimidazole derivatives of the present invention exhibit a potent chymase inhibitory activity. Thus, it was revealed that the benzimidazole derivatives of the present invention are clinically applicable inhibitory substances for human chymase activity and can be used for prevention and/or therapy of various diseases in which human chymase is involved.

### Example 38

### Manufacture of Tablets

Tablets comprising, per tablet, the following were manufactured:

| | |
|---|---|
| Compound (No. 37) | 50 mg |
| Lactose | 230 mg |
| Potato starch | 80 mg |
| Polyvinylpyrrolidone | 11 mg |
| Magnesium stearate | 5 mg |

The compound of the present invention (the compound in Working Example 2), lactose and potato starch were mixed, and the mixture was evenly soaked in 20% polyvinylpyrrolidone in ethanol. The mixture was filtered through a 20 nm mesh, dried at 45° C., and filtered again through a 15 nm mesh. Granules thus obtained were mixed with magnesium stearate and were compressed into tablets.

As has been shown above, the benzimidazole derivatives of the present invention exhibit potent chymase inhibitory activity. Thus, the benzimidazole derivatives of the present invention were clearly demonstrated to be human chymase activity inhibitors that can be applied clinically for use in the prevention and/or treatment of various diseases involving human chymase.

### Example 39

### Production of Tablets

Tablets were produced having the individual tablet composition shown below.

| | |
|---|---|
| Compound No. 1027 | 50 mg |
| Lactose | 230 mg |
| Potato starch | 80 mg |
| Polyvinylpyrrolidone | 11 mg |
| Magnesium stearate | 5 mg |

The compound of the present invention (compound of the examples), lactose and potato starch were mixed followed by uniformly wetting with a 20% ethanol solution of polyvinylpyrrolidone, passing through a 20 mesh sieve, drying at 45° C. and again passing through a 15 mesh sieve. The granules obtained in this manner were then mixed with magnesium stearate and compressed into tablets.

### Example 40

### Measurement of Blood Concentration During Administration by Intragastric Forced Feeding to Rats

The compounds indicated with the above compound nos. 459, 491 and 1027 were administered by intragastric forced feeding to male SD rats while fasting at a dose of 30 mg/kg, after which blood samples were collected immediately after administration and at 30 minutes and 1, 2 and 4 hours after administration. Following collection of blood samples, where samples were immediately separated into serum components, the compound of the present invention was extracted by ordinary solid phase extraction methods, and the resulting samples were analyzed by HPLC using an ODS column (32% acetonitrile-water-0.05% TFA was used for the mobile phase for compound nos. 52 and 244, while 47% acetonitrile-water-10 mM ammonium acetate buffer (pH 4.0) was used for the mobile phase for compound no. 1027) followed by measurement of the amount of the unchanged form. Those results are shown in the table below.

| Compound No. | After 30 min. (μg/ml) | After 4 hr. (μg/ml) |
|---|---|---|
| 459 | 60.5 | 12.7 |
| 491 | 16.5 | 8.9 |
| 1027 | 16.1 | 6.3 |

On the basis of the above results, the compounds of the present invention were rapidly absorbed after administration, and blood concentrations of the unchanged form shown in the table were measured after 30 minutes. Moreover, although blood concentrations decreased gradually until 4 hours after administration, a considerable amount of the unchanged forms could still be confirmed even at 4 hours after administration. Thus, the compounds of the present invention were determined to be a group of compounds having superior pharmacokinetics properties. The pharmacokinetic properties of the group of compounds in which A is —CH₂CH₂CH₂— are particularly superior.

### Example 41

### In Vitro Metabolism Test Using Liver Microsomes (Ms)

Measurement Method:

* Reaction Solution Composition and Reaction Conditions

| | | Composition and Procedure | | |
|---|---|---|---|---|
| | Composition | Reagent name | Final conc. | Comments |
| Reconstruc-tion | Buffer | Phosphate buffer | 0.1 M | Reaction solution |
| system | | (pH 7.4) | | volume: 0.5 mL |
| Composition | Chelating agent | EDTA | 1.0 mM | |
| | NADPH | Magnesium chloride | 3.0 mM | |
| | generation | G6P | 5.0 mM | |
| | system | G6PDH | 1.0 IU | |
| | Enzyme | Liver microsomes | 1.0 mg/mL | |
| | Substrate | Substrate | 5.0 μM | |
| | | (evaluation | | |
| | | compound) | | |
| | Reaction | NADPH | 1.0 mM | |
| | initiator | | | |

| | | |
|---|---|---|
| Reaction conditions | 37° C., incubation (water bath, shaking), | |
| | reaction times: 0, 2, 5, 10 and 30 min. | |
| Reaction terminator (extraction | Acetonitrile | Equal to 3 volumes |
| liquid) | | of reaction solution |
| Deproteinization | Sampling of supernatant after | |
| | centrifuging for 10 min. at 3000 rpm, | |
| | removal of solvent with evaporator | |
| Redissolution liquid | Redissolution with HPLC mobile phase | |
| | used for analysis | |
| Analysis | Detection of peak of unchanged form by | |
| | HPLC using UV detector | |

*MR Calculation Method

The metabolic rate was determined from the decrease in the amount of the unchanged form at each reaction time and the reaction time based on assigning a value of 100% to the amount of the unchanged form at the initial concentration (reaction time: 0 minutes), and the metabolic rate at the time the metabolic rate reached a maximum was evaluated as the MR value.
MR=(substrate concentration at reaction time: 0 min.−substrate concentration after reaction)÷reaction time÷protein concentration (nmol/min./mg protein)

These methods were used to obtain the measurement results indicated below.

| | | Percentage of substrate |
|---|---|---|
| Compound No. | MR | remaining after 30 min. (%) |
| 460 | 0.260 | 60.3 |
| 1026 | 0.329 | 29.8 |
| 1027 | 0 | 80.1 |
| 1029 | 0.129 | 73.9 |
| 459 | 0.331 | 47.5 |
| 1041 | 0.111 | 41.2 |
| 1043 | 0.048 | 72.3 |
| 1112 | 0.097 | 55.2 |
| 491 | 0.211 | 57.9 |
| 456 | 0.087 | 48.7 |
| 1458 | 0.102 | 52.9 |
| 1460 | 0.088 | 61.1 |
| 455 | 0.277 | 36.2 |
| 1470 | 0.102 | 63.0 |
| 1472 | 0.131 | 56.3 |
| 1544 | 0.159 | 62.3 |

According to the above results, the compounds of the present invention are a group of metabolically stable compounds. The group of compounds in which A is —CH2CH2CH2- was determined to be a group of particularly metabolically stable

### INDUSTRIAL APPLICABILITY

The thiobenzimidazole derivatives of the present invention and the medically acceptable salts thereof exhibit a potent activity of inhibiting human chymase. Thus, said thiobenzimidazole derivatives and the medically acceptable salts thereof can be used, as a human chymase inhibitor, as clinically applicable preventive and/or therapeutic agents for inflammatory diseases, allergic diseases, diseases of respiratory organs, diseases of circulatory organs, or diseases of bone/cartilage metabolism.

1. A thiobenzimidazole compound or medically acceptable salt thereof represented by the following formula (1): 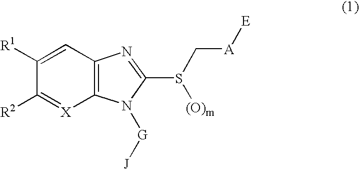
wherein,
R¹ and R² simultaneously or respectively independently represent a hydrogen atom, fluorine atom, chlorine atom, bromine atom, iodine atom, trifluoromethyl group, cyano group, hydroxyl group, methyl group, ethyl group, (n- or i-)propyl group, (n-, i-, s- or t-)butyl group, methoxy group, ethoxy group, (n- or i-)propyloxy group, (n-, i-, s- or t-)butyloxy group, or R¹ and R² together represent —O—CH₂—O—, —O—CH₂—CH₂—O— or —CH₂—CH₂—CH₂— in this case, the carbon atoms may be substituted with one or a plurality of methyl groups, ethyl groups, (n- or i-)propyl groups or (n-, i-, s- or t-)butyl groups;
A represents a substituted or non-substituted methylene group, ethylene group, (n- or i-)propylene group or (n-, i- or t-)butylene group, substituted or non-substituted phenylene group, indenylene group or naphthylene group, substituted or non-substituted pyridylene group, furanylene group, thiophenylene group, pyrimidylene group, benzophenylene group, benzimidazolene group, quinolylene group, indolene group or benzoathiazolene group and substitution groups here are represented by a halogen atom, OH, NO₂, CN, methyl group, ethyl group, (n- or i-)propyl group, (n-, i-, s- or t-)butyl group, methoxy group, ethoxy group, (n- or i-)propyloxy group, (n-, i-, s- or t-)butyloxy group, in this case, substitution groups may be acetal-bonded at mutually adjacent sites, methylthio group, ethylthio group, (n- or i-)propylthio group, (n-, i-, s- or t-)butylthio, methylsulfonyl group, ethylsulfonyl group, (n- or i-)propylsulfonyl group, (n-, i-, s- or t-)butylsulfonyl group, acetyl group, ethylcarbonyl group, (n- or i-)propylcarbonyl group, acetylamino group, ethylcarbonylamino group, (n- or i-)propylcarbonylamino group, (n-, i-, s- or t-)butylcarbonylamino group, trifluoromethyl group or trifluoromethoxy group, and one or a plurality of these may be respectively and independently substituted at an arbitrary location of a ring or alkylene group;
E represents COOR³, SO₃R³, CONHR³, SO₂NHR³, a tetrazole group, 5-oxo-1,2,4-oxadiazole group or 5-oxo-1,2,4-thiadiazole group, wherein R³ represents a hydrogen atom, methyl group, ethyl group, (n- or i-)propyl group or (n-, i-, s- or t-)butyl group;
G represents a substituted or non-substituted methylene group, ethylene group, (n- or i-)propylene group or (n-, i- or t-)butylene group, and one or a plurality of O, S, SO₂ or NR³ may be intermediately contained therein, wherein R³ is the same as previously defined, and substitution groups here are represented by a halogen atom, OH, NO₂, CN, methyl group, ethyl group, (n- or i-)propyl group, (n-, i-, s- or t-)butyl group, methoxy group, ethoxy group, (n- or i-)propyloxy group, (n-, i-, s- or t-)butyloxy group, trifluoromethyl group, trifluoromethoxy group or oxo group;
m represents an integer of 0-2;
when m is 0 and A is a substituted or non-substituted methylene group, ethylene group, (n- or i-)propylene group or (n-, i- or t-)butylene group, J represents a substituted or non-substituted (n- or i-)propyl group, (n-, i-, s- or t-)butyl group, (n-, i-, ne- or t-)pentyl group, cyclohexyl group, indenyl group, furanyl group, thiophenyl group, pyrimidyl group, benzofuranyl group, benzimidazolyl group, quinolyl group, isoquinolyl group, quinoxalyl group, benzooxadiazolyl group, benzothiadiazolyl group, indolyl group, N-methylindolyl group, benzothiazolyl group, benzothiophenyl group or benzoisooxazolyl group, substituted naphthyl group,
when m is 0 and A is a substituted or non-substituted phenylene group, indenylene group or naphthylene group, or a substituted or non-substituted pyridylene group, furanylene group, thiophenylene group, pyrimidylene group, benzophenylene group, benzimidazolene group, quinolylene group, indolene group or benzothiazolene group, J represents a substituted or non-substituted cyclohexyl group, phenyl group, indenyl group, naphthyl group, furanyl group, thiophenyl group, pyrimidyl group, benzofuranyl group, benzimidazolyl group, quinolyl group, isoquinolyl group, quinoxalyl group, benzooxadiazolyl group, benzothiadiazolyl group, indolyl group, N-methylindolyl group, benzothiazolyl group, benzothiophenyl group or benzoisooxazolyl group;
when m is 0 and A is a single bond or when m is 1 or 2, J represents a substituted or non-substituted cyclohexyl group, phenyl group, indenyl group, naphthyl group, furanyl group, thiophenyl group, pyrimidyl group, benzofuranyl group, benzimidazolyl group, quinolyl group, isoquinolyl group, quinoxalyl group, benzooxadiazolyl group, benzothiadiazolyl group, indolyl group, N-methylindolyl group, benzothiazolyl group, benzothiophenyl group or benzoisooxazolyl group; substitution groups here are represented by a halogen atom, OH, NO₂, CN, methyl group, ethyl group, (n- or i-)propyl group, (n-, i-, s- or t-)butyl group, methoxy group, ethoxy group, (n- or i-)propyloxy group, (n-, i-, s- or t-)butyloxy group, methylthio group, ethylthio group, (n- or i-)propylthio group, (n-, i-, s- or t-)butylthio group, methylsulfonyl group, ethylsulfonyl group, (n- or i-)propylsulfonyl group, (n-, i-, s- or t-)butylsulfonyl group, acetyl group, ethylcarbonyl group, (n- or i-)propylcarbonyl group, acetylamino group, ethylcarbonylamino group, (n- or i-)propylcarbonylamino group, (n-, i-, s- or t-)butylcarbonylainino group, trifluoromethyl group or trifluoromethoxy group, and one or a plurality of these may be respectively and independently substituted at an arbitrary location of a ring or alkyl group; and,
X represents CH or a nitrogen atom.

2. A thiobenzimidazole compound or medically acceptable salt thereof represented by the following formula (1), wherein,
R¹ and R² simultaneously or respectively independently represent a hydrogen atom, fluorine atom, chlorine atom, bromine atom, iodine atom, trifluoromethyl group, cyano group, hydroxyl group, methyl group, ethyl group, (n- or i-)propyl group, (n-, i-, s- or t-)butyl group, methoxy group, ethoxy group, (n- or i-)propyloxy group, (n-, i-, s- or t-)butyloxy group, or R¹ and R² together represent —O—CH₂—O—, —O—CH₂—CH₂—O— or —CH₂—CH₂—CH₂— in this case, the carbon atoms may be substituted with one or a plurality of methyl groups, ethyl groups, (n- or i-)propyl groups or (n-, i-, s- or t-)butyl groups;
A represents a substituted or non-substituted methylene group, ethylene group, (n- or i-)propylene group or (n-, i- or t-)butylene group, and substitution groups here are represented by a fluorine atom, chlorine atom, bromine atom, iodine atom, OH, NO₂, CN, methyl group, ethyl group, (n- or i-)propyl group, (n-, i-, s- or t-)butyl group, methoxy group, ethoxy group, (n- or i-)propyloxy group, (n-, i-, s- or t-)butyloxy group, in this case, substitution groups may be acetal-bonded at mutually adjacent sites, methylthio group, ethylthio group, (n- or i-)propylthio group, (n-, i-, s- or t-)butylthio group, methylsulfonyl group, ethylsulfonyl group, (n- or i-)propylsulfonyl group, (n-, i-, s- or t-)butylsulfonyl group, acetyl group, ethylcarbonyl group, (n- or i-)propylcarbonyl group, acetylamino group, ethylcarbonylamino group, (n- or i-) propylcarbonylamino group, (n-, i-, s- or t-)butylcarbonylamino group, trifluoromethyl group or trifluoromethoxy group, and one or a plurality of these may be respectively and independently substituted at an arbitrary location of alkylene group;
E represents COOR³, SO₃R³, CONHR³, SO₂NHR³, tetrazole-5-yl group, 5-oxo-1,2,4-oxadiazole-3-yl group or 5-oxo-1,2,4-thiadiazole-3-yl group wherein, R³ represents a hydrogen atom, methyl group, ethyl group, (n- or i-)propyl group or (n-, i-, s- or t-)butyl group;
G represents a substituted or non-substituted methylene group, ethylene group, (n- or i-)propylene group or (n-, i- or t-)butylene group, and one or a plurality of O, S, SO₂ or NR³ may be intermediately contained therein, wherein R³ is the same as previously defined, and substitution groups here are represented by a fluorine atom, chlorine atom, bromine atom, iodine atom, OH, NO₂, CN, methyl group, ethyl group, (n- or i-)propyl group, (n-, i-, s- or t-) butyl group, methoxy group, ethoxy group, (n- or i-)propyloxy group, (n-, i-, s- or t-)butyloxy group, trifluoromethyl group, trifluoromethoxy group or oxo group;
m represents an integer of 0-2;
J represents a substituted or non-substituted furanyl group, thiophenyl group, pyrimidyl group, benzofuranyl group, benzimidazolyl group, quinolyl group, isoquinolyl group, quinoxalyl group, benzooxadiazolyl group, benzothiadiazolyl group, indolyl group, benzothiazolyl group, benzothiophenyl group or benzoisooxazolyl group; substitution groups here are represented by a fluorine group, chlorine group, bromine group, iodine group, OH, NO₂, CN, methyl group, ethyl group, (n- or i-)propyl group, (n-, i-, s- or t-)butyl group, methoxy group, ethoxy group, (n- or i-)propyloxy group, (n-, i-, s- or t-)butyloxy group, methylthio group, ethylthio group, (n- or i-)propylthio group, (n-, i-, s- or t-)butylthio group, methylsulfonyl group, ethylsulfonyl group, (n- or i-)propylsulfonyl group, (n-, i-, s- or t-)butylsulfonyl group, acetyl group, ethylcarbonyl group, (n- or i-)propylcarbonyl group, acetylamino group, ethylcarbonylamino group, (n- or i-)propylcarbonylamino group, (n-, i-, s- or t-)butylcarbonylamino group, trifluoromethyl group or trifluoromethoxy group, and one or a plurality of these may be respectively and independently substituted at an arbitrary location of a ring; and,
X represents CH or a nitrogen atom.

3. The thiobenzimidazole compound or medically acceptable salt thereof according to claim 1, wherein, in the above formula (1), A is a substituted or non-substituted methylene group, ethylene group, (n- or i-)propylene group or (n-, i- or t-)butylene group, a substituted or non-substituted phenylene group, indenylene group, naphthylene group, or a substituted or non-substituted pyridylene group, furanylene group, thiophenylene group, pyrimidylene group, benzophenylene group, benzimidazolene group, quinolylene group, indolene group or benzothiazolene group.

4. The thiobenzimidazole compound or medically acceptable salt thereof according to claim 1, wherein in the above formula (1), A is a substituted or non-substituted pyridylene group, furanylene group, thiophenylene group, pyrimidylene group, benzophenylene group, benzimidazolene group, quinolylene group, indolene group or benzothiazolene group.

5. The thiobenzimidazole compound or medically acceptable salt thereof according to claim 1, wherein in the formula (1), A is a substituted or non-substituted ethylene group.

6. The thiobenzimidazole compound or medically acceptable salt thereof according to claim 1, wherein, in the above formula (1), m is 1.

7. The thiobenzimidazole compound or medically acceptable salt thereof according to claim 1, wherein, in the above formula (1), m is 2.

8. The thiobenzimidazole compound or medically acceptable salt thereof according to claim 1, wherein, in the above formula (1), m is 0, A is a substituted or non-substituted methylene group, ethylene group, (n- or i-)propylene group or (n-, i- or t-)butylene group, and J is a substituted or non-substituted indenyl group or substituted naphthyl group.

9. The thiobenzimidazole compound or medically acceptable salt thereof according to claim 1, wherein, in the above formula (1), m is 0, A is a substituted or non-substituted methylene group, ethylene group, (n- or i-)propylene group or (n-, i- or t-)butylene group, and J is a substituted or non-substituted furanyl group, thiophenyl group, pyrimidyl group, benzofuranyl group, benzimidazolyl group, quinolyl group, isoquinolyl group, quinoxalyl group, benzooxadiazolyl group, benzothiadiazolyl group, indolyl group, N-methylindolyl group, benzothiazolyl group, benzothiophenyl group or benzoisooxazolyl group.

10. The thiobenzimidazole compound or medically acceptable salt thereof according to claim 1, wherein, in the above formula (1), m is 0, A is a substituted or non-substituted phenylene group, indenylene group or naphthylene group, a substituted or non-substituted pyridylene group, furanylene group, thiophenylene group, pyrimidylene group, benzophenylene group, benzimidazolene group, quinolylene group, indolene group or benzothiazolene group, and J is a substituted or non-substituted phenyl group, indenyl group or naphthyl group, or a substituted or non-substituted furanyl group, thiophenyl group, pyrimidyl group, benzofuranyl group, benzimidazolyl group, quinolyl group, isoquinolyl group, quinoxalyl group, benzooxadiazolyl group, benzothiadiazolyl group, indolyl group, N-methylindolyl group, benzothiazolyl group, benzothiophenyl group or benzoisooxazolyl group.

11. The thiobenzimidazole compound or medically acceptable salt thereof according to claim 1, wherein in the above formula (1), J is a substituted or unsubstituted indolyl group or benzothiophenyl group.

12. The thiobenzimidazole compound or medically acceptable salt thereof according to claim 1, wherein, in the above formula (1), G is —CH₂—, —CH₂CH₂—, —CH₂CO—, —CH₂CH₂O—, —CH₂CONH—, —CO—, —CH₂SO₂—, —CH₂S— or —CH₂CH₂S—.

13. The thiobenzimidazole compound or medically acceptable salt thereof according to claim 1, wherein, in the above formula (1), R¹ and R² are simultaneously a hydrogen atom, halogen atom, methyl group, ethyl group, (n- or i-)propyl group, (n-, i-, s- or t-)butyl group, methoxy group, ethoxy group, (n- or i-)propyloxy group or (n-, i-, s- or t-)butyloxy group, or R¹ and R² are respectively and independently a hydrogen atom, halogen atom, methyl group, ethyl group, (n- or i-)propyl group, (n-, i-, s- or t-)butyl group, methoxy group, ethoxy group, (n- or i-)propyloxy group, (n-, i-, s-, or t-)butyloxy group, triflluoromethyl group, cyano group or hydroxyl group.

14. The thiobenzimidazole compound or medically acceptable salt thereof according to claim 1, wherein in the above formula (1), R¹ and R² simultaneously or respectively independently represent a hydrogen atom, fluorine atom, chlorine atom, methyl group, ethyl group, (n- or i-)propyl group, (n-, i-, s- or t-)butyl group, methoxy group, ethoxy group, (n- or i-)propyloxy group, (n-, i-, s- or t-)butyloxy group, trifluoromethyl group, cyano group, or hydroxy group.

15. The thiobenzimidazole compound or medically acceptable salt thereof according to claim 1, wherein, in the above formula (1), E is COOH or a tetrazole group.

16. The thiobenzimidazole compound or medically acceptable salt thereof according to claim 1, wherein, in the above formula (1), X is CH.

17. A pharmaceutical composition comprising at least one thiobenzimidazole compound or medically acceptable salt thereof according to claim 1, and a pharmaceutically acceptable carrier.

18. A method for inhibiting human chymase by administering to a human subject an effective amount of a pharmaceutical composition comprising a thiobenzimidazole compound according to claim 1 as the active ingredient and a pharmaceutically acceptable carrier.

19. A method for inhibiting human chymase by administering to a human subject an effective amount of a pharmaceutical composition comprising a thiobenzimidazole compound according to claim 9 as the active ingredient and a pharmaceutically acceptable carrier.

20. A method for treating an allergic disease, bronchial asthma, cardiovascular disease selected from the group consisting of sclerosing vascular lesions, peripheral circulation disorders, renal insufficiency and cardiac insufficiency, and bone/cartilage metabolic diseases selected from the group consisting of rheumatoid arthritis and osteoarthritis by administering to a human subject an effective amount of a pharmaceutical composition comprising a thiobenzimidazole compound according to claim 1 as the active ingredient.

21. A method for treating an allergic disease, bronchial asthma, cardiovascular disease selected from the group consisting of sclerosing vascular lesions, peripheral circulation disorders, renal insufficiency and cardiac insufficiency, and bone/cartilage metabolic diseases selected from the group consisting of rheumatoid arthritis and osteoarthritis by administering to a human subject an effective amount of a pharmaceutical composition comprising a thiobenzimidazole compound according to claim 9 as the active ingredient.
